(19) United States
(12) Patent Application Publication
(10) Pub. No.: US 2025/0049809 A1
(43) Pub. Date: Feb. 13, 2025

(54) INHIBITORS OF MUTANT IDH1 AND IDH2

(71) Applicant: Board of Regents, The University of Texas System, Austin, TX (US)

(72) Inventors: Fernando G. ALVAREZ, Houston, TX (US); Pijus K. MANDAL, Sugar Land, TX (US); Matthew C. HORTON, Missouri City, TX (US); Michael J. SOTH, Sugar Land, TX (US); Kang LE, Sugar Land, TX (US); Philip JONES, Houston, TX (US); Timothy J. MCAFOOS, Pearland, TX (US); Priyanka SRIVASTAVA, League City, TX (US)

(21) Appl. No.: 18/787,216

(22) Filed: Jul. 29, 2024

Related U.S. Application Data
(60) Provisional application No. 63/516,613, filed on Jul. 31, 2023.

(51) Int. Cl. A61K 31/5383; A61K 45/06; A61P 35/02; C07D 498/16

(52) U.S. Cl. CPC A61K 31/5383; A61K 45/06; A61P 35/02; C07D 498/16

(57) ABSTRACT

Disclosed herein are compounds, and salts thereof, which inhibit mutant IDH proteins. Also disclosed are pharmaceutical formulations and methods of treatment for diseases associated with an IDH protein having one or more neomorphic mutations, such as certain cancers.

This application claims the benefit of priority of U.S. Provisional Application No. 63/516,613, filed Jul. 31, 2023, the contents of which are incorporated by reference as if written herein in their entirety.

Isocitrate dehydrogenase (IDH) proteins catalyze the oxidative decarboxylation of isocitrate to alpha-ketoglutarate while also reducing NADP+ to NADPH. Codon mutations on IDH genes confer neomorphic activity, reducing the affinity for isocitrate and resulting in higher activity for the conversion of alpha-ketoglutarate to 2-hydroxyglutarate (2-HG). 2-HG has been shown to be an inhibitor of dioxygenases, which help regulate DNA demethylase and histone demethylase. While low levels of 2-HG are naturally produced with wild-type IDH and can be cleared rather quickly, mutant IDH1 and mutant IDH2 produce high levels of accumulated 2-HG that can inhibit dioxygenase activity. This leads to aberrant epigenetic hypermethylation and has been shown to drive oncogenesis in several cancers. Notably, inhibitors of mutant IDH have been clinically validated and some have been approved for AML. Inhibitors of mutant IDH are expected to be useful as drugs to treat a variety of cancers.

Despite the attention focused on inhibitors of mutant IDH, there exists a need for compounds and methods that are optimized for activity in resistance IDH mutations, including, but not limited to IDH1R132H/S280F and IDH2R140Q, that come about from clinical resistance of first-in-line mutant IDH therapies. These offer a superior profile with respect to pharmacokinetic properties, potency, or off-target activities, which result in an improved efficacy/tolerability profile and better patient outcomes.

The present disclosure fulfills these and other needs, as evident in reference to the following disclosure.

### SUMMARY

Provided is a compound of Formula I or Formula II,

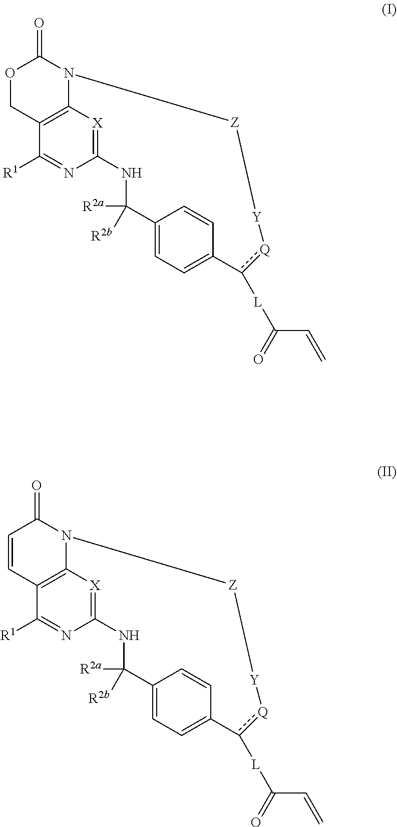

or a pharmaceutically acceptable salt thereof, wherein
   L is chosen from C₁₋₆ alkylene, C₃₋₇ cycloalkylene, 3- to 7-membered heterocycloalkylene, arylene, and heteroarylene, any of which may be optionally substituted with one or more R³;
   Q is chosen from —O—, —CR^{c}R^{d}—, and ═CR^{c}—;
   X is chosen from CH, CF, and N;
   Y is absent or is chosen from arylene and heteroarylene, either of which may be optionally substituted with one or more R³;
   Z is a saturated or partially unsaturated bivalent C₄₋₁₀ hydrocarbon chain optionally substituted with one or more R^{a}, wherein 1 or 2 nonconsecutive methylene units of the chain may be independently replaced by —O—, —OC(O)—, —C(O)O—, —C(O)—, —NR^{b}C(O)—, —C(O)NR^{b}—, —OC(O)NR^{b}—, or —NR^{b}C(O)O—;
   each R^{a} is independently chosen from C₁₋₆ alkyl, C₃₋₈ cycloalkyl, halo, C₁₋₆ haloalkyl, C₁₋₆ alkoxy, aryl, and heteroaryl;
   each R^{b} is independently chosen from hydrogen and C₁₋₆ alkyl;
   R^{c} is chosen from hydrogen, C₁₋₆ alkyl, C₃₋₈ cycloalkyl, halo, C₁₋₆ haloalkyl, C₁₋₆ alkoxy, aryl, and heteroaryl;
   R^{d} is chosen from hydrogen and C₁₋₆ alkyl;
   R¹ is chosen from hydrogen, halo, hydroxyl, and amino;
   R^{2a} and R^{2b} are independently chosen from hydrogen, methyl, and halomethyl; and
   each R³ is independently chosen from C₁₋₆ alkyl, C₃₋₇ cycloalkyl, C₁₋₆haloalkyl, 3- to 7-membered heterocycloalkyl, C₁₋₆ alkoxy, C₁₋₆ haloalkoxy, cyano, halo, and hydroxy.

Also provided is a compound as disclosed herein, or a pharmaceutically acceptable salt thereof.

Also provided is a pharmaceutical formulation comprising a compound as disclosed herein, or a pharmaceutically acceptable salt thereof, together with a pharmaceutically acceptable carrier.

Also provided is a method for treating a disease or condition that benefits from or is treatable by inhibition of mutant IDH, comprising the administration of a therapeutically effective amount of a compound described herein, or a pharmaceutically acceptable salt thereof, to a subject in need thereof.

Also provided are methods of inhibiting at least one mutant IDH function comprising the step of contacting mutant IDH with a compound as described herein, or a pharmaceutically acceptable salt thereof. The cell phenotype, cell proliferation, activity of mutant IDH, change in biochemical output produced by active mutant IDH, expression of mutant IDH, or binding of mutant IDH with a natural binding partner may be monitored. Such methods may be modes of treatment of disease, biological assays, cellular assays, biochemical assays, or the like.

Also provided are methods of treatment of an mutant IDH-mediated disease comprising the administration of a therapeutically effective amount of a compound as disclosed herein, or a pharmaceutically acceptable salt thereof, to a patient in need thereof.

Also provided is a method of inhibition of mutant IDH comprising contacting mutant IDH with a compound as disclosed herein, or a pharmaceutically acceptable salt thereof.

Also provided is a method of modulation of an mutant IDH-mediated function in a subject comprising the administration of a therapeutically effective amount of a compound as disclosed herein, or a pharmaceutically acceptable salt thereof.

These and other aspects of the invention will be apparent upon reference to the following detailed description.

### DETAILED DESCRIPTION

As used in the present specification, the following words and phrases are generally intended to have the meanings as set forth below, except to the extent that the context in which they are used indicates otherwise.

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” or “some embodiments” or “a certain embodiment” means that a particular feature, structure or characteristic described in connection with the embodiment is included in at least one embodiment. Thus, the appearances of the phrases “in one embodiment” or “in an embodiment” or “in some embodiments” or “in a certain embodiment” in various places throughout this specification are not necessarily all referring to the same embodiment. Furthermore, the particular features, structures, or characteristics may be combined in any suitable manner in one or more embodiments.

Also, as used in this specification and the appended claims, the singular forms “a,” “an,” and “the” include plural referents unless the content clearly dictates otherwise.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “alkoxy”, and, interchangeably, “(alkyl)oxy”, as used herein, refers to an alkyl radical attached to a molecule by oxygen.

The term “alkyl,” as used herein, refers to a straight-chain or branched-chain saturated, hydrocarbon radical containing from 1 to 20 carbon atoms. In some embodiments, alkyl will comprise from 1 to 10 carbon atoms. In some embodiments, alkyl will comprise from 1 to 8 carbon atoms. The term “alkylene” refers to a bivalent alkyl group.

The term “amino,” as used herein, refers to —NRR′, wherein R and R′ are independently chosen from hydrogen, alkyl, acyl, heteroalkyl, aryl, cycloalkyl, heteroaryl, and heterocycloalkyl. Additionally, R and R′ may combine with the amino nitrogen atom to form heterocycloalkyl. In some embodiments, R and R′ are both hydrogen. In some embodiments, amino is —NH₂.

The term “aryl,” as used herein, means a carbocyclic aromatic system containing one, two or three rings wherein such polycyclic ring systems are fused together. The term “arylene” refers to a bivalent aryl group.

The term “cycloalkoxy,” as used herein, refers to a cycloalkyl group attached to the parent molecular moiety through an oxygen atom.

The term “cycloalkyl,” or, alternatively, “carbocycle,” as used herein, refers to a saturated monocyclic, bicyclic or tricyclic alkyl group, wherein each cyclic moiety contains from 3 to 12 carbon atom ring members. In some embodiments, cycloalkyl will comprise from 5 to 7 carbon atoms. In some embodiments, cycloalkyl will comprise a spirocyclic ring system. “Bicyclic” and “tricyclic” as used herein are intended to include both fused ring systems, as well as the multicyclic (multicentered) saturated type. The term “cycloalkylene” refers to a bivalent cycloalkyl group.

The term “halo,” or “halogen,” as used herein, refers to fluorine, chlorine, bromine, or iodine.

The term “haloalkyl,” as used herein, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals.

The term “haloalkoxy,” as used herein, refers to a haloalkyl group attached to the parent molecular moiety through an oxygen atom.

The term “halomethyl,” as used herein, refers to a methyl group substituted by one, two, or three halogen groups. In some embodiments, halomethyl is trifluoromethyl. In some embodiments, halomethyl is monofluoromethyl.

The term “heteroaryl,” as used herein, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In some embodiments, heteroaryl will comprise from 1 to 4 heteroatoms as ring members. In some embodiments, heteroaryl will comprise from 1 to 2 heteroatoms as ring members. In some embodiments, heteroaryl will comprise from 5 to 7 atoms. The term also embraces fused polycyclic groups wherein heterocyclic rings are fused with aryl rings wherein heteroaryl rings are fused with other heteroaryl rings wherein heteroaryl rings are fused with heterocycloalkyl rings, or wherein heteroaryl rings are fused with cycloalkyl rings. The term “heteroarylene” refers to a bivalent heteroaryl group.

The term “heterocycloalkoxy,” as used herein, refers to a heterocycloalkyl group attached to the parent molecular moiety through an oxygen atom.

The terms “heterocycloalkyl” and, interchangeably, “heterocycle,” as used herein, refers to a saturated, partially unsaturated, or fully unsaturated (but nonaromatic) monocyclic; saturated, partially unsaturated, or fully unsaturated (but not fully aromatic) bridged; saturated, partially unsaturated, or fully unsaturated (but not fully aromatic) bicyclic; or saturated, partially unsaturated, or fully unsaturated (but not fully aromatic) tricyclic heterocyclic group containing at least one heteroatom as a ring member wherein each heteroatom may be independently chosen from nitrogen, oxygen, and sulfur.

In some embodiments, heterocycloalkyl will comprise a spirocyclic ring system. In some embodiments, heterocycloalkyl will comprise from 1 to 4 heteroatoms as ring members. In some embodiments, heterocycloalkyl will comprise from 1 to 2 heteroatoms as ring members. In some embodiments, heterocycloalkyl will comprise from 3 to 8 ring members in each ring. In some embodiments, heterocycloalkyl will comprise from 3 to 7 ring members in each ring. In some embodiments, heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl or heteroaryl group, as defined herein, or an additional heterocycle group. The term “heterocycloalkylene” refers to a bivalent heterocycloalkyl group.

Asymmetric centers exist in the compounds and pharmaceutically acceptable salts thereof, disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds, and pharmaceutically acceptable salts thereof, can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds, and pharmaceutically acceptable salts thereof, of particular stereochemistry are either commercially available or can be made and resolved by techniques known in the art. Additionally, the compounds, and pharmaceutically acceptable salts thereof, disclosed herein may exist as geometric isomers. The present disclosure includes all cis, trans, syn, anti, entgegen (E), and zusammen (Z) isomers as well as the appropriate mixtures thereof.

Additionally, the compounds disclosed herein can exist in unsolvated as well as solvated forms with pharmaceutically acceptable solvents such as water, ethanol, and the like. In general, the solvated forms are considered equivalent to the unsolvated forms.

As used herein, “administering to a patient” refers to the process of introducing a composition or dosage form into the patient via an art-recognized means of introduction.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treatment of a disease or disorder or on the effecting of a clinical endpoint. The precise therapeutically effective amount for a subject may depend upon, e.g., the subject's size and health, the nature and extent of the condition, the therapeutics or combination of therapeutics selected for administration, and other variables known to those of skill in the art. The effective amount for a given situation is determined by routine experimentation and is within the judgment of the clinician.

As used herein, the term “treat,” “treating”, or “treatment” means the administration of therapy to an individual who already manifests at least one symptom of a disease or condition or who has previously manifested at least one symptom of a disease or condition. For example, “treating” can include alleviating, abating or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

Those skilled in the art will appreciate that the invention(s) described herein is susceptible to variations and modifications other than those specifically described. It is to be understood that the invention(s) includes all such variations and modifications. The invention(s) also includes all the steps, features, compositions and compounds referred to or indicated in this specification, individually or collectively, and any and all combinations or any two or more of steps or features unless specifically stated otherwise.

The present invention(s) is not to be limited in scope by the specific embodiments described herein, which are intended for the purpose of exemplification only. Functionally equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination.

Provided is a compound of Formula I or Formula II,

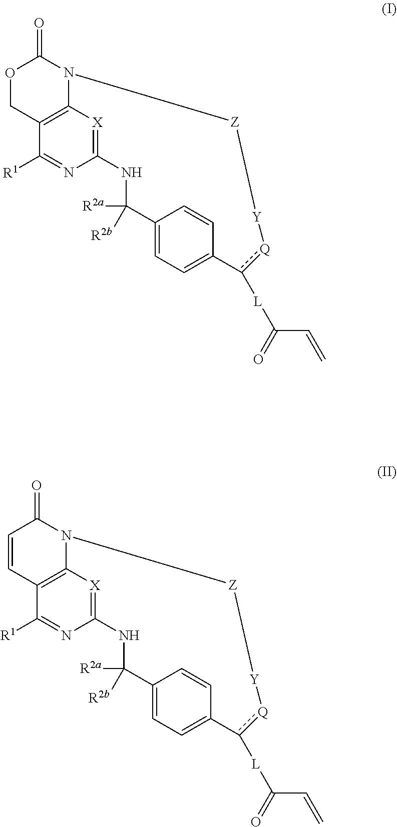

or a pharmaceutically acceptable salt thereof, wherein
   L is chosen from C₁₋₆ alkylene, C₃₋₇ cycloalkylene, 3- to 7-membered heterocycloalkylene, arylene, and heteroarylene, any of which may be optionally substituted with one or more R³;
   Q is chosen from —O—, —CR^{c}R^{d}—, and ═CR^{c}—;
   X is chosen from CH, CF and N;
   Y is absent or is chosen from arylene and heteroarylene, either of which may be optionally substituted with one or more R³;
   Z is a saturated or partially unsaturated bivalent C₄₋₁₀ hydrocarbon chain optionally substituted with one or more R^{a}, wherein 1 or 2 nonconsecutive methylene units of the chain may be independently replaced by —O—, —OC(O)—, —C(O)O—, —C(O)—, —NR^{b}C(O)—, —C(O)NR^{b}—, —OC(O)NR^{b}—, or —NR^{b}C(O)O—;
   each R^{a} is independently chosen from C₁₋₆ alkyl, C₃₋₈ cycloalkyl, halo, C₁₋₆ haloalkyl, C₁₋₆ alkoxy, aryl, and heteroaryl;
   each R^{b} is independently chosen from hydrogen and C₁₋₆ alkyl;
   R^{c} is chosen from hydrogen, C₁₋₆ alkyl, C₃₋₈ cycloalkyl, halo, C₁₋₆ haloalkyl, C₁₋₆ alkoxy, aryl, and heteroaryl;
   R^{d} is chosen from hydrogen and C₁₋₆ alkyl;
   R¹ is chosen from hydrogen, halo, hydroxyl, and amino;
   R^{2a} and R^{2b} are independently chosen from hydrogen, methyl, and halomethyl; and
   each R³ is independently chosen from C₁₋₆ alkyl, C₃₋₇ cycloalkyl, C₁₋₆ haloalkyl, 3- to 7-membered heterocycloalkyl, C₁₋₆ alkoxy, C₁₋₆ haloalkoxy, cyano, halo, and hydroxy.

In some embodiments, L is 3- to 7-membered heterocycloalkylene.

In some embodiments, L is chosen from piperazinyl and piperidinyl.

In some embodiments, L is

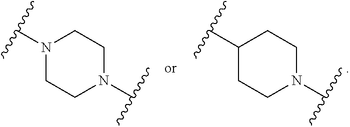 [SMILES: *N1CCN(*)CC1 ; *C1CCN(*)CC1]

In some embodiments, wherein X is N.

In some embodiments, wherein X is CH.

In some embodiments, R¹ is hydrogen.

In some embodiments, R^{2a} and R^{2b} are independently chosen from hydrogen and methyl.

In some embodiments, R^{2a} is methyl and R^{2b} is hydrogen.

In some embodiments, a compound as described herein has the structure of Formula IA or IIA

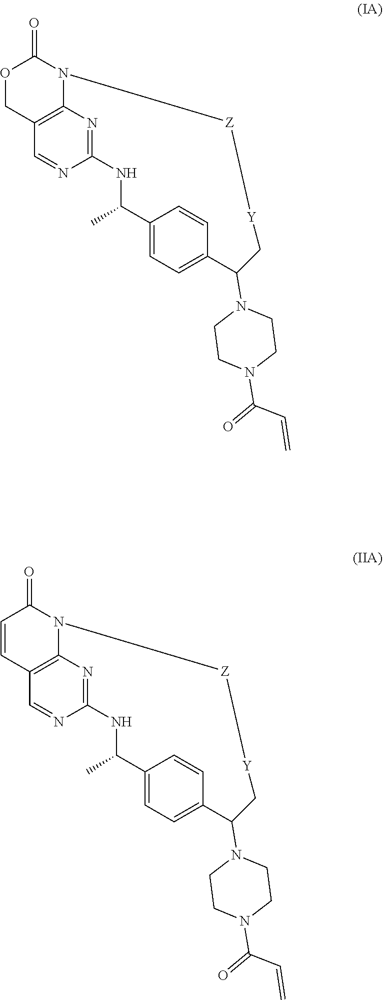

or a pharmaceutically acceptable salt thereof.

In some embodiments, Z is —(CH₂)₈—.

In some embodiments, Z is —(CH₂)₇—.

In some embodiments, Z is —(CH₂)₄—CH═CH—(CH₂)₂—.

In some embodiments, Z is —(CH₂)₄—CH═CH—CH₂—.

In some embodiments, Z is —(CH₂)₃—CH═CH—CH₂—.

In some embodiments, a compound as described herein has the structure of Formula IB or IIB

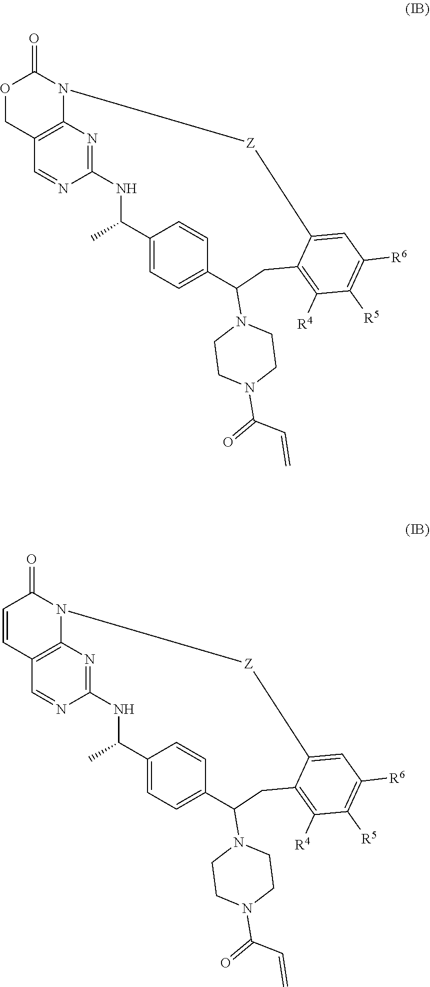

or a pharmaceutically acceptable salt thereof, wherein:
   R⁴ is chosen from hydrogen, C₁₋₆ alkyl, C₁₋₆ haloalkyl, C₁₋₆ haloalkoxy, C₃₋₇ cycloalkyl, and halo;
   R⁵ is chosen from hydrogen, C₁₋₆ alkyl, C₁₋₆ haloalkyl, C₁₋₆ haloalkoxy, and C₃₋₇ cycloalkyl; and
   R⁶ is chosen from hydrogen, C₁₋₆ alkyl, and C₁₋₆ haloalkyl.

In some embodiments, R⁴ is chosen from hydrogen, methyl, trifluoromethyl, trifluoromethoxy, cyclopropyl, and fluoro.

In some embodiments, R⁵ is chosen from hydrogen, methyl, trifluoromethyl, trifluoromethoxy, and cyclopropyl.

In some embodiments, R⁶ is chosen from hydrogen, methyl, and trifluoromethyl.

In some embodiments, Z is chosen from —O(CH₂)₄— and —(CH₂)₅—, either of which are optionally substituted with one R^{a}.

In some embodiments, Z is —O(CH₂)₄—.

In some embodiments, Z is —(CH₂)₅—.

In some embodiments, Q is chosen from —O— and —CR^{c}R^{d}—.

In some embodiments, R^{c} and R^{d} are hydrogen.

In some embodiments, R^{c} is cyclopropyl and R^{d} is hydrogen.

In some embodiments, Y is absent.

In some embodiments, Z is —(CH₂)₅— or —(CH₂)₆—, either of which are optionally substituted with one R^{a}.

In some embodiments, R^{a} is C₃₋₈ cycloalkyl or C₁₋₆ alkyl.

In some embodiments, R^{a} is cyclopropyl or methyl.

In some embodiments, a compound as described herein has the structure of Formula IC or IIC

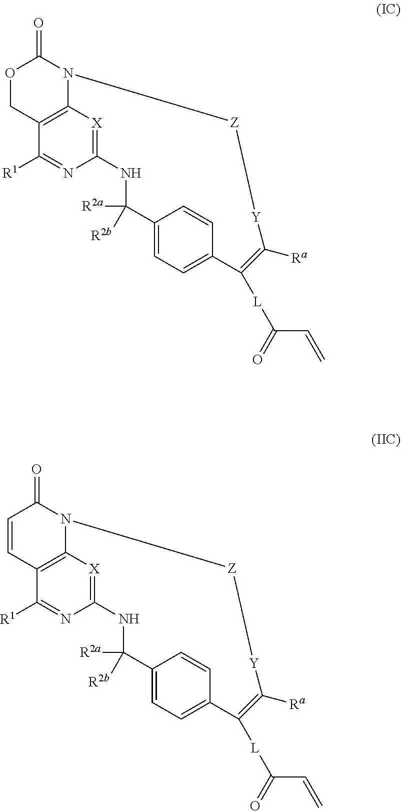

or a pharmaceutically acceptable salt thereof.

In some embodiments, the compound is a structure chosen from

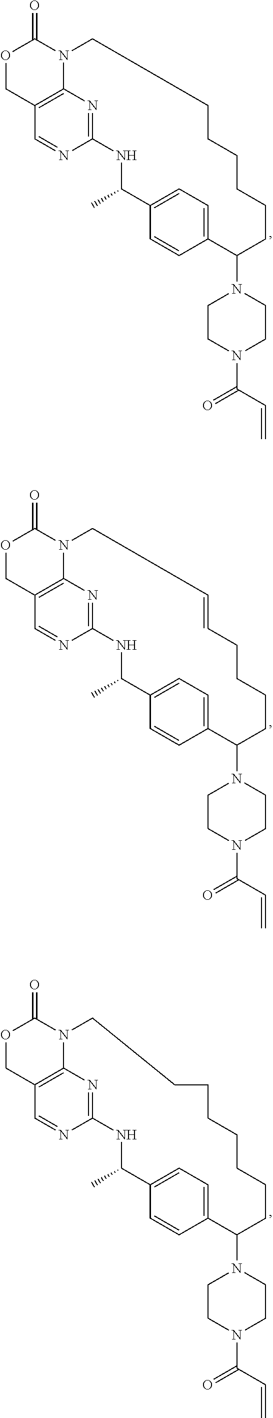 [SMILES: C=CC(=O)N1CCN(C2CCCCCCCN3C(=O)OCc4cnc(nc43)N[C@@H](C)c3ccc2cc3)CC1 ; C=CC(=O)N1CCN(C2CCCC/C=C/CN3C(=O)OCc4cnc(nc43)N[C@@H](C)c3ccc2cc3)CC1 ; C=CC(=O)N1CCN(C2CCCCCCCCN3C(=O)OCc4cnc(nc43)N[C@@H](C)c3ccc2cc3)CC1]

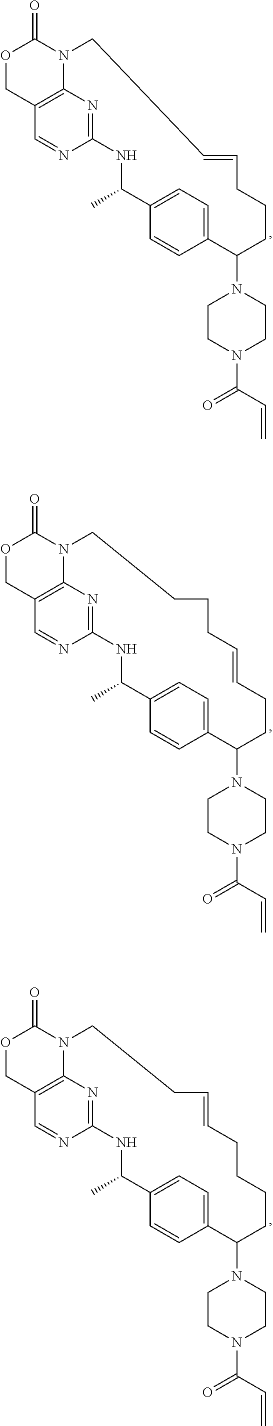 [SMILES: C=CC(=O)N1CCN(C2CCC/C=C/CN3C(=O)OCc4cnc(nc43)N[C@@H](C)c3ccc2cc3)CC1 ; C=CC(=O)N1CCN(C2CC/C=C/CCCCN3C(=O)OCc4cnc(nc43)N[C@@H](C)c3ccc2cc3)CC1 ; C=CC(=O)N1CCN(C2CCCC/C=C/CCN3C(=O)OCc4cnc(nc43)N[C@@H](C)c3ccc2cc3)CC1]

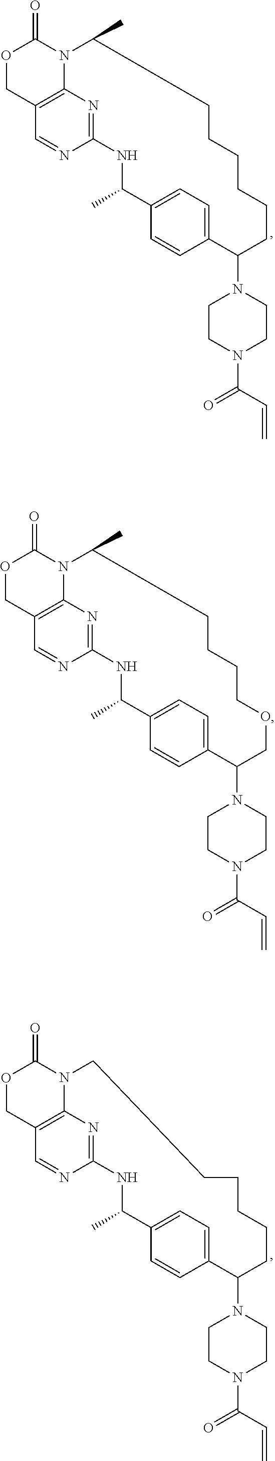 [SMILES: C=CC(=O)N1CCN(C2CCCCCC[C@H](C)N3C(=O)OCc4cnc(nc43)N[C@@H](C)c3ccc2cc3)CC1 ; C=CC(=O)N1CCN(C2COCCCC[C@H](C)N3C(=O)OCc4cnc(nc43)N[C@@H](C)c3ccc2cc3)CC1 ; C=CC(=O)N1CCN(C2CCCCCCN3C(=O)OCc4cnc(nc43)N[C@@H](C)c3ccc2cc3)CC1]

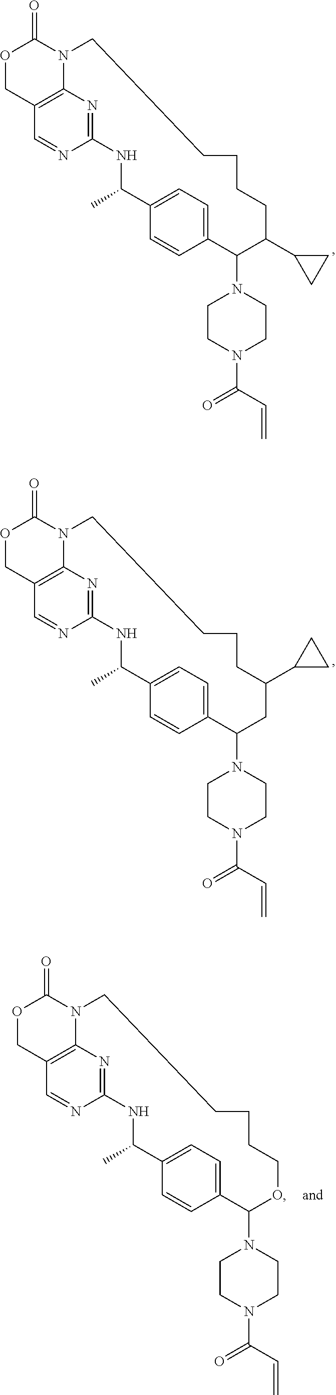 [SMILES: C=CC(=O)N1CCN(C2c3ccc(cc3)[C@H](C)Nc3ncc4c(n3)N(CCCCCC2C2CC2)C(=O)OC4)CC1 ; C=CC(=O)N1CCN(C2CC(C3CC3)CCCCN3C(=O)OCc4cnc(nc43)N[C@@H](C)c3ccc2cc3)CC1 ; C=CC(=O)N1CCN(C2OCCCCCN3C(=O)OCc4cnc(nc43)N[C@@H](C)c3ccc2cc3)CC1]

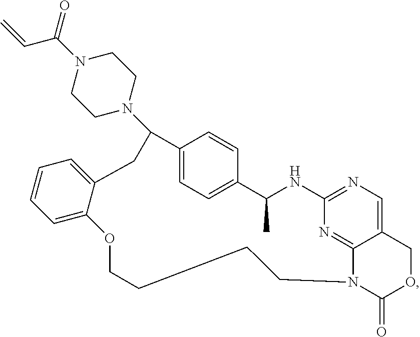 [SMILES: C=CC(=O)N1CCN(C2Cc3ccccc3OCCCCN3C(=O)OCc4cnc(nc43)N[C@@H](C)c3ccc2cc3)CC1]

or a pharmaceutically acceptable salt thereof.

The compounds disclosed herein can exist as pharmaceutically acceptable salts. The present disclosure includes compounds listed herein in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “pharmaceutically acceptable salt,” as used herein, represents salts or zwitterionic forms of the compounds disclosed herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form pharmaceutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

While it may be possible for the compounds, and pharmaceutically acceptable salts thereof, of the subject disclosure to be administered as the raw chemical, it is also possible to present them as a pharmaceutical formulation.

Also provided is a pharmaceutical formulation comprising a compound as disclosed herein, or a pharmaceutically acceptable salt thereof, together with a pharmaceutically acceptable carrier. The formulations may conveniently be presented in unit dosage form and may be prepared by any of the methods well known in the art of pharmacy. Typically, these methods include the step of bringing into association a compound, or pharmaceutically acceptable salts thereof, of the subject disclosure or a pharmaceutically acceptable salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Preferred unit dosage formulations are those containing an effective dose, or an appropriate fraction thereof, of the active ingredient.

Compounds, or pharmaceutically acceptable salts thereof, may be administered at a dose of from 0.1 to 500 mg/kg per day. The dose range for adult humans is generally from 5 mg to 2 g/day. The amount of active ingredient that may be combined with the carrier materials to produce a single dosage form will vary depending upon the host treated and the particular mode of administration.

The compounds, or pharmaceutically acceptable salts thereof, can be administered in various modes. In some embodiments, the pharmaceutical formulation is formulated for oral administration.

In certain instances, it may be appropriate to administer at least one of the compounds described herein (or a pharmaceutically acceptable salt thereof) in combination with another therapeutic agent. By way of example only, if one of the side effects experienced by a patient upon receiving one of the compounds herein, or pharmaceutically acceptable salt thereof, is hypertension, then it may be appropriate to administer an anti-hypertensive agent in combination with the initial therapeutic agent. Or, by way of example only, the therapeutic effectiveness of one of the compounds described herein, or pharmaceutically acceptable salts thereof, may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein, or pharmaceutically acceptable salts thereof, with another therapeutic agent (which also includes a therapeutic regimen) that also has therapeutic benefit. In any case, regardless of the disease, disorder or condition being treated, the overall benefit experienced by the patient may simply be additive of the two therapeutic agents or the patient may experience a synergistic benefit.

In any case, the multiple therapeutic agents (at least one of which is a compound disclosed herein, or a pharmaceutically acceptable salt thereof) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Also provided is a method for treating a disease or condition that benefits from or is treatable by inhibition of mutant IDH, comprising the administration of a therapeutically effective amount of a compound described herein, or a pharmaceutically acceptable salt thereof, to a subject in need thereof.

In some embodiments, the IDH protein is chosen from IDH1 and IDH2.

In some embodiments, the IDH protein is IDH1 and the one or more neomorphic mutations are found at residues chosen from R132 and S280.

In some embodiments, the neomorphic mutation is R132H.

In some embodiments, the neomorphic mutations are R132H and S280F.

In some embodiments, the IDH protein is IDH2 and the one or more neomorphic mutations are found at residue R140.

In some embodiments, the neomorphic mutation is R140Q.

Also provided are methods of inhibiting at least one mutant IDH function comprising the step of contacting mutant IDH with a compound as described herein, or a pharmaceutically acceptable salt thereof. The cell phenotype, cell proliferation, activity of mutant IDH, change in biochemical output produced by active mutant IDH, expression of mutant IDH, or binding of mutant IDH with a natural binding partner may be monitored. Such methods may be modes of treatment of disease, biological assays, cellular assays, biochemical assays, or the like.

Also provided are methods of treatment of an mutant IDH-mediated disease comprising the administration of a therapeutically effective amount of a compound as disclosed herein, or a pharmaceutically acceptable salt thereof, to a patient in need thereof.

Also provided is a method of inhibition of mutant IDH comprising contacting mutant IDH with a compound as disclosed herein, or a pharmaceutically acceptable salt thereof.

Also provided is a method of modulation of an mutant IDH-mediated function in a subject comprising the administration of a therapeutically effective amount of a compound as disclosed herein, or a pharmaceutically acceptable salt thereof.

In some embodiments, the mutant IDH-mediated disease is cancer.

In some embodiments, the cancer is chosen from Melanoma, Malignant Solid Tumors, Colorectal Carcinoma, Non-Small Cell Lung Carcinoma, Acute Myeloid Leukemia, Myelodysplastic Syndromes, Chronic Myelomonocytic Leukemia, Colorectal Adenocarcinoma, Multiple Myeloma, Non-Hodgkin Lymphoma, Pancreatic Carcinoma, Cutaneous Melanoma, Ovarian Carcinoma, Pancreatic Ductal Adenocarcinoma, Acute Lymphoblastic Leukemia, Thyroid Gland Carcinoma, Glioma, Neurofibromatosis, Poorly Differentiated Thyroid Gland Carcinoma, Myelodysplastic Syndrome With Excess Blasts, Juvenile Myelomonocytic Leukemia, Histiocytic And Dendritic Cell Neoplasm, Head And Neck Squamous Cell Carcinoma, Small Cell Lung Carcinoma, Low Grade Glioma, Squamous Cell Lung Carcinoma, Breast Carcinoma, Chronic Myelomonocytic Leukemia, Thyroid Gland Undifferentiated (Anaplastic) Carcinoma, Embryonal Rhabdomyosarcoma, Thyroid Gland Follicular Carcinoma, T-Cell Acute Lymphoblastic Leukemia, Mucosal Melanoma, Low Grade Ovarian Serous Adenocarcinoma, Thyroid Gland Papillary Carcinoma, Refractory Anemia With Excess Blasts, Myeloid Neoplasm, Myelodysplastic/Myeloproliferative Neoplasm, Rectal Carcinoma, Colon Carcinoma, Malignant Peripheral Nerve Sheath Tumor, Cholangiocarcinoma, Endometrial Carcinoma, Mantle Cell Lymphoma, Secondary Myelodysplastic Syndrome, Therapy-Related Myelodysplastic Syndrome, Lymphoma, Neuronal And Mixed Neuronal-Glial Tumors, Ganglioglioma, Soft Tissue Sarcoma, Bladder Carcinoma, Esophageal Carcinoma, Sarcoma, Thymic Carcinoma, Lung Adenocarcinoma, Lung Carcinoma, Uveal Melanoma, Head And Neck Carcinoma, Diffuse Glioma, Squamous Cell Carcinoma, Chronic Myeloid Leukemia, Adenocarcinoma of the Gastroesophageal Junction, Glioblastoma, Neuroblastoma, Astrocytic Tumor, Hepatocellular Carcinoma, Pancreatic Adenocarcinoma, Diffuse Large B-Cell Lymphoma, Anaplastic Astrocytoma, Gastric Adenocarcinoma, Gastric Carcinoma, Prostate Carcinoma, Renal Cell Carcinoma, B-Cell Acute Lymphoblastic Leukemia, Double-Hit Lymphoma, Dysembryoplastic Neuroepithelial Tumor, Gangliocytoma, Low-Grade Neuroepithelial Tumor, Peripheral T-Cell Lymphoma, Pilocytic Astrocytoma, Pilomyxoid Astrocytoma, Rhabdoid Tumor, and Schwannoma.

In some embodiments, the cancer is Acute Myeloid Leukemia.

In some embodiments, the cancer is chosen from Glioma, Diffuse Glioma, Ganglioglioma, and Low Grade Glioma.

Further embodiments include the embodiments disclosed in the following Schemes and Examples, which are not to be construed as limiting in any way.

In the Examples below and throughout the disclosure, the following abbreviations may be used: RT=Room Temperature; SM=Starting Material; MeCN or ACN=acetonitrile; CDI=1,1′-carbonyldiimidazole; DCE=dichloroethane; DCM=dichloromethane; DIEA or DIPEA=N,N-Diisopropylethylamine; DMF=dimethylformamide; DMSO=dimethylsulfoxide; Et₃N or TEA=triethylamine; EtOAc=ethyl acetate; EtOH=ethanol; H₂O=water; MeCN=acetonitrile; MeOH=methanol; n-BuLi=n-butyllithium; NMP=N-methyl-2-pyrrolidone; PE=petroleum ether; TFA=trifluoroacetic acid; TFAA=trifluoroacetic anhydride; THF=tetrahydrofuran; ¹H-NMR=Proton Nuclear magnetic Resonance; LCMS=Liquid chromatography-mass spectrometry; TLC=thin layer chromatography; and HPLC=High Performance Liquid Chromatography. Other abbreviations may be used and will be familiar in context to those of skill in the art.

### Schemes

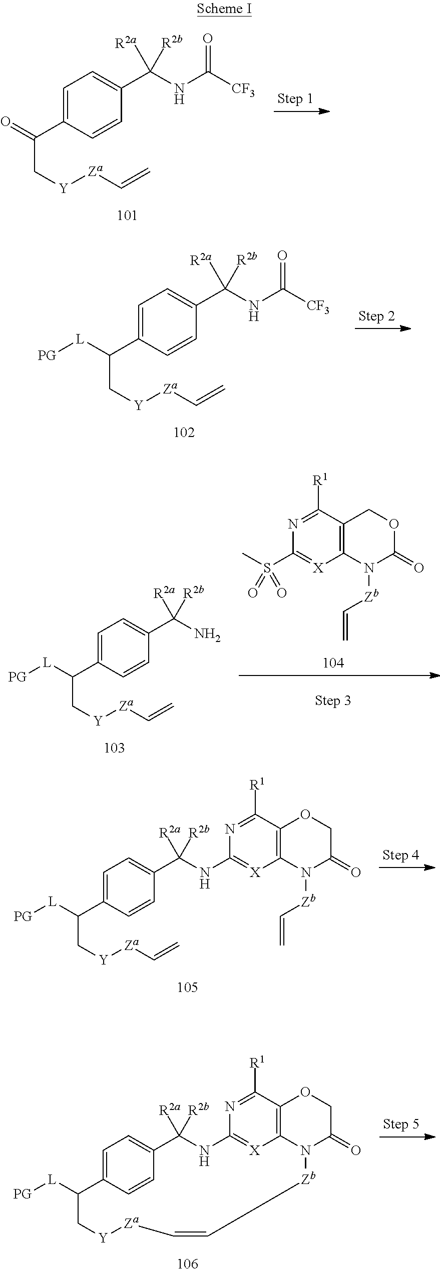

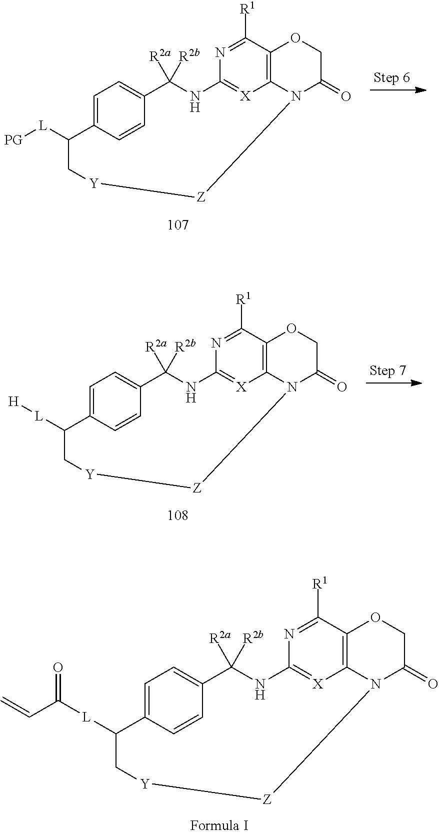

Referring to Scheme I, Step 1, to a solution of a compound of Formula 101 (Z^{a}=a saturated or partially unsaturated bivalent hydrocarbon chain optionally substituted with one or more R^{a}, wherein 1 or 2 nonconsecutive methylene units of the chain may be independently replaced by —O—, —OC(O)—, —C(O)O—, —C(O)—, —NR^{b}C(O)—, —C(O)NR^{b}—, —OC(O)NR^{b}—, or —NR^{b}C(O)O—) in a polar aprotic solvent, such as THF, is added a compound of formula H-L-PG (PG=protecting group, such as tert-butyloxycarbonyl), and a Lewis acid, such as Titanium isopropoxide. The mixture is stirred, optionally at elevated temperature. In some embodiments, the mixture is stirred from 12-24 h. To the mixture is added a reducing agent, such as sodium cyanoborohydride, and a polar protic solvent, such as methanol. The mixture is further stirred, optionally at ambient temperature. In some embodiments, the mixture is further stirred for 1-4 h. The product, a compound of Formula 102, is isolated and purified using methods known in the art.

Referring to Scheme I, Step 2, to a solution of the compound of Formula 102 in a polar solvent, such as ethanol, is added a strong base, such as potassium hydroxide. The mixture is stirred, optionally at ambient temperature. In some embodiments, the mixture is stirred for 12-36 h. The product, a compound of Formula 103, is isolated and purified using methods known in the art.

Referring to Scheme I, Step 3, to a solution of a compound of Formula 103 in a polar aprotic solvent, such as dimethylsulfoxide, is added a fluoride source, such as cesium fluoride, a compound of Formula 104 (Z^{b}=a saturated or partially unsaturated bivalent hydrocarbon chain optionally substituted with one or more R^{a}, wherein 1 or 2 nonconsecutive methylene units of the chain may be independently replaced by —O—, —OC(O)—, —C(O)O—, —C(O)—, —NR^{b}C(O)—, —C(O)NR^{b}—, —OC(O)NR^{b}—, or —NR^{b}C(O)O—), and a non-nucleophilic base, such as N,N-diisopropylethylamine. The mixture is stirred, optionally at elevated temperatures. In some embodiments, the mixture is stirred for 1-3 h. The product, a compound of Formula 105, is isolated and purified using methods known in the art.

Referring to Scheme I, Step 4, a solution of a compound of Formula 105 in a polar organic solvent, such as trifluoroacetic acid (TFA), is added an olefin metathesis catalyst, such as Hoveyda-Grubbs II. The mixture is stirred, optionally at elevated temperatures. In some embodiments, the mixture is stirred for 1-3 h. The product, a compound of Formula 106, is isolated and purified using methods known in the art. In some embodiments, the compound of Formula 106 is also a compound of Formula 107 and is carried through to Step 6 of Scheme I.

Referring to Scheme I, Step 5, a solution of a compound of Formula 106 in an organic solvent, such as dichloromethane, is added a reducing agent, such as palladium on carbon, under a hydrogen gas environment. The mixture is stirred, optionally at ambient temperature. In some embodiments, the mixture is stirred for 1-3 h. The product, a compound of Formula 107, is isolated and purified using methods known in the art.

Referring to Scheme I, Step 6, a solution of a compound of Formula 107 in an organic solvent, such as dioxane, is subjected to any set of deprotection conditions known in the art. The product, a compound of Formula 108, is isolated and purified using methods known in the art.

Referring to Scheme I, Step 7, a solution of a compound of Formula 108 in an organic solvent, such as dichloromethane, is added prop-2-enoyl chloride. The mixture is stirred, optionally at reduced temperatures. In some embodiments, the mixture is stirred for 5-10 min. The product, a compound of Formula I, is isolated and purified using methods known in the art. Individual enantiomers can be separated by using methods known in the art, such as chiral chromatography.

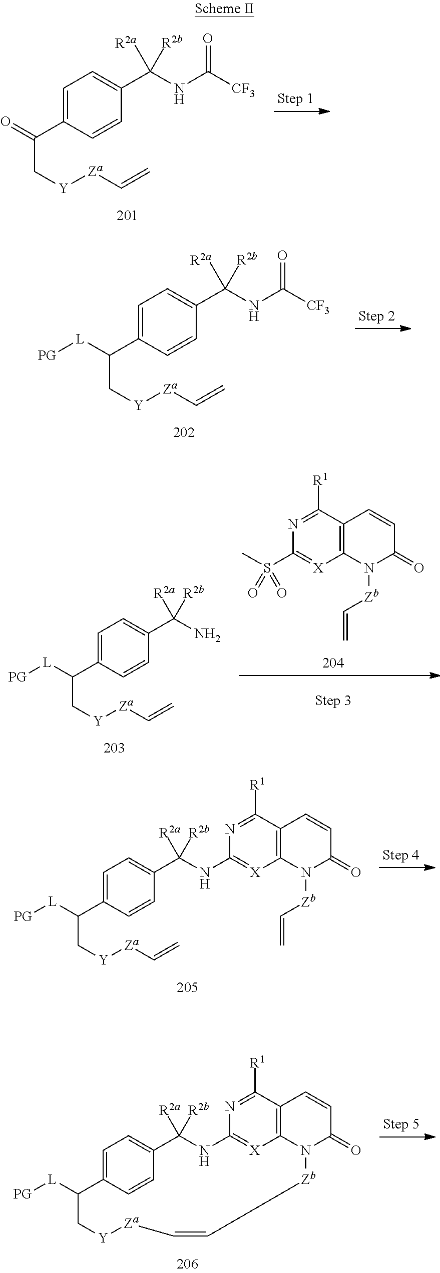

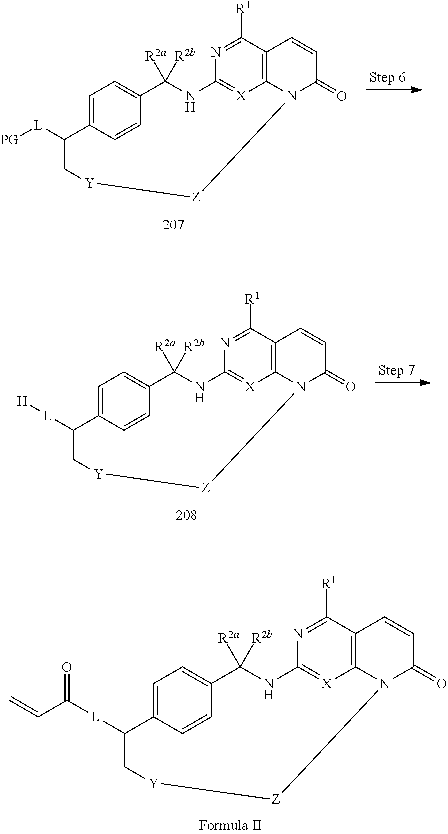

Referring to Scheme II, Step 1, to a solution of a compound of Formula 101 (Z^{a}=a saturated or partially unsaturated bivalent hydrocarbon chain optionally substituted with one or more R^{a}, wherein 1 or 2 nonconsecutive methylene units of the chain may be independently replaced by —O—, —OC(O)—, —C(O)O—, —C(O)—, —NR^{b}C(O)—, —C(O)NR^{b}—, —OC(O)NR^{b}—, or —NR^{b}C(O)O—) in a polar aprotic solvent, such as THF, is added a compound of formula H-L-PG (PG=protecting group, such as tert-butyloxycarbonyl), and a Lewis acid, such as Titanium isopropoxide. The mixture is stirred, optionally at elevated temperature. In some embodiments, the mixture is stirred from 12-24 h. To the mixture is added a reducing agent, such as sodium cyanoborohydride, and a polar protic solvent, such as methanol. The mixture is further stirred, optionally at ambient temperature. In some embodiments, the mixture is further stirred for 1-4 h. The product, a compound of Formula 102, is isolated and purified using methods known in the art.

Referring to Scheme II, Step 2, to a solution of the compound of Formula 102 in a polar solvent, such as ethanol, is added a strong base, such as potassium hydroxide. The mixture is stirred, optionally at ambient temperature. In some embodiments, the mixture is stirred for 12-36 h. The product, a compound of Formula 103, is isolated and purified using methods known in the art.

Referring to Scheme II, Step 3, to a solution of a compound of Formula 103 in a polar aprotic solvent, such as dimethylsulfoxide, is added a fluoride source, such as cesium fluoride, a compound of Formula 104 (Z^{b}=a saturated or partially unsaturated bivalent hydrocarbon chain optionally substituted with one or more R^{a}, wherein 1 or 2 nonconsecutive methylene units of the chain may be independently replaced by —O—, —OC(O)—, —C(O)O—, —C(O)—, —NR^{b}C(O)—, —C(O)NR^{b}—, —OC(O)NR^{b}—, or —NR^{b}C(O)O—), and a non-nucleophilic base, such as N,N-diisopropylethylamine. The mixture is stirred, optionally at elevated temperatures. In some embodiments, the mixture is stirred for 1-3 h. The product, a compound of Formula 105, is isolated and purified using methods known in the art.

Referring to Scheme II, Step 4, a solution of a compound of Formula 105 in a polar organic solvent, such as TFA, is added an olefin metathesis catalyst, such as Hoveyda-Grubb's II. The mixture is stirred, optionally at elevated temperatures. In some embodiments, the mixture is stirred for 1-3 h. The product, a compound of Formula 106, is isolated and purified using methods known in the art. In some embodiments, the compound of Formula 106 is also a compound of Formula 107 and is carried through to Step 6 of Scheme I.

Referring to Scheme II, Step 5, a solution of a compound of Formula 106 in an organic solvent, such as dichloromethane, is added a reducing agent, such as palladium on carbon, under a hydrogen gas environment. The mixture is stirred, optionally at ambient temperature. In some embodiments, the mixture is stirred for 1-3 h. The product, a compound of Formula 107, is isolated and purified using methods known in the art.

Referring to Scheme II, Step 6, a solution of a compound of Formula 107 in an organic solvent, such as dioxane, is subjected to any set of deprotection conditions known in the art. The product, a compound of Formula 108, is isolated and purified using methods known in the art.

Referring to Scheme II, Step 7, a solution of a compound of Formula 108 in an organic solvent, such as dichloromethane, is added prop-2-enoyl chloride. The mixture is stirred, optionally at reduced temperatures. In some embodiments, the mixture is stirred for 5-10 min. The product, a compound of Formula I, is isolated and purified using methods known in the art. Individual enantiomers can be separated by using methods known in the art, such as chiral chromatography.

### Intermediate A

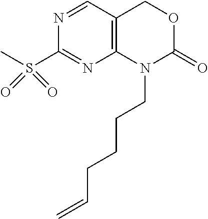 [SMILES: C=CCCCCN1C(=O)OCc2cnc(S(C)(=O)=O)nc21]

### 1-Hex-5-enyl-7-methylsulfonyl-4H-pyrimido[4,5-d][1,3]oxazin-2-one

### Step 1: 7-Methylsulfanyl-1,4-dihydropyrimido[4,5-d][1,3]oxazin-2-one

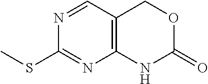

To a solution of (4-amino-2-methylsulfanyl-pyrimidin-5-yl) methanol (10 g, 58.4 mmol, 1 eq) in THF (200 mL) was added triphosgene (9.57 g, 32.2 mmol, 0.55 eq) at −30° C., DIPEA (36.6 mL, 210 mmol, 3.6 eq) was added dropwise while maintaining the reaction temperature between −35° C. and −30° C. The mixture was stirred at −30° C. for 1 h. LCMS showed starting material was consumed completely. The reaction mixture was poured into ice water (300 mL) and extracted with ethyl acetate (150 mL×3). The combined organic layers were dried over Na₂SO₄, filtered and concentrated under reduced pressure to afford 7-methylsulfanyl-1,4-dihydropyrimido[4,5-d][1,3]oxazin-2-one (12 g, 51.1 mmol, 87.5% yield, 84% purity) as a yellow solid.

MS (ES⁺) C₇H₇N₃O₂S requires: 197, found: 198 [M+H]⁺,

### Step 2: 1-Hex-5-enyl-7-methylsulfanyl-4H-pyrimido[4,5-d][1,3]oxazin-2-one

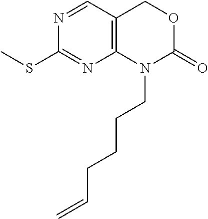 [SMILES: C=CCCCCN1C(=O)OCc2cnc(SC)nc21]

To a solution of 7-methylsulfanyl-1,4-dihydropyrimido[4,5-d][1,3]oxazin-2-one (1.8 g, 9.13 mmol, 1.0 eq) in NMP (18 mL) was added K₂CO₃ (3.78 g, 27.4 mmol, 3.0 eq) and 6-bromohex-1-ene (2.44 mL, 18.3 mmol, 2.0 eq). The mixture was stirred at 60° C. for 16 h under N₂ atmosphere. LCMS showed starting material was consumed completely. The reaction mixture was diluted with EtOAc (40 mL) and washed with brine (30 mL×3), dried over Na₂SO₄, filtered and concentrated under reduced pressure to afford a residue which was purified by flash silica gel chromatography (20 g SepaFlash® Silica Flash Column, Eluent of 0-15% Ethyl acetate/Petroleum ether gradient @80 mL/min) to afford 1-hex-5-enyl-7-methylsulfanyl-4H-pyrimido[4,5-d][1,3]oxazin-2-one (1.5 g, 5.32 mmol, 58.2% yield, 99% purity) as yellow oil. MS (ES⁺) C₁₃H₁₇N₃O₂S requires: 279, found: 280 [M+H]⁺.

### Step 3: 1-hex-5-enyl-7-methylsulfonyl-4H-pyrimido[4,5-d][1,3]oxazin-2-one

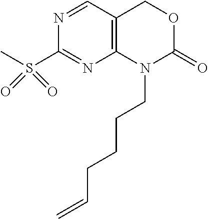 [SMILES: C=CCCCCN1C(=O)OCc2cnc(S(C)(=O)=O)nc21]

To a solution of 1-hex-5-enyl-7-methylsulfanyl-4H-pyrimido[4,5-d][1,3]oxazin-2-one (1.16 g, 4.15 mmol, 1.0 eq) in ACN (21 mL) and H₂O (10 mL) was added Oxone (5.11 g, 8.30 mmol, 2.0 eq) portion wise. The mixture was stirred at 20° C. for 16 h. LCMS showed starting material was consumed completely. The reaction mixture was quenched by addition saturated Na₂SO₃ (50 mL), the mixture was stirred at 20° C. for 0.5 h and extracted with EtOAc (50 mL×3), dried over Na₂SO₄, filtered and concentrated under reduced pressure to afford a residue which was purified by flash silica gel chromatography (20 g SepaFlash® Silica Flash Column, Eluent of 0-50% Ethyl acetate/Petroleum ether gradient @60 mL/min) to afford 1-hex-5-enyl-7-methylsulfonyl-4H-pyrimido[4,5-d][1,3]oxazin-2-one (711 mg, 2.28 mmol, 55.0% yield) as white solid. MS (ES⁺) C₁₃H₁₇N₃O₄S requires: 311, found: 312 [M+H]⁺. ¹H NMR (400 MHz, DMSO-d₆) δ=8.68 (s, 1H), 5.90-5.75 (m, 1H), 5.49 (s, 2H), 5.10-4.86 (m, 2H), 3.96 (t, J=7.4 Hz, 2H), 3.40 (s, 3H), 2.10-2.01 (m, 2H), 1.69-1.59 (m, 2H), 1.45-1.35 (m, 2H).

### Intermediate B

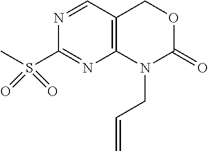 [SMILES: C=CCN1C(=O)OCc2cnc(S(C)(=O)=O)nc21]

### 1-allyl-7-(methylsulfonyl)-1,4-dihydro-2H-pyrimido[4,5-d][1,3]oxazin-2-one

Made similarly to Intermediate A. ¹H NMR (600 MHz, CDCl₃) δ 8.52 (s, 1H), 5.93 (ddt, J=16.3, 10.2, 6.0 Hz, 1H), 5.41 (s, 2H), 5.38 (dd, J=17.2, 1.4 Hz, 1H), 5.28 (dt, J=10.3, 1.1 Hz, 1H), 4.75-4.70 (m, 2H), 3.32 (s, 3H).

### Intermediate C

### N-[(1S)-1-(4-bromophenyl)ethyl]-2,2,2-trifluoro-acetamide

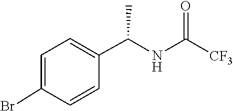 [SMILES: C[C@H](NC(=O)C(F)(F)F)c1ccc(Br)cc1]

To a solution of (1S)-1-(4-bromophenyl)ethanamine (14.2 g, 71.0 mmol, 10.2 mL, 1.0 eq) in ACN (150 mL) was added TFAA (10.9 mL, 78.1 mmol, 1.1 eq) and TEA (21.7 mL, 156 mmol, 2.2 eq). The mixture was stirred at 5° C. for 1.5 hr under N₂. TLC (Petroleum ether:Ethyl acetate=3:1) showed material was consumed completely. To the mixture was added water (200 mL) and extracted with EtOAc (200 mL×3), dried over Na₂SO₄, filtered and concentrated under reduced pressure to give a residue which was purified by flash silica gel chromatography (220 g SepaFlash® Silica Flash Column, Eluent of 0-10% Ethyl acetate/Petroleum ether gradient @200 mL/min) to afford N-[(1S)-1-(4-bromophenyl)ethyl]-2,2,2-trifluoro-acetamide (20.6 g, 69.4 mmol, 97.7% yield) as a white solid. ¹H NMR (400 MHz, DMSO-d₆) δ=9.90 (d, J=6.4 Hz, 1H), 7.55 (d, J=8.4 Hz, 2H), 7.30 (d, J=8.3 Hz, 2H), 5.05-4.90 (m, 1H), 1.44 (d, J=7.1 Hz, 3H).

### Example 1

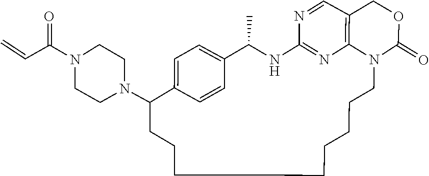 [SMILES: C=CC(=O)N1CCN(C2CCCCCCCCN3C(=O)OCc4cnc(nc43)N[C@@H](C)c3ccc2cc3)CC1]

### (3S)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacyclotridecaphan-1²-one

### Step 1: N-methoxy-N-methyl-pent-4-enamide

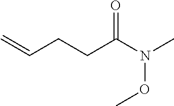

A suspension of CDI (24.3 g, 150. mmol, 1.0 eq) in DCM (70 mL) was added a solution of pent-4-enoic acid (15 g, 150 mmol, 15.3 mL, 1.0 eq) in DCM (70 mL). The mixture was stirred for 15 min at 20° C., to the mixture was added N-methoxymethanamine (14.6 g, 150. mmol, 1.0 eq, HCl) and TEA (30.3 g, 300 mmol, 41.7 mL, 2.0 eq). The mixture was stirred at 20° C. for another 2 h. TLC (Petroleum ether:Ethyl acetate=2:1) showed starting material was consumed. The reaction mixture was washed with saturated citric acid (100 mL×3) and saturated NaHCO₃ (100 mL×3), dried over Na₂SO₄, filtered and concentrated under reduced pressure to give N-methoxy-N-methyl-pent-4-enamide (20 g, 140 mmol, 93.2% yield) as colorless oil. ¹H NMR (400 MHz, CDCl₃) δ=5.90-5.70 (m, 1H), 5.13-5.02 (m, 1H), 5.01-4.93 (m, 1H), 3.68 (s, 3H), 3.18 (s, 3H), 2.56-2.48 (m, 2H), 2.42-2.35 (m, 2H).

### Step 2: 2,2,2-Trifluoro-N-[(1S)-1-(4-pent-4-enoylphenyl)ethyl]acetamide

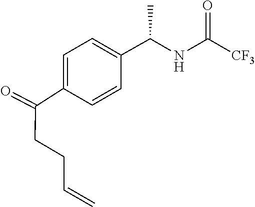 [SMILES: C=CCCC(=O)c1ccc([C@H](C)NC(=O)C(F)(F)F)cc1]

To a solution of N-[(1S)-1-(4-bromophenyl)ethyl]-2,2,2-trifluoro-acetamide (8.1 g, 27.4 mmol, 1.0 eq) in THF (120 mL) was added n-BuLi (2 M in heptane, 27.4 mL, 54.8 mmol, 2.0 eq) dropwise at −78° C. under the protection of N₂. The mixture was stirred at −78° C. for 45 min and then a solution of N-methoxy-N-methyl-pent-4-enamide (3.92 g, 27.36 mmol, 1 eq) in THF (12 mL) was added to the stirring mixture at −78° C. The resulting mixture was stirred at −78° C. for another 45 min. TLC (Petroleum ether:Ethyl acetate=3:1) showed N-[(1S)-1-(4-bromophenyl)ethyl]-2,2,2-trifluoro-acetamide was consumed completely. The reaction mixture was quenched with saturated NH₄Cl (200 mL) and extracted with EtOAc (200 mL×3), dried over Na₂SO₄, filtered and concentrated under reduced pressure to give a residue which was purified by flash silica gel chromatography (40 g SepaFlash® Silica Flash Column, Eluent of 0-10% Ethyl acetate/Petroleum ether gradient @100 mL/min) to afford 2,2,2-trifluoro-N-[(1S)-1-(4-pent-4-enoylphenyl)ethyl]acetamide (2.6 g, 8.69 mmol, 31.7% yield) as a white solid. ¹H NMR (400 MHz, CDCl₃) δ=7.97 (d, J=8.2 Hz, 2H), 7.42 (d, J=8.2 Hz, 2H), 6.64 (d, J=4.3 Hz, 1H), 5.98-5.75 (m, 1H), 5.25-5.20 (m, 1H), 5.13-5.00 (m, 2H), 3.07 (t, J=7.4 Hz, 2H), 2.55-2.44 (m, 2H), 1.61 (d, J=7.0 Hz, 3H).

### Step 3: Tert-butyl 4-[1-[4-[(1S)-1-[(2,2,2-trifluoroacetyl)amino]ethyl]phenyl]pent-4-enyl]piperazine-1-carboxylate

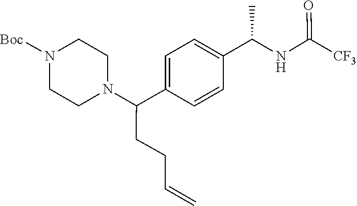 [SMILES: C=CCCC(c1ccc([C@H](C)NC(=O)C(F)(F)F)cc1)N1CCN(C(=O)OC(C)(C)C)CC1]

To a solution of 2,2,2-trifluoro-N-[(1S)-1-(4-pent-4-enoylphenyl)ethyl]acetamide (3 g, 10.0 mmol, 1.0 eq) in THF (60 mL) was added tetraisopropoxytitanium (14.8 mL, 50.1 mmol, 5.0 eq) and tert-butyl piperazine-1-carboxylate (3.73 g, 20.1 mmol, 2.0 eq), the mixture was stirred at 60° C. for 16 h. to the mixture was added NaBH₃CN (1.26 g, 20.1 mmol, 2 eq) and MeOH (60 mL). The mixture was stirred at 20° C. for another 3 h. LCMS showed starting material was consumed completely. The reaction mixture was quenched with saturated NaHCO₃ (200 mL) and extracted with EtOAc (200 mL×3), dried over Na₂SO₄, filtered and concentrated under reduced pressure to give a residue which was purified by flash silica gel chromatography (40 g SepaFlash® Silica Flash Column, Eluent of 0-25% Ethyl acetate/Petroleum ether gradient @80 mL/min) to afford tert-butyl 4-[1-[4-[(1S)-1-[(2,2,2-trifluoroacetyl)amino]ethyl]phenyl]pent-4-enyl]piperazine-1-carboxylate (1.5 g, 3.00 mmol, 30.0% yield, 94% purity) as a yellow oil and the other impure portion was purified by prep-HPLC (column: Phenomenex luna C18 150*40 mm*15 μm; mobile phase: [water(FA)-ACN]; gradient: 22%-52% B over 15 min) to afford tert-butyl 4-[1-[4-[(1S)-1-[(2,2,2-trifluoroacetyl)amino]ethyl]phenyl]pent-4-enyl]piperazine-1-carboxylate (300 mg, 626 mol, 6.3% yield, 98% purity) as yellow oil. MS (ES⁺) C₂₄H₃₄N₃O₃F₃ requires: 469, found: 470 [M+H]⁺.

### Step 4: Tert-butyl 4-[1-[4-[(1S)-1-aminoethyl]phenyl]pent-4-enyl]piperazine-1-carboxylate

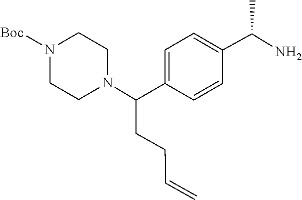 [SMILES: C=CCCC(c1ccc([C@H](C)N)cc1)N1CCN(C(=O)OC(C)(C)C)CC1]

To a solution of tert-butyl 4-[1-[4-[(1S)-1-[(2,2,2-trifluoroacetyl)amino]ethyl]phenyl]pent-4-enyl]piperazine-1-carboxylate (1.50 g, 3.19 mmol, 1.0 eq) in EtOH (100 mL) was added KOH (5 M in H₂O, 3.19 mL, 16.0 mmol, 5.0 eq), the mixture was stirred at 20° C. for 16 h. LCMS showed partial completion. To the mixture was added KOH (5 M, 20 mL, 100 mmol, 31.3 eq), the mixture was stirred at 30° C. for another 16 h. LCMS showed starting material was consumed completely. To the mixture was added water (150 mL) and extracted with EtOAc (150 mL×3), the combined organic phase was washed with brine (150 mL×3), dried over Na₂SO₄, filtered and concentrated under reduced pressure to afford tert-butyl 4-[1-[4-[(1S)-1-aminoethyl]phenyl]pent-4-enyl]piperazine-1-carboxylate (1.06 g) as yellow oil and was used without further purification. MS (ES⁺) C₂₂H₃₅N₃O₂ requires: 373, found: 374 [M+H]⁺.

### Step 5: Tert-butyl 4-[1-[4-[(1S)-1-[(1-hex-5-enyl-2-oxo-4H-pyrimido[4,5-d][1,3]oxazin-7-yl)amino]ethyl]phenyl]pent-4-enyl]piperazine-1-carboxylate

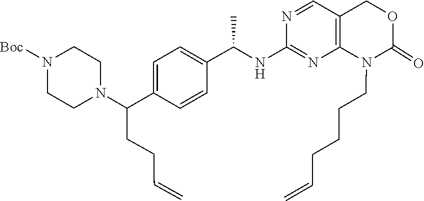 [SMILES: C=CCCCCN1C(=O)OCc2cnc(N[C@@H](C)c3ccc(C(CCC=C)N4CCN(C(=O)OC(C)(C)C)CC4)cc3)nc21]

To a solution of tert-butyl 4-[1-[4-[(1S)-1-aminoethyl]phenyl]pent-4-enyl]piperazine-1-carboxylate (1 g, 2.7 mmol, 1.0 eq) in DMSO (10 mL) was added CsF (1.22 g, 8.03 mmol, 3.0 eq), 1-hex-5-enyl-7-methylsulfonyl-4H-pyrimido[4,5-d][1,3]oxazin-2-one (834 mg, 2.68 mmol, 1.0 eq) and DIPEA (700 μL, 4.02 mmol, 1.5 eq). The mixture was stirred at 60° C. for 1.5 h. To the mixture was added water (20 mL) and extracted with ethyl acetate (20 mL×3), the combined organic phase was washed with brine (20 mL×3), dried over Na₂SO₄, filtered and concentrated under reduced pressure to give a residue which was purified by prep-HPLC (column: Phenomenex luna C18 150*25 mm*10 μm; mobile phase: [water(FA)-ACN]; gradient: 28%-58% B over 10 min) and lyophilized to afford tert-butyl 4-[1-[4-[(1S)-1-[(1-hex-5-enyl-2-oxo-4H-pyrimido[4,5-d][1,3]oxazin-7-yl)amino]ethyl]phenyl]pent-4-enyl]piperazine-1-carboxylate (1.1 g, 1.78 mmol, 66.5% yield, 98% purity) as a yellow oil. MS (ES⁺) C₃₄H₄₈N₆O₄ requires: 604, found: 605 [M+H]⁺.

### Step 6: Tert-butyl 4-((3S,Z)-3-methyl-1²-oxo-1¹,1⁴-dihydro-1²H-2-aza-1(7,1)-pyrimido[4,5-d][1,3]oxazina-4(1,4)-benzenacyclotridecaphan-8-en-5-yl)piperazine-1-carboxylate

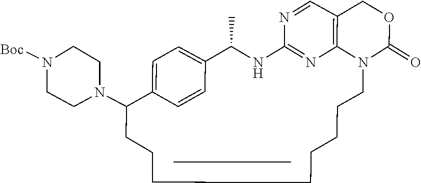

To a solution of tert-butyl 4-[1-[4-[(1S)-1-[(1-hex-5-enyl-2-oxo-4H-pyrimido[4,5-d][1,3]oxazin-7-yl)amino]ethyl]phenyl]pent-4-enyl]piperazine-1-carboxylate (200 mg, 331 μmol, 1.0 eq) in DCE (20 mL) was added Hoveyda-Grubbs II (44.4 mg, 71.0 mol, 0.21 eq). The mixture was stirred at 60° C. for 9 hr under N₂, the mixture was purged with N₂ several times every 1 hour. LCMS showed starting material was consumed completely. The reaction mixture was concentrated in vacuum and purified by flash silica gel chromatography (12 g SepaFlash® Silica Flash Column, Eluent of 0-100% Ethyl acetate/Petroleum ether to 0-50% DCM: Methanol gradient @80 mL/min) to afford the title compound (100 mg, 163 mol, 49.2% yield, 94% purity) as a green oil. MS (ES⁺) C₃₂H₄₄N₆O₄ requires: 576, found: 577 [M+H]⁺.

### Step 7: Tert-butyl 4-((3S)-3-methyl-1²-oxo-1¹,1⁴-dihydro-1²H-2-aza-1(7,1)-pyrimido[4,5-d][1,3]oxazina-4(1,4)-benzenacyclotridecaphane-5-yl)piperazine-1-carboxylate

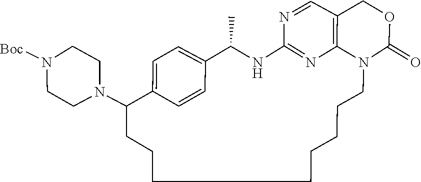 [SMILES: C[C@@H]1Nc2ncc3c(n2)N(CCCCCCCCC(N2CCN(C(=O)OC(C)(C)C)CC2)c2ccc1cc2)C(=O)OC3]

To a solution of tert-butyl 4-((3S,Z)-3-methyl-1²-oxo-1¹,1⁴-dihydro-1²H-2-aza-1(7,1)-pyrimido[4,5-d][1,3]oxazina-4(1,4)-benzenacyclotridecaphan-8-en-5-yl)piperazine-1-carboxylate (40 mg, 69.4 mol, 1.0 eq) in CF₃CH₂OH (6 mL) was added Pd/C (22.1 mg, 20.8 μmol, 10% purity, 0.3 eq) under N₂. The suspension was degassed under vacuum and purged with H₂ several times. The mixture was stirred at 20° C. for 2 h under H₂ (15 psi). LCMS showed starting material was consumed completely. The reaction mixture was filtered and concentrated in vacuum to afford the title compound (21.7 mg, 35.3 mol, 50.8% yield, 94% purity) as a green oil. MS (ES⁺) C₃₂H₄₆N₆O₄ requires: 578, found: 579 [M+H]⁺.

### Step 8: (3S)-3-methyl-5-(piperazin-1-yl)-1¹,1⁴-dihydro-1²H-2-aza-1(7,1)-pyrimido[4,5-d][1,3]oxazina-4(1,4)-benzenacyclotridecaphan-1²-one

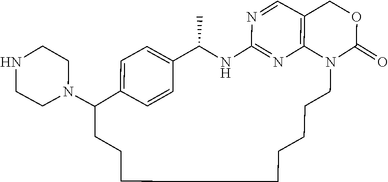 [SMILES: C[C@@H]1Nc2ncc3c(n2)N(CCCCCCCCC(N2CCNCC2)c2ccc1cc2)C(=O)OC3]

To a solution of tert-butyl 4-((3S)-3-methyl-1²-oxo-1¹,1⁴-dihydro-1²H-2-aza-1(7,1)-pyrimido[4,5-d][1,3]oxazina-4(1,4)-benzenacyclotridecaphane-5-yl)piperazine-1-carboxylate (21.7 mg, 37.5 mol, 1.0 eq) in dioxane (1 mL) was added HCl/dioxane (5 M, 995 μL). The mixture was stirred at 20° C. for 1 hr. LCMS showed starting material was consumed completely. The reaction mixture was quenched with NaHCO₃ (10 mL) and extracted with EtOAc (10 mL×3), dried over Na₂SO₄, filtered and the filtrate was concentrated in vacuum to afford the title compound (20 mg crude) as a yellow oil and was taken forward without further purification. MS (ES⁺) C₂₇H₃₈N₆O₂ requires: 478, found: 479 [M+H]⁺.

### Step 9: (3S)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacyclotridecaphan-1²-one

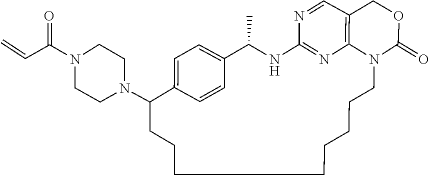 [SMILES: C=CC(=O)N1CCN(C2CCCCCCCCN3C(=O)OCc4cnc(nc43)N[C@@H](C)c3ccc2cc3)CC1]

To a solution of prop-2-enoyl chloride (3.73 μL, 46 mol, 1.1 eq) in DCM (0.5 mL) was added a solution of (3S)-3-methyl-5-(piperazin-1-yl)-1¹,1⁴-dihydro-1²H-2-aza-1(7,1)-pyrimido[4,5-d][1,3]oxazina-4(1,4)-benzenacyclotridecaphan-1²-one (20 mg, crude) in DCM (1 mL) dropwise at −78° C. After 2 min at −78° C. a few drops of methanol was added, followed by saturated NaHCO₃ (2 mL). The mixture was allowed to warm to 20° C. LCMS showed starting material was consumed completely. To the mixture was added water (8 mL) and extracted with EtOAc (8 mL×3), dried over Na₂SO₄, filtered and concentrated under reduced pressure to give a residue which was purified by prep-HPLC (column: Phenomenex luna C18 150*25 mm*10 μm; mobile phase: [water(FA)-ACN]; gradient: 10%-30% B over 25 min) and lyophilized to afford (3S)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacyclotridecaphan-1²-one (1.6 mg, 2.76 mol, 6.6% yield, 100% purity, FA) as a white solid. MS (ES⁺) C₃₁H₄₂N₆O₅ requires: 532, found: 533 [M+H]⁺. ¹H NMR (400 MHz, CD₃OD) δ=8.51-8.33 (m, 1H), 7.99 (s, 1H), 7.46-7.21 (m, 4H), 6.79-6.62 (m, 1H), 6.16 (d, J=17.0 Hz, 1H), 5.82-5.60 (m, 1H), 5.23-5.01 (m, 3H), 4.27-4.01 (m, 1H), 3.79-3.50 (m, 5H), 2.66-2.32 (m, 4H), 2.07-1.78 (m, 2H), 1.50 (d, J=7.0 Hz, 3H), 1.37-0.60 (m, 13H).

### Example 2

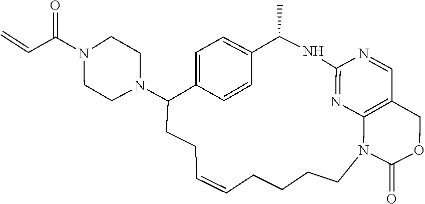 [SMILES: C=CC(=O)N1CCN(C2CC/C=C\CCCCN3C(=O)OCc4cnc(nc43)N[C@@H](C)c3ccc2cc3)CC1]

### (3S,Z)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacyclotridecaphan-8-en-1²-one

### Step 1: (3S,Z)-3-methyl-5-(piperazin-1-yl)-1¹,1⁴-dihydro-1²H-2-aza-1(7,1)-pyrimido[4,5-d][1,3]oxazina-4(1,4)-benzenacyclotridecaphan-8-en-1²-one

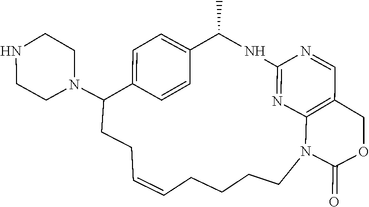 [SMILES: C[C@@H]1Nc2ncc3c(n2)N(CCCC/C=C\CCC(N2CCNCC2)c2ccc1cc2)C(=O)OC3]

To a solution of tert-butyl 4-((3S,Z)-3-methyl-1²-oxo-1¹,1⁴-dihydro-1²H-2-aza-1(7,1)-pyrimido[4,5-d][1,3]oxazina-4(1,4)-benzenacyclotridecaphan-8-en-5-yl)piperazine-1-carboxylate (40 mg, 69.4 mol, 1.0 eq) in dioxane (1 mL) was added HCl/dioxane (4 M, 1 mL). The mixture was stirred at 20° C. for 1 hr. The reaction mixture was quenched with saturated NaHCO₃ (10 mL) and extracted with EtOAc (10 mL×3), dried over Na₂SO₄, filtered and the filtrate was concentrated in vacuum to afford the title compound (30 mg, crude) as a yellow oil. MS (ES⁺) C₂₇H₃₆N₆O₂ requires: 476, found: 477 [M+H]⁺.

### Step 2: (3S,Z)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacyclotridecaphan-8-en-1²-one

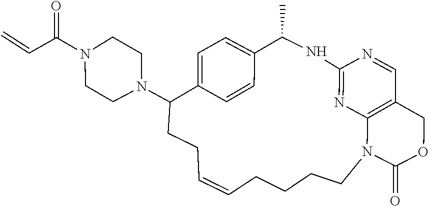 [SMILES: C=CC(=O)N1CCN(C2CC/C=C\CCCCN3C(=O)OCc4cnc(nc43)N[C@@H](C)c3ccc2cc3)CC1]

To a solution of prop-2-enoyl chloride (6.3 mg, 69.2 mol, 5.63 μL, 1.1 eq) in DCM (0.5 mL) was added a solution of (3S,Z)-3-methyl-5-(piperazin-1-yl)-1¹,1⁴-dihydro-1²H-2-aza-1(7,1)-pyrimido[4,5-d][1,3]oxazina-4(1,4)-benzenacyclotridecaphan-8-en-1²-one (30 mg, crude) in DCM (1 mL) dropwise at −78° C., after 2 min at −78° C. a few drops of MeOH was added followed by saturated NaHCO₃ (2 mL), the mixture was allowed to warm to 20° C. To the mixture was added water (8 mL) and extracted with EtOAc (3×8 mL), dried over Na₂SO₄, filtered and concentrated under reduced pressure to give a residue which was purified by prep-HPLC (column: Phenomenex luna C18 150*25 mm*10 μm; mobile phase: [water(FA)-ACN];gradient:8%-38% B over 25 min) and lyophilized to afford the title compound (0.8 mg, 1.3 mol, 2.1% yield, 96% purity, FA salt) as a yellow solid. MS (ES⁺) C₃₀H₃₈N₆O₃ requires: 530, found: 531 [M+H]⁺. ¹H NMR (400 MHz, CD₃OD) δ=8.50-8.39 (m, 1H), 8.05-7.93 (m, 1H), 7.42-7.11 (m, 4H), 6.76-6.62 (m, 1H), 6.23-6.09 (m, 1H), 5.77-5.64 (m, 1H), 5.45-5.20 (m, 2H), 5.18-5.03 (m, 3H), 4.17-3.93 (m, 1H), 3.82-3.44 (m, 5H), 3.24-3.11 (m, 1H), 2.61-2.35 (m, 4H), 2.21-1.26 (m, 9H), 1.19-0.70 (m, 4H).

### Example 3

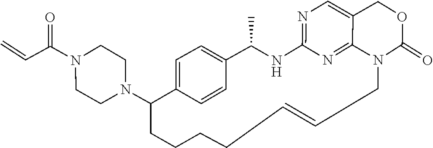 [SMILES: C=CC(=O)N1CCN(C2CCCC/C=C/CN3C(=O)OCc4cnc(nc43)N[C@@H](C)c3ccc2cc3)CC1]

### (3S,E)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacyclododecaphan-10-en-1²-one

### Step 1: (S)—N-(1-(4-bromophenyl)ethyl)-2,2,2-trifluoroacetamide

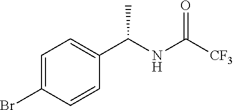 [SMILES: C[C@H](NC(=O)C(F)(F)F)c1ccc(Br)cc1]

To a solution of (S)-1-(4-bromophenyl)ethan-1-amine (11 g, 55.0 mmol) in MeCN (100 mL) at 0° C. was added trifluoroacetic anhydride (8.54 mL, 60.5 mmol, 1.2 eq) and TEA (15.3 mL, 110 mmol, 2.0 eq) dropwise and the resulting mixture was stirred at 0° C. for 0.5 h. Water (200 mL) and brine (100 mL) were added and suspension was stirred for 10 min. The reaction mixture was filtered through Buchner funnel and washed with water and hexanes to give (S)—N-(1-(4-bromophenyl)ethyl)-2,2,2-trifluoroacetamide (15.5 g, 52.3 mmol, 95% yield) as a white solid.

MS (ES⁺) C₁₀H₉BrF₃NO requires: 295, found: 296 [M+H]⁺.

### Step 2: N-methoxy-N-methylhept-6-enamide

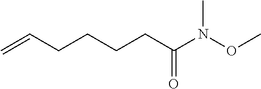

To a solution of hept-6-enoic acid (1 g, 7.8 mmol) in DCM (30 mL) was added CDI (1.3 g, 7.8 mmol, 1.0 eq) and mixture was stirred at 20° C. for 10 min. N,O-dimethylhydroxylamine hydrochloride (0.761 g, 7.80 mmol, 1.0 eq) and DIEA (4.1 mL, 23 mmol, 2.9 eq) was added and the resulting mixture was stirred at 20° C. for 1 h. H₂O (50 mL) was added and the layers were separated. The aqueous phase was extracted with CH₂Cl₂ (2×25 mL), the combined organic layers were washed with sat NaCl, dried over MgSO₄, filtered, and concentrated under reduced pressure. The residue was purified via silica gel chromatography (0-50% EtOAc in hexanes) to give N-methoxy-N-methylhept-6-enamide (1.02 g, 5.97 mmol, 77% yield) as a colorless liquid. MS (ES⁺) C₉H₁₇NO₂ requires: 171, found: 172 [M+H]⁺.

### Step 3: (S)-2,2,2-trifluoro-N-(1-(4-(hept-6-enoyl)phenyl)ethyl)acetamide

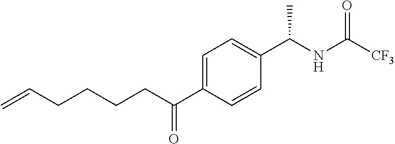 [SMILES: C=CCCCCC(=O)c1ccc([C@H](C)NC(=O)C(F)(F)F)cc1]

To a solution of (S)—N-(1-(4-bromophenyl)ethyl)-2,2,2-trifluoroacetamide (888 mg, 3.00 mmol) in THF (10 mL) at −78° C. was added n-butyllithium (2.64 mL, 6.60 mmol, 2.2 eq) and the resulting mixture was stirred at −78° C. for 0.5 h. N-methoxy-N-methylhept-6-enamide (514 mg, 3 mmol, 1.0 eq) in THF (2 mL) was added dropwise and the mixture was stirred for another 30 min. Sat NH₄Cl (25.0 mL) was added and the layers were separated. The aqueous phase was extracted with EtOAc (3×15 mL), the combined organic layers were washed with sat NaCl, dried over MgSO₄, filtered, and concentrated under reduced pressure. The residue was purified via silica gel chromatography (0-40% EtOAc in hexanes) to give (S)-2,2,2-trifluoro-N-(1-(4-(hept-6-enoyl)phenyl)ethyl)acetamide (442 mg, 1.35 mmol, 45.0% yield) as a white crystalline solid. MS (ES⁺) C₁₇H₂₀F₃NO₂ requires: 327, found: 328 [M+H]⁺.

### Step 4: (S)-1-(4-(1-aminoethyl)phenyl)hept-6-en-1-one

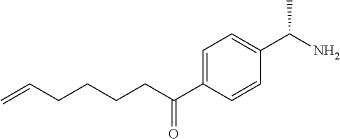 [SMILES: C=CCCCCC(=O)c1ccc([C@H](C)N)cc1]

To a solution of (S)-2,2,2-trifluoro-N-(1-(4-(hept-6-enoyl)phenyl)ethyl)acetamide (440 mg, 1.34 mmol) in EtOH (10 mL) was added KOH (5 M, 1.34 mL, 6.72 mmol, 5.0 eq) and the resulting mixture was stirred at 20° C. for 6 h. The volatiles were removed under reduced pressure. Sat. NaHCO₃ (50 mL) was added and the layers were separated. The aqueous phase was extracted with CH₂Cl₂ (3×20 mL), the combined organic layers were washed with sat NaCl, dried over MgSO₄, filtered, and concentrated under reduced pressure to give (S)-1-(4-(1-aminoethyl)phenyl)hept-6-en-1-one (286 mg, 1.24 mmol, 92% yield) as a colorless liquid. MS (ES⁺) C₁₅H₂₁NO requires: 231, found: 232 [M+H]⁺.

### Step 5: (S)-1-allyl-7-((1-(4-(hept-6-enoyl)phenyl)ethyl)amino)-1,4-dihydro-2H-pyrimido[4,5-d][1,3]oxazin-2-one

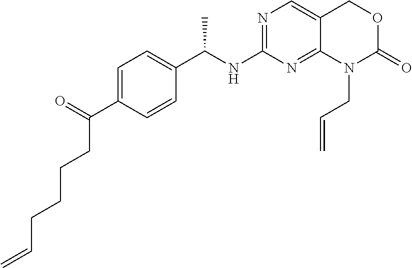 [SMILES: C=CCCCCC(=O)c1ccc([C@H](C)Nc2ncc3c(n2)N(CC=C)C(=O)OC3)cc1]

To a solution of 1-allyl-7-(methylsulfonyl)-1,4-dihydro-2H-pyrimido[4,5-d][1,3]oxazin-2-one (215 mg, 0.798 mmol) in DMSO (3 mL) was added (S)-1-(4-(1-aminoethyl)phenyl)hept-6-en-1-one (185 mg, 0.798 mmol, 1.0 eq), CsF (303 mg, 1.996 mmol, 2.0 eq) and DIEA (0.418 mL, 2.395 mmol, 3.0 eq) and the resulting mixture was stirred at 60° C. for 2 h. H₂O (20 mL) was added and the layers were separated. The aqueous phase was extracted with EtOAc (3×10 mL), the combined organic layers were washed with sat NaCl, dried over MgSO₄, filtered, and concentrated under reduced pressure. The residue was purified via silica gel chromatography (0-100% EtOAc in hexanes) to give (S)-1-allyl-7-((1-(4-(hept-6-enoyl)phenyl)ethyl)amino)-1,4-dihydro-2H-pyrimido[4,5-d][1,3]oxazin-2-one (230 mg, 0.547 mmol, 68.5% yield) as a pale yellow liquid. MS (ES⁺) C₂₄H₂₈N₄O₃ requires: 420, found: 421 [M+H]⁺.

### Step 6: (S,E)-3-methyl-1¹,1⁴-dihydro-1²H-2-aza-1(7,1)-pyrimido[4,5-d][1,3]oxazina-4(1,4)-benzenacyclododecaphan-10-ene-1²,5-dione

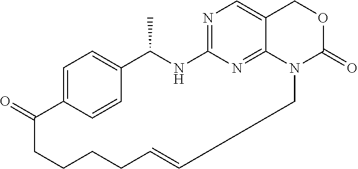 [SMILES: C[C@@H]1Nc2ncc3c(n2)N(C/C=C/CCCCC(=O)c2ccc1cc2)C(=O)OC3]

A solution of (S)-1-allyl-7-((1-(4-(hept-6-enoyl)phenyl)ethyl)amino)-1,4-dihydro-2H-pyrimido[4,5-d][1,3]oxazin-2-one (220 mg, 0.523 mmol) in DCM (200 mL) was degassed with N₂ for 15 minutes followed by the addition of Grubbs II (44.4 mg, 0.052 mmol, 0.1 eq) and the resulting mixture was degassed for an additional 5 minutes then heated to 45° C. and stirred for 48 h. The reaction was cooled to room temperature followed by the addition of ethyl vinyl ether (10.1 mL, 105 mmol) and the volatiles were removed under reduced pressure. The residue was purified via silica gel chromatography (20-100% EtOAc in hexanes) to give (S,E)-3-methyl-1¹,1⁴-dihydro-1²H-2-aza-1(7,1)-pyrimido[4,5-d][1,3]oxazina-4(1,4)-benzenacyclododecaphan-10-ene-1²,5-dione (75 mg, 0.19 mmol, 36.5% yield) as a tan solid. MS (ES⁺) C₂₂H₂₄N₄O₃ requires: 392, found: 393 [M+H]⁺.

### Step 7: Tert-butyl 4-((3S,E)-3-methyl-1²-oxo-1¹,1⁴-dihydro-1²H-2-aza-1(7,1)-pyrimido[4,5-d][1,3]oxazina-4(1,4)-benzenacyclododecaphan-10-en-5-yl)piperazine-1-carboxylate

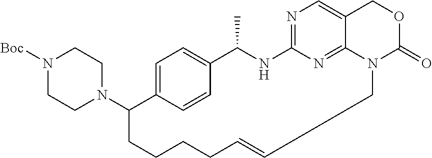 [SMILES: C[C@@H]1Nc2ncc3c(n2)N(C/C=C/CCCCC(N2CCN(C(=O)OC(C)(C)C)CC2)c2ccc1cc2)C(=O)OC3]

To a suspension of (S,E)-3-methyl-1¹,1⁴-dihydro-1²H-2-aza-1(7,1)-pyrimido[4,5-d][1,3]oxazina-4(1,4)-benzenacyclododecaphan-10-ene-1²,5-dione (75 mg, 0.19 mmol) in THF (1 mL) was added tert-butyl piperazine-1-carboxylate (85 mg, 0.46 mmol, 2.4 eq) and titanium(IV) isopropoxide (0.28 mL, 0.96 mmol, 5.1 eq) and the resulting mixture was stirred at 60° C. for 16 h. MeOH (1 mL) was added followed by sodium cyanoborohydride (24 mg, 0.38 mmol, 2.0 eq) and the mixture was stirred at 20° C. for 3 h. The volatiles were removed under reduced pressure. The residue was purified by mass-triggered preparative HPLC (Mobile phase: A=0.1% TFA/H₂O, B=0.1% TFA/MeCN; Gradient: B=40-80%; 12 min; Column: XBridge C18, 5 μm, 19 mm×150 mm) to give a TFA salt. It was dissolved in MeOH and passed through an HCO₃— resin cartridge to give tert-butyl 4-((3S,E)-3-methyl-1²-oxo-1¹,1⁴-dihydro-1²H-2-aza-1(7,1)-pyrimido[4,5-d][1,3]oxazina-4(1,4)-benzenacyclododecaphan-10-en-5-yl)piperazine-1-carboxylate (66 mg, 0.12 mmol, 61% yield) as a colorless liquid. MS (ES⁺) C₃₁H₄₂N₆O₄ requires: 562, found: 563 [M+H]⁺.

### Step 8: (3S,E)-3-methyl-5-(piperazin-1-yl)-1¹,1⁴-dihydro-1²H-2-aza-1(7,1)-pyrimido[4,5-d][1,3]oxazina-4(1,4)-benzenacyclododecaphan-10-en-1²-one 2,2,2-trifluoroacetate

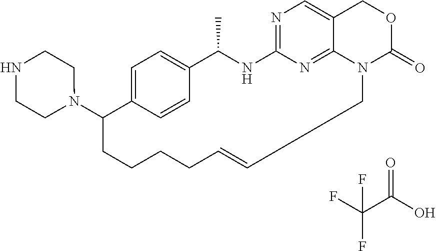 [SMILES: C[C@@H]1Nc2ncc3c(n2)N(C/C=C/CCCCC(N2CCNCC2)c2ccc1cc2)C(=O)OC3 ; O=C(O)C(F)(F)F]

To a solution of tert-butyl 4-((3S,E)-3-methyl-1²-oxo-1¹,1⁴-dihydro-1²H-2-aza-1(7,1)-pyrimido[4,5-d][1,3]oxazina-4(1,4)-benzenacyclododecaphan-10-en-5-yl)piperazine-1-carboxylate (15 mg, 0.027 mmol) in TFA (1 mL) and DCM (1.0 mL) was stirred at 20° C. for 1 h. The volatiles were removed under reduced pressure to give (3S,E)-3-methyl-5-(piperazin-1-yl)-11,14-dihydro-12H-2-aza-1(7,1)-pyrimido[4,5-d][1,3]oxazina-4(1,4)-benzenacyclododecaphan-10-en-12-one 2,2,2-trifluoroacetate (15 mg, 0.026 mmol, 98% yield) as a colorless liquid. MS (ES⁺) C₂₆H₃₄N₆O₂ requires: 462, found: 463 [M+H]⁺.

### Step 9: (3S,E)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacyclododecaphan-10-en-1²-one

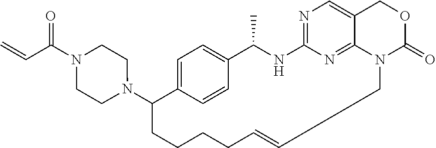 [SMILES: C=CC(=O)N1CCN(C2CCCC/C=C/CN3C(=O)OCc4cnc(nc43)N[C@@H](C)c3ccc2cc3)CC1]

To a solution of (3S,E)-3-methyl-5-(piperazin-1-yl)-1¹,1⁴-dihydro-1²H-2-aza-1(7,1)-pyrimido[4,5-d][1,3]oxazina-4(1,4)-benzenacyclododecaphan-10-en-1²-one 2,2,2-trifluoroacetate (15 mg, 0.026 mmol) in DCM (1 mL) at 0° C. was added DIEA (4.54 μl, 0.026 mmol, 1.0 eq) and acryloyl chloride (2.2 μL, 0.026 mmol, 1.0 eq) and the resulting mixture was stirred at 0° C. for 0.5 h. The volatiles were removed under reduced pressure. The residue was purified by mass-triggered preparative HPLC (Mobile phase: A=0.1% TFA/H₂O, B=0.1% TFA/MeCN; Gradient: B=10-50%; 12 min; Column: XBridge C18, 5 μm, 19 mm×150 mm) to give a TFA salt. It was dissolved in MeOH and passed through an HCO₃-resin cartridge to give (3S,E)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacyclododecaphan-10-en-1²-one (8 mg, 0.02 mmol, 60% yield) as a white solid. MS (ES⁺) C₂₉H₃₆N₆O₃ requires: 516, found: 517 [M+H]⁺. ¹H NMR (600 MHz, MeOD) δ 8.03-7.95 (m, 1H), 7.61-7.05 (m, 4H), 6.75-6.68 (m, 1H), 6.21 (dd, J=16.8, 1.7 Hz, 1H), 5.76 (dd, J=10.6, 1.7 Hz, 1H), 5.19-5.15 (m, 3H), 4.97-4.73 (m, 2H), 4.08-4.00 (m, 1H), 3.85-3.65 (m, 4H), 3.09-2.76 (m, 4H), 2.14-1.68 (m, 2H), 1.55-1.51 (m, 3H), 1.38-0.76 (m, 6H).

### Example 4

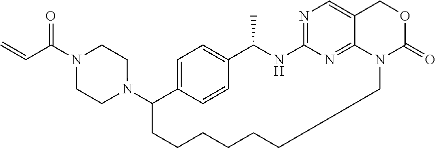 [SMILES: C=CC(=O)N1CCN(C2CCCCCCCN3C(=O)OCc4cnc(nc43)N[C@@H](C)c3ccc2cc3)CC1]

### (3S)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacyclododecaphan-1²-one

### Step 1: Tert-butyl 4-((3S)-3-methyl-12-oxo-1¹,1⁴-dihydro-1²H-2-aza-1(7,1)-pyrimido[4,5-d][1,3]oxazina-4(1,4)-benzenacyclododecaphane-5-yl)piperazine-1-carboxylate

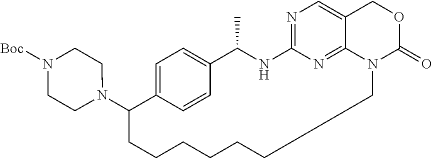 [SMILES: C[C@@H]1Nc2ncc3c(n2)N(CCCCCCCC(N2CCN(C(=O)OC(C)(C)C)CC2)c2ccc1cc2)C(=O)OC3]

A reaction vessel was charged with tert-butyl 4-((3S,E)-3-methyl-1²-oxo-1¹,1⁴-dihydro-1²H-2-aza-1(7,1)-pyrimido[4,5-d][1,3]oxazina-4(1,4)-benzenacyclododecaphan-10-en-5-yl)piperazine-1-carboxylate (50 mg, 0.089 mmol), Pd/C (25 mg, 0.235 mmol) and EtOH (10 mL) under an atmosphere of N₂. The suspension was degassed with N₂ for 3 minutes and purged with H₂ for 3 minutes. The reaction mixture was stirred under an atmosphere of H₂ at 1 atm for 16 h. The reaction mixture was purged with N₂ and filtered through Celite and concentrated under reduced pressure to give tert-butyl 4-((3S)-3-methyl-1²-oxo-1¹,1⁴-dihydro-1²H-2-aza-1(7,1)-pyrimido[4,5-d][1,3]oxazina-4(1,4)-benzenacyclododecaphane-5-yl)piperazine-1-carboxylate (40 mg, 0.071 mmol, 80% yield) as a white solid. MS (ES⁺) C₃₁H₄₄N₆O₄ requires: 564, found: 565 [M+H]⁺.

### Step 2: (3S)-3-methyl-5-(piperazin-1-yl)-1¹,1⁴-dihydro-1²H-2-aza-1(7,1)-pyrimido[4,5-d][1,3]oxazina-4(1,4)-benzenacyclododecaphan-1²-one 2,2,2-trifluoroacetate

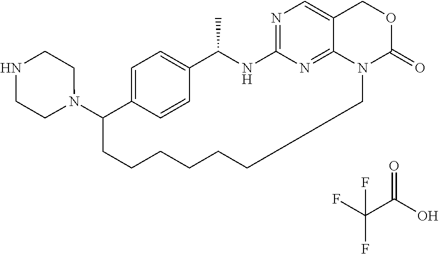 [SMILES: C[C@@H]1Nc2ncc3c(n2)N(CCCCCCCC(N2CCNCC2)c2ccc1cc2)C(=O)OC3 ; O=C(O)C(F)(F)F]

To a solution of tert-butyl 4-((3S)-3-methyl-1²-oxo-1¹, 1⁴-dihydro-1²H-2-aza-1(7,1)-pyrimido[4,5-d][1,3]oxazina-4(1,4)-benzenacyclododecaphane-5-yl)piperazine-1-carboxylate (35 mg, 0.062 mmol) in TFA (1.0 mL) and DCM (1.0 mL) was stirred at 20° C. for 1 h. The volatiles were removed under reduced pressure to give (3S)-3-methyl-5-(piperazin-1-yl)-1¹,1⁴-dihydro-1²H-2-aza-1(7,1)-pyrimido[4,5-d][1,3]oxazina-4(1,4)-benzenacyclododecaphan-1²-one 2,2,2-trifluoroacetate (35 mg, 0.060 mmol, 98% yield) as a colorless liquid. MS (ES⁺) C₂₆H₃₆N₆O₂ requires: 464, found: 465 [M+H]⁺.

### Step 3: (3S)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacyclododecaphan-1²-one

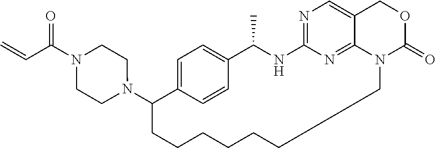 [SMILES: C=CC(=O)N1CCN(C2CCCCCCCN3C(=O)OCc4cnc(nc43)N[C@@H](C)c3ccc2cc3)CC1]

To a solution of (3S)-3-methyl-5-(piperazin-1-yl)-1¹,1⁴-dihydro-1²H-2-aza-1(7,1)-pyrimido[4,5-d][1,3]oxazina-4(1,4)-benzenacyclododecaphan-1²-one 2,2,2-trifluoroacetate (35 mg, 0.060 mmol) in DCM (1 mL) at 0° C. was added DIEA (10.6 μL, 0.060 mmol, 1.0 eq) and acryloyl chloride (4.91 μL, 0.060 mmol, 1.0 eq) and the resulting mixture was stirred at 0° C. for 0.5 h. The volatiles were removed under reduced pressure. The residue was purified by mass-triggered preparative HPLC (Mobile phase: A=0.1% TFA/H₂O, B=0.1% TFA/MeCN; Gradient: B=10-50%; 12 min; Column: XBridge C18, 5 μm, 19 mm×150 mm) to give a TFA salt. It was dissolved in MeOH and passed through an HCO₃-resin cartridge to give (3S)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacyclododecaphan-1²-one (19 mg, 0.037 mmol, 61% yield) as a white solid. MS (ES⁺) C₂₉H₃₈N₆O₃ requires: 518, found: 519 [M+H]⁺. ¹H NMR (600 MHz, MeOD) δ 8.03-7.95 (m, 1H), 7.55-7.11 (m, 4H), 6.76-6.67 (m, 1H), 6.22-6.17 (m, 1H), 5.76-5.72 (m, 1H), 5.16-4.90 (m, 2H), 4.28-3.92 (m, 1H), 3.81-3.48 (m, 6H), 2.97-2.59 (m, 4H), 2.09-1.70 (m, 2H), 1.56-1.50 (m, 3H), 1.39-1.03 (m, 8H), 0.84-0.52 (m, 3H).

### Example 5

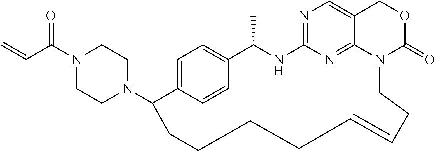 [SMILES: C=CC(=O)N1CCN(C2CCCC/C=C/CCN3C(=O)OCc4cnc(nc43)N[C@@H](C)c3ccc2cc3)CC1]

### (3S,E)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacyclotridecaphan-10-en-12-one

Example 5 was made similarly to Example 3. C₃₀H₃₈N₆O₃ requires: 530.3, found: 531.3 [M+H]⁺. ¹H NMR (600 MHz, METHANOL-d4) δ ppm 8.07-7.99 (m, 1H). 7.57-7.51 (m, 1H), 7.51-7.41 (m, 2H) 7.42-7.31 (m, 1H) 6.77-6.64 (m, 1H) 6.30-6.20 (m, 1H) 5.85-5.72 (m, 1H) 5.42-5.23 (m, 1H) 5.19-4.89 (m, 4H) 4.72-4.59 (m, 1H) 4.34-4.14 (m, 2H) 4.35-4.13 (m, 2H) 3.99-3.89 (m, 1H) 3.85-3.71 (m, 1H) 3.70-3.47 (m, 2H) 2.29-1.64 (m, 6H) 1.63-1.43 (m, 4H) 1.42-0.94 (m, 4H) 0.75-0.62 (m, 1H)

### Example 6

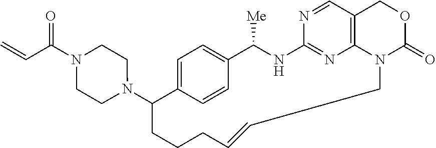 [SMILES: C=CC(=O)N1CCN(C2CCC/C=C/CN3C(=O)OCc4cnc(nc43)N[C@@H](C)c3ccc2cc3)CC1]

### (3S,Z)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacycloundecaphan-9-en-1²-one

### Step 1: 2,2,2-Trifluoro-N-((1S)-1-(4-(1-hydroxyhex-5-en-1-yl)phenyl)ethyl)acetamide

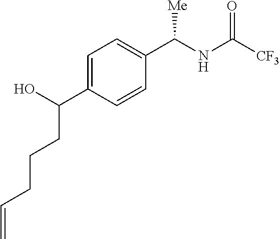 [SMILES: C=CCCCC(O)c1ccc([C@H](C)NC(=O)C(F)(F)F)cc1]

To a cooled −78° C. solution of (S)—N-(1-(4-bromophenyl)ethyl)-2,2,2-trifluoroacetamide (855 mg, 2.89 mmol) in THF (24 mL) was added n-butyllithium (3.0 mL, 6.3 mmol, 2.2 eq). The resulting mixture was stirred at −78° C. for 45 min. A solution of hex-5-enal (350 mg, 3.57 mmol, 1.2 eq) in THF (3.2 mL) was added dropwise at −78° C. and the resulting mixture was stirred at −78° C. and slowly warmed to rt over 16 h. Aqueous ammonium chloride was then added, and the mixture was stirred at rt for 5 min. H₂O (10 mL) was added and the layers were separated. The organic layer was dried over Na₂SO₄, filtered, and concentrated under reduced pressure. The residue was purified via silica gel chromatography (0-15% EtOAc in hexanes) to give 2,2,2-trifluoro-N-((1S)-1-(4-(1-hydroxyhex-5-en-1-yl)phenyl)ethyl)acetamide (441 mg, 1.40 mmol, 48% yield) as a yellow oil. MS (ES⁺) C₁₆H₂₀F₃NO₂ requires: 315, found: 314 [M−H]⁻.

### Step 2: 1-(4-((S)-1-aminoethyl)phenyl)hex-5-en-1-ol

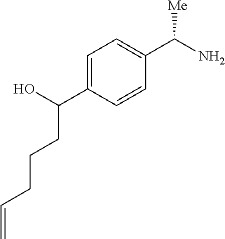 [SMILES: C=CCCCC(O)c1ccc([C@H](C)N)cc1]

To a solution of 2,2,2-trifluoro-N-((1S)-1-(4-(1-hydroxyhex-5-en-1-yl)phenyl)ethyl)acetamide (200 mg, 0.63 mmol) in EtOH (3.2 mL) was added potassium hydroxide (5 M, 620 μL, 3.2 mmol, 5.0 eq) and the resulting mixture was stirred at rt for 3 h. The volatiles were removed under reduced pressure. The residue was resuspended in sat NaHCO₃ (5 mL) and the aqueous phase was extracted with CH₂Cl₂ (3×5 mL), the combined organic layers were washed with sat NaCl, dried over Na₂SO₄, filtered, and concentrated under reduced pressure to yield 1-(4-((S)-1-aminoethyl)phenyl)hex-5-en-1-ol (136 mg, 0.62 mmol, 97% yield) as a yellow oil. MS (ES⁺) C₁₄H₂₁NO requires: 219, found: 203 [M+H—OH]⁺.

### Step 3: 1-allyl-7-(((1S)-1-(4-(1-hydroxyhex-5-en-1-yl)phenyl)ethyl)amino)-1,4-dihydro-2H-pyrimido[4,5-d][1,3]oxazin-2-one

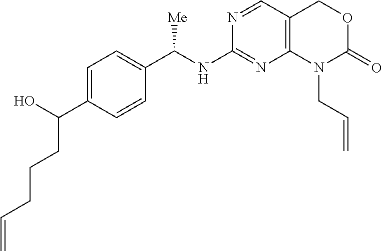 [SMILES: C=CCCCC(O)c1ccc([C@H](C)Nc2ncc3c(n2)N(CC=C)C(=O)OC3)cc1]

To a solution of 1-(4-((S)-1-aminoethyl)phenyl)hex-5-en-1-ol (136 mg, 0.62 mmol) in DMSO (3.0 mL) was added 1-allyl-7-(methylsulfonyl)-1,4-dihydro-2H-pyrimido[4,5-d][1,3]oxazin-2-one (162 mg, 0.60 mmol, 0.97 eq), cesium fluoride (191 mg, 1.3 mmol, 2.0 eq) and DIPEA (350 μl, 2.0 mmol, 3.2 eq) and the resulting mixture was stirred at 60° C. for 16 h. Additional sulfone was added, and the reaction mixture was stirred for an additional 3 h at 60° C. The reaction mixture was diluted with EtOAc (5 mL) and washed with H₂O (2×5 mL). The layers were separated and the organic layer was washed with sat NaCl (5 mL), dried over Na₂SO₄, filtered, and concentrated under reduced pressure to yield 1-allyl-7-(((1S)-1-(4-(1-hydroxyhex-5-en-1-yl)phenyl)ethyl)amino)-1,4-dihydro-2H-pyrimido[4,5-d][1,3]oxazin-2-one (255 mg, 0.62 mmol, 101% yield) as a yellow oil. MS (ES⁺) C₂₃H₂₈N₄O₃ requires: 408, found: 409 [M+H]⁺.

### Step 4: (S)-1-allyl-7-((1-(4-(hex-5-enoyl)phenyl)ethyl)amino)-1,4-dihydro-2H-pyrimido[4,5-d][1,3]oxazin-2-one

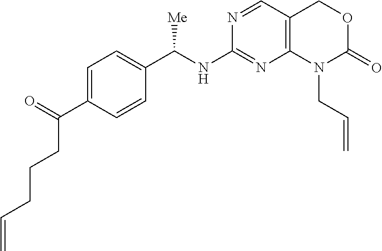 [SMILES: C=CCCCC(=O)c1ccc([C@H](C)Nc2ncc3c(n2)N(CC=C)C(=O)OC3)cc1]

To a solution of 1-allyl-7-(((1S)-1-(4-(1-hydroxyhex-5-en-1-yl)phenyl)ethyl)amino)-1,4-dihydro-2H-pyrimido[4,5-d][1,3]oxazin-2-one (255 mg, 0.56 mmol) in DCM (56 mL) at 0° C. was added DMP (406 mg, 0.96 mmol, 1.7 eq) and the resulting mixture was stirred at rt for 1 h. A 1:1 mixture of sat. NaHCO₃ and 1 M sodium sulfite (50 mL) was added, and the mixture was vigorously stirred for 45 min. The layers were separated and the organic layer was washed with sat NaCl (50 mL), dried over Na₂SO₄, filtered, and concentrated under reduced pressure. The residue was purified via silica gel chromatography (0-100% EtOAc in hexanes) to give (S)-1-allyl-7-((1-(4-(hex-5-enoyl)phenyl)ethyl)amino)-1,4-dihydro-2H-pyrimido[4,5-d][1,3]oxazin-2-one (83 mg, 0.204 mmol, 36% yield) as a pale yellow oil. MS (ES⁺) C₂₃H₂₆N₄O₃ requires: 406, found: 407 [M+H]⁺.

### Step 5: (S)-3-methyl-1¹,1⁴-dihydro-1²H-2-aza-1(7,1)-pyrimido[4,5-d][1,3]oxazina-4(1,4)-benzenacycloundecaphan-9-ene-1²,5-dione

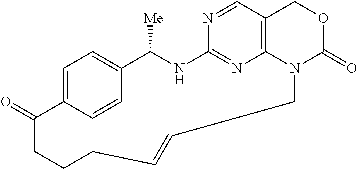 [SMILES: C[C@@H]1Nc2ncc3c(n2)N(C/C=C/CCCC(=O)c2ccc1cc2)C(=O)OC3]

A solution of (S)-1-allyl-7-((1-(4-(hex-5-enoyl)phenyl)ethyl)amino)-1,4-dihydro-2H-pyrimido[4,5-d][1,3]oxazin-2-one (83 mg, 0.204 mmol) in DCM (100 mL) was degassed with N₂ for 15 minutes. Grubbs II (38 mg, 0.045 mmol) was added and the mixture was degassed with N₂ for an additional 5 minutes. The reaction mixture was heated to 40° C. and stirred for 64 h. Additional Grubbs II (41 mg, 0.048 mmol, 0.22 eq) was added, and the reaction was stirred at 40° C. for 20 h. The reaction was cooled to room temperature followed by the addition of ethyl vinyl ether (1.5 mL, 15.6 mmol), and the reaction mixture was stirred at rt for 1 h. The reaction mixture was filtered through silica gel and the filtrate was concentrated under reduced pressure. The residue was purified via silica gel chromatography (0-50, 50-70, then 70-100% EtOAc in hexanes) to give (S)-3-methyl-1¹,1⁴-dihydro-1²H-2-aza-1(7,1)-pyrimido[4,5-d][1,3]oxazina-4(1,4)-benzenacycloundecaphan-9-ene-1²,5-dione (40 mg, 0.11 mmol, 52% yield) as a brown oil. Isolated as a mixture of E and Z isomers. MS (ES⁺) C₂₁H₂₂N₄O₃ requires: 378, found: 379 [M+H]⁺.

### Step 6: Tert-butyl 4-((3S,Z)-3-methyl-1²-oxo-1¹,1⁴-dihydro-1²H-2-aza-1(7,1)-pyrimido[4,5-d][1,3]oxazina-4(1,4)-benzenacycloundecaphan-9-en-5-yl)piperazine-1-carboxylate

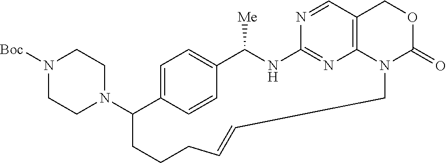 [SMILES: C[C@@H]1Nc2ncc3c(n2)N(C/C=C/CCCC(N2CCN(C(=O)OC(C)(C)C)CC2)c2ccc1cc2)C(=O)OC3]

To a suspension of (S)-3-methyl-1¹,1⁴-dihydro-1²H-2-aza-1(7,1)-pyrimido[4,5-d][1,3]oxazina-4(1,4)-benzenacycloundecaphan-9-ene-1²,5-dione (40 mg, 0.11 mmol) in THF (0.6 mL) was added 1-Boc-piperazine (40 mg, 0.21 mmol, 2.0 eq) and tetraethoxytitanium (120 μl, 0.57 mmol, 5.0 eq) and the resulting mixture was stirred at 60° C. for 16 h. MeOH (0.6 mL) was added followed by sodium cyanoborohydride (17 mg, 0.27 mmol, 2.5 eq) and the mixture was stirred at rt for 3 h. The volatiles were removed under reduced pressure. The residue was purified by mass-triggered preparative HPLC (Mobile phase: A=0.1% TFA/H₂O, B=0.1% TFA/MeCN; Gradient: B=30-70%; 12 min; Column: XBridge C18, 5 μm, 19 mm×150 mm). The collected fractions were combined, concentrated, redissolved in methanol, and passed through a sodium bicarbonate resin. The volatiles were removed under reduced pressure, redissolved in acetonitrile and water and lyophilized to give tert-butyl 4-((3S,Z)-3-methyl-1²-oxo-1¹,1⁴-dihydro-1²H-2-aza-1(7,1)-pyrimido[4,5-d][1,3]oxazina-4(1,4)-benzenacycloundecaphan-9-en-5-yl)piperazine-1-carboxylate (7 mg, 0.013 mmol, 12% yield) as a colorless oil. Isolated as a mixture of E and Z isomers. MS (ES⁺) C₃₀H₄₀N₆O₄ requires: 548, found: 549 [M+H]⁺.

### Step 7: (3S,Z)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacycloundecaphan-9-en-1²-one

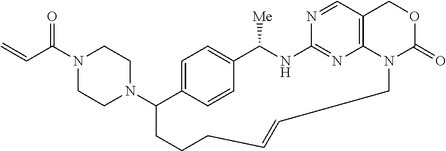 [SMILES: C=CC(=O)N1CCN(C2CCC/C=C/CN3C(=O)OCc4cnc(nc43)N[C@@H](C)c3ccc2cc3)CC1]

To a solution of tert-butyl 4-((3S,Z)-3-methyl-1²-oxo-1¹, 1⁴-dihydro-1²H-2-aza-1(7,1)-pyrimido[4,5-d][1,3]oxazina-4(1,4)-benzenacycloundecaphan-9-en-5-yl)piperazine-1-carboxylate (7 mg, 0.013 mmol) in DCM (0.1 mL) was added TFA (0.1 mL) and the resulting mixture was stirred at rt for 30 min. The volatiles were removed under reduced pressure and the residue was taken forward into the next step without purification. MS (ES⁺) C₂₅H₃₂N₆O₂ requires: 448, found: 449 [M+H]⁺.

To a solution of (3S,Z)-3-methyl-5-(piperazin-1-yl)-1¹,1⁴-dihydro-1²H-2-aza-1(7,1)-pyrimido[4,5-d][1,3]oxazina-4(1,4)-benzenacycloundecaphan-9-en-1²-one bis(2,2,2-trifluoroacetate) (8.8 mg, 0.013 mmol) in DCM (0.1 mL) at −78° C. was added DIPEA (20 μl, 0.115 mmol, 8.8 eq) and acryloyl chloride (0.2 M, 75 μl, 0.015 mmol, 1.2 eq) and the resulting mixture was stirred at −78° C. for 30 min. The reaction mixture was diluted with a few drops of MeOH, sat NaHCO₃ (0.5 mL) was added, the reaction mixture was allowed to warm to rt, and layers were separated. The aqueous phase was extracted with CH₂Cl₂ (2×0.5 mL), the combined organic layers were dried over Na₂SO₄, filtered, and concentrated under reduced pressure. The residue was purified by mass-triggered preparative HPLC (Mobile phase: A=0.1% TFA/H₂O, B=0.1% TFA/MeCN; Gradient: B=20-60%; 12 min; Column: XBridge C18, 5 μm, 19 mm×150 mm), concentrated, then redissolved in MeCN/water and lyophilized to give (3S,Z)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacycloundecaphan-9-en-1²-one (3.7 mg, 7.4 mol, 57% yield) as a sticky, white solid. MS (ES⁺) C₂₈H₃₄N₆O₃ requires: 502, found: 503 [M+H]⁺. ¹H NMR (600 MHz, DMSO) δ 8.05 (s, 1H), 7.97-7.93 (m, 2H), 7.54-7.48 (m, 2H), 6.76 (br s, 1H), 6.15 (dd, J=16.7, 2.2 Hz, 1H), 5.75 (dd, J=10.4, 2.2 Hz, 1H), 5.15-5.04 (m, 3H), 4.57-4.40 (m, 4H), 4.33-4.10 (m, 4H), 3.16-3.06 (m, 2H), 2.14-2.03 (m, 1H), 2.00-1.91 (m, 1H), 1.82-1.73 (m, 1H), 1.71-1.62 (m, 1H), 1.55-1.43 (m, 5H), 1.30-1.22 (m, 4H).

### Example 7 & Example 8

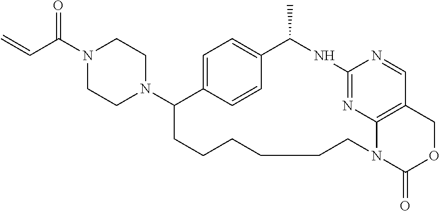 [SMILES: C=CC(=O)N1CCN(C2CCCCCCN3C(=O)OCc4cnc(nc43)N[C@@H](C)c3ccc2cc3)CC1]

### (3S)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacycloundecaphan-1²-one

### Step 1: Tert-butyl 4-[1-[4-[(1S)-1-[(1-allyl-2-oxo-4H-pyrimido[4,5-d][1,3]oxazin-7-yl)amino]ethyl]phenyl]hex-5-enyl]piperazine-1-carboxylate

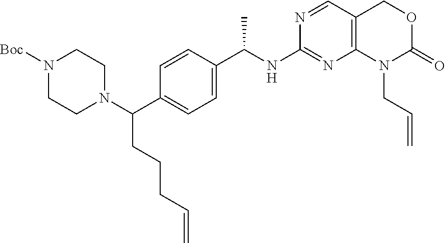 [SMILES: C=CCCCC(c1ccc([C@H](C)Nc2ncc3c(n2)N(CC=C)C(=O)OC3)cc1)N1CCN(C(=O)OC(C)(C)C)CC1]

To a solution of tert-butyl 4-[1-[4-[(1S)-1-aminoethyl]phenyl]hex-5-enyl]piperazine-1-carboxylate (1.15 g, 2.97 mmol, 1 eq) and 1-allyl-7-methylsulfonyl-4H-pyrimido[4,5-d][1,3]oxazin-2-one (0.8 g, 2.97 mmol, 1 eq) in DMF (10 mL) was added CsF (1.35 g, 8.91 mmol, 329 μL, 3 eq) and DIPEA (776 μL, 4.46 mmol, 1.5 eq), the mixture was stirred at 60° C. for 2 h. LCMS showed starting material was consumed completely. The reaction mixture was diluted with H₂O (50 mL) and extracted with EtOAc (3×15 mL). The combined organic phase was washed with brine (15 mL), dried over Na₂SO₄, filtered and concentrated under reduced pressure to give a residue which was purified by flash silica gel chromatography (20 g SepaFlash® Silica Flash Column, Eluent of 50-60% Ethyl acetate/Petroleum ether gradient @100 mL/min) to afford tert-butyl 4-[1-[4-[(1S)-1-[(1-allyl-2-oxo-4H-pyrimido[4,5-d][1,3]oxazin-7-yl)amino]ethyl]phenyl]hex-5-enyl]piperazine-1-carboxylate (0.97 g, 1.6 mmol, 53% yield, 93% purity) as a yellow solid.

MS (ES⁺) C₃₂H₄₄N₆O₄ requires: 576, found 577 [M+H]⁺

### Step 2: Tert-butyl 4-((3S,Z)-3-methyl-1²-oxo-1¹,1⁴-dihydro-1²H-2-aza-1(7,1)-pyrimido[4,5-d][1,3]oxazina-4(1,4)-benzenacycloundecaphan-9-en-5-yl)piperazine-1-carboxylate

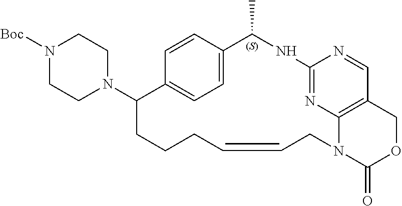

To a solution of tert-butyl 4-[1-[4-[(1S)-1-[(1-allyl-2-oxo-4H-pyrimido[4,5-d][1,3]oxazin-7-yl)amino]ethyl]phenyl]hex-5-enyl]piperazine-1-carboxylate (0.6 g, 1.04 mmol, 1 eq) in DCE (120 mL) was added Hoveyda-Grubbs catalyst 2nd generation (65.2 mg, 104 mol, 0.1 eq) under N₂, the mixture was stirred at 60° C. for 9 h, the mixture was degassed under vacuum and purged with N₂ every 0.5 hour. LCMS showed starting material still remained. Hoveyda-Grubbs catalyst 2^{nd} generation (65.2 mg, 104 mol, 0.1 eq) was added under N₂, and the mixture was stirred at 60° C. for another 6 h. The reaction mixture was concentrated under reduced pressure to give a residue which was purified by flash silica gel chromatography (20 g SepaFlash® Silica Flash Column, Eluent of 50-100% Ethyl acetate/Petroleum ether gradient @100 mL/min). The residue was purified by prep-HPLC (column: Phenomenex Luna C18 150*25 mm*10 μm; mobile phase: [water (FA)-ACN];gradient:20%-38% B over 9 min) to afford the title compound (120 mg, 219 mol, 21.0% yield, 100% purity) as a brown solid.

MS (ES⁺) C₃₀H₄₀N₆O₄ requires: 548, found 549 [M+H]⁺

### Step 3: Tert-butyl 4-((3S)-3-methyl-1²-oxo-1¹,1⁴-dihydro-1²H-2-aza-1(7,1)-pyrimido[4,5-d][1,3]oxazina-4(1,4)-benzenacycloundecaphane-5-yl)piperazine-1-carboxylate

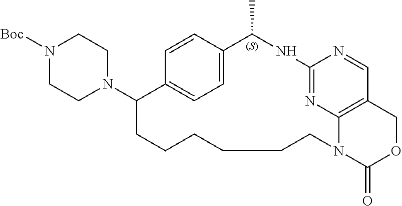

To a mixture of Pd/C (17.5 mg, 16.4 mol, 10% purity, 0.1 eq) in CF₃CH₂OH (9 mL) was added tert-butyl 4-((3S,Z)-3-methyl-1²-oxo-1¹,1⁴-dihydro-1²H-2-aza-1(7,1)-pyrimido[4,5-d][1,3]oxazina-4(1,4)-benzenacycloundecaphan-9-en-5-yl)piperazine-1-carboxylate (90 mg, 164.03 mol, 1 eq) under N₂, the suspension was degassed under vacuum and purged with H₂ several times, the mixture was stirred at 25° C. for 2 h under H₂ (15 psi). LCMS showed starting material was consumed completely. The reaction mixture was filtered and concentrated under reduced pressure to afford the title compound (130 mg, 203 mol, 100% yield, 86% purity) as a yellow solid.

MS (ES⁺) C₃₀H₄₂N₆O₄ requires: 550, found 551 [M+H]⁺

### Step 4: (3S)-3-methyl-5-(piperazin-1-yl)-1¹,1⁴-dihydro-1²H-2-aza-1(7,1)-pyrimido[4,5-d][1,3]oxazina-4(1,4)-benzenacycloundecaphan-1²-one

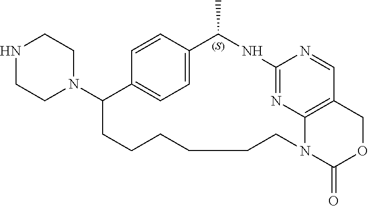

To a solution of tert-butyl 4-((3S)-3-methyl-1²-oxo-1¹,1⁴-dihydro-1²H-2-aza-1(7,1)-pyrimido[4,5-d][1,3]oxazina-4(1,4)-benzenacycloundecaphane-5-yl)piperazine-1-carboxylate (130 mg, 236 mol, 1.0 eq) in dioxane (2 mL) was added HCl/dioxane (4 M, 2 mL, 33.8 eq), the mixture was stirred at 25° C. for 2 h. LCMS showed starting material was consumed completely. The reaction mixture was concentrated under reduced pressure to give a residue which was purified by prep-HPLC (column: Phenomenex Luna C18 150*25 mm*10 μm; mobile phase: [water (TFA)-ACN];gradient:8%-38% B over 10 min) to afford the title compound (61 mg, 108 mol, 37.4% yield, 100% purity, TFA salt) as a yellow solid.

MS (ES⁺) C₂₅H₃₄N₆O₂ requires: 450, found 451 [M+H]⁺

### Step 5: (3S)-5-(4-acryloylpiperazin-1-yl)-3-methyl-1¹,1⁴-dihydro-1²H-2-aza-1(7,1)-pyrimido[4,5-d][1,3]oxazina-4(1,4)-benzenacycloundecaphan-1²-one

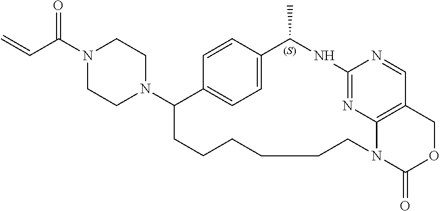

To a mixture of (3S)-3-methyl-5-(piperazin-1-yl)-1¹,1⁴-dihydro-1²H-2-aza-1(7,1)-pyrimido[4,5-d][1,3]oxazina-4(1,4)-benzenacycloundecaphan-1²-one in DCM (1 mL) was added TEA (11.1 μL, 79.7 mol, 1.5 eq) at 20° C., a solution of prop-2-enoyl chloride (4.32 μL, 53.1 μmol, 1.0 eq) in DCM (0.5 mL) was added to the mixture at −78° C. under N₂, the mixture was stirred at −78° C. for 2 min. LCMS showed starting material was consumed completely. The reaction mixture was concentrated under reduced pressure to give a residue which was purified by prep-HPLC (column: Phenomenex Luna C18 150*25 mm*10 μm; mobile phase: [water (TFA)-ACN];gradient:12%-42% B over 10 min), the eluent was freeze dried to afford the title compound (17.1 mg, 93.1 mol, 87.6% yield, 98% purity) as a yellow solid as the first eluting isomer (Example 7).

MS (ES⁺) C₂₈H₃₆N₆O₃ requires: 504, found 505 [M+H]⁺

¹H NMR (400 MHz, CD₃OD) δ=7.97 (s, 1H), 7.48 (dd, J=1.5, 8.1 Hz, 1H), 7.39 (dd, J=1.7, 8.1 Hz, 1H), 7.21 (dd, J=1.7, 7.8 Hz, 1H), 7.06 (dd, J=1.4, 7.8 Hz, 1H), 6.70 (dd, J=10.6, 16.9 Hz, 1H), 6.16 (dd, J=1.9, 16.8 Hz, 1H), 5.71 (dd, J=2.0, 10.6 Hz, 1H), 5.15-5.01 (m, 2H), 4.70 (q, J=6.9 Hz, 1H), 3.82-3.70 (m, 1H), 3.70-3.55 (m, 4H), 3.50-3.38 (m, 1H), 3.17-3.04 (m, 1H), 2.65-2.51 (m, 2H), 2.48-2.34 (m, 2H), 2.17-2.01 (m, 1H), 1.77-1.61 (m, 1H), 1.57-1.38 (m, 4H), 1.25-1.02 (m, 2H), 0.99-0.62 (m, 5H).

Example 8 (11.4 mg, 93.1 mol, 87.6% yield, 100% purity) was afforded as a yellow solid as the second eluting isomer.

MS (ES⁺) C₂₈H₃₆N₆O₃ requires: 504, found 505 [M+H]⁺

¹H NMR (400 MHz, CD₃OD) δ=7.98 (s, 1H), 7.40-7.11 (m, 4H), 6.72 (dd, J=10.6, 16.9 Hz, 1H), 6.18 (dd, J=1.9, 16.8 Hz, 1H), 5.72 (dd, J=2.0, 10.6 Hz, 1H), 5.13-5.02 (m, 2H), 4.75 (q, J=7.1 Hz, 1H), 3.84-3.73 (m, 1H), 3.71-3.56 (m, 4H), 3.50-3.40 (m, 1H), 3.25 (br dd, J=3.7, 9.2 Hz, 1H), 2.66-2.39 (m, 4H), 1.96-1.83 (m, 1H), 1.79-1.64 (m, 1H), 1.54 (d, J=7.0 Hz, 3H), 1.20-1.05 (m, 2H), 1.04-0.93 (m, 2H), 0.91-0.70 (m, 4H).

### Example 9

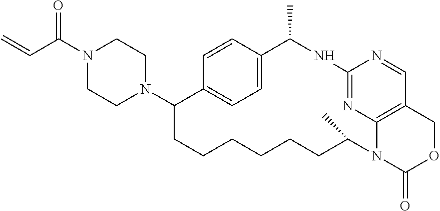 [SMILES: C=CC(=O)N1CCN(C2CCCCCC[C@H](C)N3C(=O)OCc4cnc(nc43)N[C@@H](C)c3ccc2cc3)CC1]

### (3S,12S)-5-(4-acryloylpiperazin-1-yl)-3,12-dimethyl-1¹,1⁴-dihydro-1²H-2-aza-1(7,1)-pyrimido[4,5-d][1,3]oxazina-4(1,4)-benzenacyclododecaphan-1²-one

### Step 1: [(1R)-1-methylbut-3-enyl]4-methylbenzenesulfonate

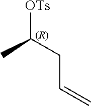

To a solution of (2R)-pent-4-en-2-ol (10 g, 116 mmol, 1 eq), 4-methylbenzenesulfonyl chloride (26.6 g, 139 mmol, 1.2 eq) and DMAP (1.42 g, 11.6 mmol, 0.1 eq) in DCM (200 mL) was added TEA (32.32 mL, 23.5 g, 232.20 mmol, 2 eq) at 0° C. The mixture was stirred at 20° C. for 16 h. TLC (Petroleum ether:Ethyl acetate=3:1) indicated (2R)-pent-4-en-2-ol was consumed completely and two new spots were formed. The reaction mixture was quenched by NH₄Cl (200 mL) and extracted with DCM (2×200 mL), dried over Na₂SO₄, filtered and concentrated under reduced pressure to give a residue which was purified by flash silica gel chromatography (80 g SepaFlash® Silica Flash Column, Eluent of 0-20% Ethyl acetate/Petroleum ether gradient @60 mL/min) to afford [(1R)-1-methylbut-3-enyl]4-methylbenzenesulfonate (25 g, 104 mmol, 89.6% yield) as colorless oil.

¹H NMR (400 MHz, CDCl₃) δ=7.79 (d, J=8.3 Hz, 2H), 7.33 (d, J=7.9 Hz, 2H), 5.68-5.52 (m, 1H), 5.10-4.97 (m, 2H), 4.70-4.60 (m, 1H), 2.44 (s, 3H), 2.40-2.25 (m, 2H), 1.26 (d, J=6.4 Hz, 3H)

### Step 2: (4S)-4-azidopent-1-ene

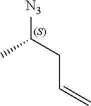

To a solution of [(1R)-1-methylbut-3-enyl]4-methylbenzenesulfonate (23 g, 95.7 mmol, 1.0 eq) in DMF (230 mL) was added NaN₃ (18.5 g, 284 mmol, 3.0 eq) at 20° C. under N₂, the mixture was stirred at 40° C. for 16 h. TLC (Petroleum ether:Ethyl acetate=3:1) indicated [(1R)-1-methylbut-3-enyl]4-methylbenzenesulfonate was consumed completely and two new spots were formed. The reaction mixture was quenched by addition water (200 mL), and then extracted with MTBE (3×200 mL), the combined organic phase was washed with brine (3×300 mL), dried over Na₂SO₄, filtered and concentrated under reduced pressure to afford (4S)-4-azidopent-1-ene (10 g, crude) as colorless oil.

¹H NMR (400 MHz, CDCl₃) δ=5.85-5.75 (m, 1H), 5.20-5.08 (m, 2H), 3.57-3.47 (m, 1H), 2.30-2.19 (m, 2H), 1.26 (d, J=6.5 Hz, 3H)

### Step 3: (2S)-pent-4-en-2-amine

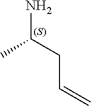

To a solution of (4S)-4-azidopent-1-ene (10 g, 90.0 mmol, 1.0 eq) in THF (100 mL) was added LAH (2.5 M, 72 mL, 180 mmol, 2.0 eq) under protection of N₂ at 0° C., the mixture was stirred at 20° C. for 2 h. TLC (Petroleum ether:Ethyl acetate=3:1) indicated (4S)-4-azidopent-1-ene was consumed completely. The reaction mixture was cooled to 0° C., to the mixture was added water (6.8 mL), NaOH (15%, 6.8 mL), water (30 mL), the mixture was stirred at 20° C. for 10 minutes, filtered and the filter cake was washed with THF (3×20 mL) three times, the filtrate was dried over Na₂SO₄ and filtered to afford (2S)-pent-4-en-2-amine (7 g, crude) as a solution in THF (160 mL).

### Step 4:Ethyl 2-chloro-4-[[(1S)-1-methylbut-3-enyl]amino]pyrimidine-5-carboxylate

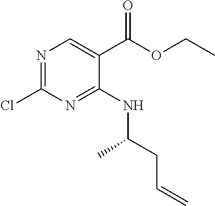 [SMILES: C=CC[C@H](C)Nc1nc(Cl)ncc1C(=O)OCC]

To a solution of (2S)-pent-4-en-2-amine (7 g, 82.2 mmol, 1.0 eq) in THF (160 mL) was added TEA (17.16 mL, 123 mmol, 1.5 eq) and ethyl 2,4-dichloropyrimidine-5-carboxylate (10.9 g, 49.3 mmol, 0.6 eq), the mixture was stirred at 20° C. for 16 h. The reaction mixture was filtered and concentrated under reduced pressure to give a residue which was purified by flash silica gel chromatography (80 g SepaFlash® Silica Flash Column, Eluent of 0-5% Ethyl acetate/Petroleum ether gradient @100 mL/min) to afford ethyl 2-chloro-4-[[(1S)-1-methylbut-3-enyl]amino]pyrimidine-5-carboxylate (4.7 g, 11.3 mmol, 13.7% yield, 65% purity) as yellow oil.

MS (ES⁺) C₁₂H₁₆N₃ClO₂ requires: 269, found: 270 [M+H]⁺,

¹H NMR (400 MHz, CDCl₃) δ=8.65 (s, 1H), 8.37-8.25 (m, 1H), 5.87-5.75 (m, 1H), 5.22-5.06 (m, 2H), 4.44-4.31 (m, 3H), 2.35 (t, J=6.8 Hz, 2H), 1.38 (t, J=7.1 Hz, 3H), 1.26 (d, J=6.6 Hz, 3H).

### Step 5: [2-Chloro-4-[[(1S)-1-methylbut-3-enyl]amino]pyrimidin-5-yl]methanol

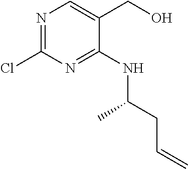

To a solution of ethyl 2-chloro-4-[[(1S)-1-methylbut-3-enyl]amino]pyrimidine-5-carboxylate (1 g, 3.7 mmol, 1.0 eq) in THF (15 mL) was added LAH (2.5 M, 2.22 mL, 5.6 mmol, 1.5 eq) under the protection of N₂ at 0° C., the mixture was stirred at 0° C. for 1 h. LCMS showed 53% of peak with desired mass. To the mixture was added water (0.2 mL), NaOH (15%, 0.2 mL), water (0.5 mL) at 0° C., the mixture was stirred at 20° C. for 10 minutes, filtered and the filter cake was washed with THF (3×20 mL) three times, the filtrate was dried over Na₂SO₄ and filtered was concentrated under reduced pressure to give a residue which was purified by flash silica gel chromatography (12 g SepaFlash® Silica Flash Column, Eluent of 0-30% Ethyl acetate/Petroleum ether gradient @60 mL/min) to afford [2-chloro-4-[[(1S)-1-methylbut-3-enyl]amino]pyrimidin-5-yl]methanol (400 mg, 1.53 mmol, 41.2% yield, 87% purity) as a yellow solid. MS (ES⁺) C₁₀H₁₄N₃C₁₀ requires: 227, found: 228 [M+H]⁺,

### Step 6: [2-Chloro-4-[[(1S)-1-methylbut-3-enyl]amino]pyrimidin-5-yl]methanol

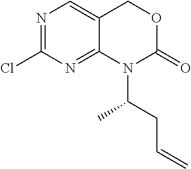 [SMILES: C=CC[C@H](C)N1C(=O)OCc2cnc(Cl)nc21]

To a solution of [2-chloro-4-[[(1S)-1-methylbut-3-enyl]amino]pyrimidin-5-yl]methanol (900. mg, 3.95 mmol, 1.0 eq) in DCM (20 mL) was added DIPEA (2.07 mL, 11.9 mmol, 3 eq) and CDI (961 mg, 5.93 mmol, 1.5 eq), the mixture was stirred at 20° C. for 16 h. The reaction mixture was quenched by water (10 mL) and extracted with DCM (2×25 mL), dried over Na₂SO₄, filtered and concentrated under reduced pressure to give a residue which was purified by flash silica gel chromatography (12 g SepaFlash® Silica Flash Column, Eluent of 10-15% Ethyl acetate/Petroleum ether gradient @100 mL/min) to afford 7-chloro-1-[(1S)-1-methylbut-3-enyl]-4H-pyrimido[4,5-d][1,3]oxazin-2-one (850 mg, 3.25 mmol, 82.2% yield, 97% purity) as yellow oil.

MS (ES⁺) C₁₁H₁₂N₃ClO₂ requires: 253, found: 254 [M+H]⁺,

¹H NMR (400 MHz, CDCl₃) δ=8.22 (s, 1H), 5.83-5.66 (m, 1H), 5.16 (s, 2H), 5.10-4.91 (m, 3H), 2.98-2.87 (m, 1H), 2.63-2.52 (m, 1H), 1.55 (d, J=6.8 Hz, 3H)

### Step 7: N-methoxy-N-methyl-hex-5-enamide

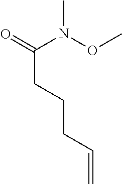

To a suspension of CDI (14.2 g, 87.6 mmol, 1.0 eq) in DCM (50 mL) was added a solution of hex-5-enoic acid (10.4 mL, 87.6 mmol, 1.0 eq) in DCM (100 mL) at 20° C., the resulting mixture was stirred for 15 min at 20° C. and then N-methoxymethanamine hydrochloride (8.55 g, 87.6 mmol, 1.0 eq) was added in portions at 20° C., then TEA (24.4 mL, 175 mmol, 2 eq) was added to the stirring mixture at 20° C., the resulting mixture was stirred at 20° C. for 2 h. TLC (Petroleum ether:Ethyl acetate=1:1) showed consumption of starting material. The reaction mixture was washed with saturated citric acid (3×60 mL) and saturated NaHCO₃ (3×60 mL), dried over Na₂SO₄, filtered and concentrated under reduced pressure to afford N-methoxy-N-methyl-hex-5-enamide (15.9 g, crude) as colorless oil.

¹H NMR (400 MHz, CDCl₃) δ=5.90-5.71 (m, 1H), 5.12-4.90 (m, 2H), 3.75-3.60 (m, 3H), 3.30-3.10 (m, 3H), 2.55-2.35 (m, 2H), 2.25-2.05 (m, 2H), 1.86-1.68 (m, 2H).

### Step 8: 2,2,2-trifluoro-N-[(1S)-1-(4-hex-5-enoylphenyl)ethyl]acetamide

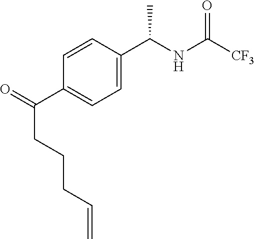 [SMILES: C=CCCCC(=O)c1ccc([C@H](C)NC(=O)C(F)(F)F)cc1]

To a solution of N-[(1S)-1-(4-bromophenyl)ethyl]-2,2,2-trifluoro-acetamide (18.83 g, 63.6 mmol, 1.0 eq) in THF (300 mL) was added n-BuLi (2 M, 63.6 mL, 127 mmol, 2.0 eq) at −78° C. under N₂, the resulting mixture was stirred for 45 min at −78° C. and then a solution of N-methoxy-N-methyl-hex-5-enamide (10 g, 63.3 mmol, 1.0 eq) in THF (30 mL) was added to the stirring mixture at −78° C., the resulting mixture was stirred at −78° C. for 45 min. TLC (Petroleum ether:Ethyl acetate=3:1) indicated N-methoxy-N-methyl-hex-5-enamide was consumed completely. The reaction mixture was quenched by NH₄Cl (300 mL) and extracted with EtOAc (3×300 mL), dried over Na₂SO₄, filtered and concentrated under reduced pressure to give a residue which was purified by flash silica gel chromatography (120 g SepaFlash® Silica Flash Column, Eluent of 10-20% Ethyl acetate/Petroleum ether gradient @200 mL/min) to afford 2,2,2-trifluoro-N-[(1S)-1-(4-hex-5-enoylphenyl)ethyl]acetamide (4.6 g, 14.5 mmol, 22.8% yield, 99% purity) as a white solid.

MS (ES⁺) C₁₆H₁₈O₂NF₃ requires: 313, found: 314 [M+H]⁺,

### Step 9: Tert-butyl 4-[1-[4-[(1S)-1-[(2,2,2-trifluoroacetyl)amino]ethyl]phenyl]hex-5-enyl]piperazine-1-carboxylate

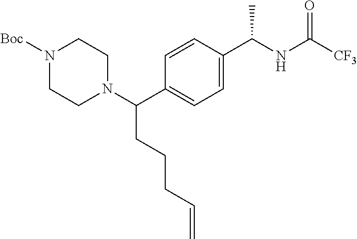 [SMILES: C=CCCCC(c1ccc([C@H](C)NC(=O)C(F)(F)F)cc1)N1CCN(C(=O)OC(C)(C)C)CC1]

To a solution of 2,2,2-trifluoro-N-[(1S)-1-(4-hex-5-enoylphenyl)ethyl]acetamide (800. mg, 2.55 mmol, 1.0 eq) in THF (15 mL) was added Ti(i-PrO)₄ (3.77 mL, 12.8 mmol, 5.0 eq) and tert-butyl piperazine-1-carboxylate (951 mg, 5.11 mmol, 2.0 eq), the mixture was stirred at 60° C. for 16 h, to the mixture was added NaBH₃CN (321 mg, 5.11 mmol, 2.0 eq) and MeOH (15 mL), the mixture was stirred at 20° C. for another 3 h. To the mixture was added saturated NaHCO₃ (30 mL), filtered and filter cake was washed with ethyl acetate (3×50 mL), the filtrate was dried over Na₂SO₄, filtered and concentrated under reduced pressure to give a residue which was purified by flash silica gel chromatography (12 g SepaFlash® Silica Flash Column, Eluent of 15-20% Ethyl acetate/Petroleum ether gradient @60 mL/min) to afford tert-butyl 4-[1-[4-[(1S)-1-[(2,2,2-trifluoroacetyl)amino]ethyl]phenyl]hex-5-enyl]piperazine-1-carboxylate (600 mg, 869 mol, 34.0% yield, 70% purity) as colorless oil.

MS (ES⁺) C₂₅H₃₆N₃O₃F₃ requires: 483, found: 484 [M+H]⁺,

### Step 10: Tert-butyl 4-[1-[4-[(1S)-1-aminoethyl]phenyl]hex-5-enyl]piperazine-1-carboxylate

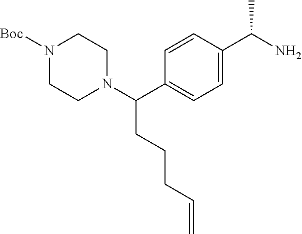 [SMILES: C=CCCCC(c1ccc([C@H](C)N)cc1)N1CCN(C(=O)OC(C)(C)C)CC1]

To a solution of tert-butyl 4-[1-[4-[(1S)-1-[(2,2,2-trifluoroacetyl)amino]ethyl]phenyl]hex-5-enyl]piperazine-1-carboxylate (600 mg, 1.24 mmol, 1.0 eq) in EtOH (8 mL) was added KOH (5 M, 1.24 mL, 6.2 mmol, 5.0 eq) and the resulting mixture was stirred at 20° C. for 16 h. LCMS indicated partial completion. The reaction mixture was quenched by water (10 mL) and extracted with EtOAc (3×30 mL), the combined organic phase was washed with brine (2×60 mL), dried over Na₂SO₄, filtered and concentrated under reduced pressure to afford tert-butyl 4-[1-[4-[(1S)-1-aminoethyl]phenyl]hex-5-enyl]piperazine-1-carboxylate (420 mg, 769 mol, 62.0% yield, 71% purity) as colorless oil.

MS (ES⁺) C₂₃H₃₇N₃O₂ requires: 387, found: 388 [M+H]⁺,

### Step 11: Tert-butyl 4-[1-[4-[(1S)-1-[[1-[(1S)-1-methylbut-3-enyl]-2-oxo-4H-pyrimido[4,5-d][1,3]oxazin-7-yl]amino]ethyl]phenyl]hex-5-enyl]piperazine-1-carboxylate

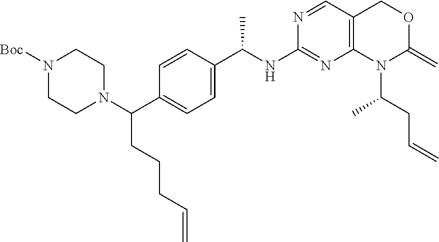 [SMILES: C=CCCCC(c1ccc([C@H](C)Nc2ncc3c(n2)N([C@@H](C)CC=C)C(=C)OC3)cc1)N1CCN(C(=O)OC(C)(C)C)CC1]

To a solution of 7-chloro-1-[(1S)-1-methylbut-3-enyl]-4H-pyrimido[4,5-d][1,3]oxazin-2-one (600 mg, 2.37 mmol, 1.0 eq) and tert-butyl 4-[1-[4-[(1S)-1-aminoethyl]phenyl]hex-5-enyl]piperazine-1-carboxylate (917 mg, 2.37 mmol, 1.0 eq) in DMSO (10 mL) was added DIPEA (618 μL, 3.55 mmol, 1.5 eq), the mixture was stirred at 110° C. for 3 h. The reaction mixture was quenched by water (20 mL) and extracted with EtOAc (50 mL×3), the combined organic phase was washed with brine (2×50 mL), dried over Na₂SO₄, filtered and concentrated under reduced pressure to give a residue which was purified by flash silica gel chromatography (20 g SepaFlash® Silica Flash Column, Eluent of 40-50% Ethyl acetate/Petroleum ether gradient @80 mL/min) to afford tert-butyl 4-[1-[4-[(1S)-1-[[1-[(1S)-1-methylbut-3-enyl]-2-oxo-4H-pyrimido[4,5-d][1,3]oxazin-7-yl]amino]ethyl]phenyl]hex-5-enyl]piperazine-1-carboxylate (540. mg, 759 mol, 32.0% yield, 85% purity) as a yellow solid.

MS (ES⁺) C₃₄H₄₈N₆O₄ requires: 604, found: 605 [M+H]⁺,

### Step 12: Tert-butyl 4-((3S,12S,E)-3,12-dimethyl-1²-oxo-1¹,1⁴-dihydro-1²H-2-aza-1(7,1)-pyrimido[4,5-d][1,3]oxazina-4(1,4)-benzenacyclododecaphan-9-en-5-yl)piperazine-1-carboxylate

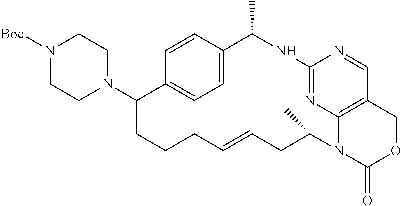 [SMILES: C[C@@H]1Nc2ncc3c(n2)N(C(=O)OC3)[C@@H](C)C/C=C/CCCC(N2CCN(C(=O)OC(C)(C)C)CC2)c2ccc1cc2]

To a solution of tert-butyl 4-[1-[4-[(1S)-1-[[1-[(1S)-1-methylbut-3-enyl]-2-oxo-4H-pyrimido[4,5-d][1,3]oxazin-7-yl]amino]ethyl]phenyl]hex-5-enyl]piperazine-1-carboxylate (250. mg, 413 mol, 1.0 eq) in DCE (25 mL) was added Hoveyda-Grubb's 2nd (51.8 mg, 82.7 mol, 0.2 eq), the mixture was stirred at 60° C. for 9 h under N₂, the mixture was purged with N₂ several times every 0.5 hour. The reaction mixture was concentrated under reduced pressure to give a residue which was purified by prep-HPLC (column: Phenomenex luna C18 150*25 mm*10 μm; mobile phase: [water(FA)-ACN];gradient:18%-48% B over 10 min) and lyophilized to afford the title compound (45 mg, 67.9 mol, 16.4% yield, 87% purity) as a brown solid.

MS (ES⁺) C₃₂H₄₄N₆O₄ requires: 576, found: 577 [M+H]⁺,

### Step 13: Tert-butyl 4-((3S,12S)-3,12-dimethyl-1²-oxo-1¹,1⁴-dihydro-1²H-2-aza-1(7,1)-pyrimido[4,5-d][1,3]oxazina-4(1,4)-benzenacyclododecaphane-5-yl)piperazine-1-carboxylate

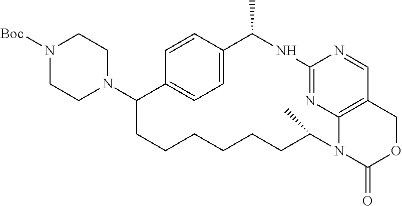 [SMILES: C[C@@H]1Nc2ncc3c(n2)N(C(=O)OC3)[C@@H](C)CCCCCCC(N2CCN(C(=O)OC(C)(C)C)CC2)c2ccc1cc2]

To a solution of Pd/C (24.9 mg, 23.4 mol, 10% purity, 0.3 eq) in CF₃CH₂OH (1 mL) was added tert-butyl 4-((3S,12S,E)-3,12-dimethyl-1²-oxo-1¹,1⁴-dihydro-1²H-2-aza-1(7,1)-pyrimido[4,5-d][1,3]oxazina-4(1,4)-benzenacyclododecaphan-9-en-5-yl)piperazine-1-carboxylate (45 mg, 78. μmol, 1.0 eq) in CF₃CH₂OH (2 mL) under N₂, the suspension was degassed under vacuum and purged with H₂ several times, the mixture was stirred at 25° C. for 2.5 h under H₂ (15 psi). The reaction mixture was filtered and concentrated in vacuum to afford the title compound (45 mg, 66.1 mol, 84.7% yield, 85% purity) as a brown solid.

MS (ES⁺) C₃₂H₄₆N₆O₄ requires: 578, found: 579 [M+H]⁺,

### Step 14: (3S,12S)-3,12-dimethyl-5-(piperazin-1-yl)-1¹,1⁴-dihydro-1²H-2-aza-1(7,1)-pyrimido[4,5-d][1,3]oxazina-4(1,4)-benzenacyclododecaphan-1²-one

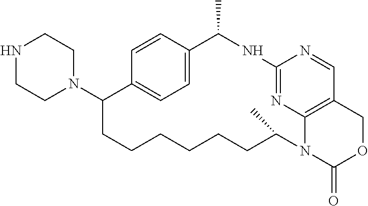 [SMILES: C[C@@H]1Nc2ncc3c(n2)N(C(=O)OC3)[C@@H](C)CCCCCCC(N2CCNCC2)c2ccc1cc2]

To a solution of tert-butyl 4-((3S,12S)-3,12-dimethyl-1²-oxo-1¹,1⁴-dihydro-1²H-2-aza-1(7,1)-pyrimido[4,5-d][1,3]oxazina-4(1,4)-benzenacyclododecaphane-5-yl)piperazine-1-carboxylate (60 mg, 104 mol, 1.0 eq) in dioxane (2 mL) was added HCl/dioxane (4 M, 2 mL, 77.1 eq), the mixture was stirred at 25° C. for 1 h. The reaction mixture was concentrated in vacuum to afford (3S,15S)-3,15-dimethyl-8-piperazin-1-yl-18-oxa-2,16,22,23-tetrazatetracyclo[14.6.2.2^{4.7}.0^{20,24}]hexacosa-1(23),4(26),5,7(25),20(24),21-hexaen-17-one (95 mg, crude, HCl salt) as a brown solid.

MS (ES⁺) C₂₇H₃₈N₆O₂ requires: 478, found: 479 [M+H]⁺,

### Step 15: (3S,12S)-5-(4-acryloylpiperazin-1-yl)-3,12-dimethyl-1¹,1⁴-dihydro-1²H-2-aza-1(7,1)-pyrimido[4,5-d][1,3]oxazina-4(1,4)-benzenacyclododecaphan-1²-one

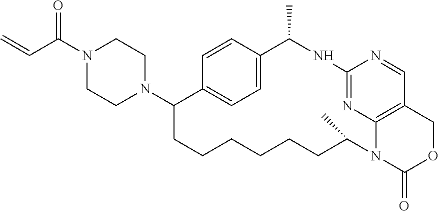 [SMILES: C=CC(=O)N1CCN(C2CCCCCC[C@H](C)N3C(=O)OCc4cnc(nc43)N[C@@H](C)c3ccc2cc3)CC1]

To a solution of prop-2-enoyl chloride (5.91 μL, 72.8 mol, 1 eq) in DCM (1 mL) was added a solution of (3S,12S)-3,12-dimethyl-5-(piperazin-1-yl)-1¹,1⁴-dihydro-1²H-2-aza-1(7,1)-pyrimido[4,5-d][1,3]oxazina-4(1,4)-benzenacyclododecaphan-1²-one (75 mg, 72.8 mol, 1.0 eq, HCl) and TEA (10.13 μL, 72.8 mol, 1 eq) in DCM (1 mL) at −78° C., the mixture was stirred at −78° C. for 2 min. The reaction mixture was concentrated in vacuum to give a residue which was purified by prep-HPLC (column: Phenomenex luna C18 150*25 mm*10 μm; mobile phase: [water(TFA)-ACN];gradient:17%-47% B over 10 min) and lyophilized to afford the title compound (26.1 mg, 40.0 mol, 54.8% yield, 99% purity, TFA) as a white solid.

MS (ES⁺) C₃₀H₄₀N₆O₃ requires: 532, found: 533 [M+H]⁺,

¹H NMR (400 MHz, CD₃OD) δ=8.05 (s, 1H), 7.72-7.27 (m, 4H), 6.76-6.66 (m, 1H), 6.29-6.20 (m, 1H), 5.83-5.76 (m, 1H), 5.14-4.91 (m, 3H), 4.58-4.35 (m, 1H), 4.34-4.15 (m, 1H), 4.00-3.34 (m, 2H), 3.27-2.74 (m, 4H), 2.35-2.10 (m, 2H), 1.67-0.44 (m, 18H)

### Example 10

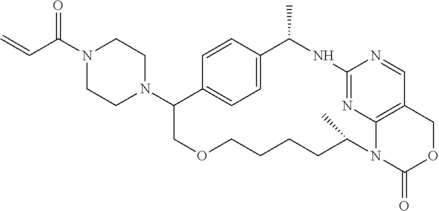 [SMILES: C=CC(=O)N1CCN(C2COCCCC[C@H](C)N3C(=O)OCc4cnc(nc43)N[C@@H](C)c3ccc2cc3)CC1]

### (3S,12S)-5-(4-acryloylpiperazin-1-yl)-3,12-dimethyl-1¹,1⁴-dihydro-1²H-7-oxa-2-aza-1(7,1)-pyrimido[4,5-d][1,3]oxazina-4(1,4)-benzenacyclododecaphan-1²-one

### Step 1: 2-Allyloxyacetic acid

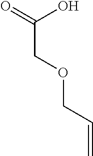

To a mixture of prop-2-en-1-ol (4.95 mL, 72.8 mmol, 1.01 eq) in THF (100 mL) was added NaH (8.64 g, 216 mmol, 60% purity, 3.0 eq) at 0° C. under N₂, and the mixture was stirred at 20° C. for 0.5 h, a solution of 2-bromoacetic acid (5.17 mL, 72.0 mmol, 1.0 eq) in THF (20 mL) was added slowly at 0° C. and the mixture was stirred at 20° C. for 16 h. TLC (Dichloromethane:Methanol=20:1) showed starting material was consumed. The reaction mixture was quenched by saturated NH₄Cl (50 mL), the pH was adjusted to 5 by HCl (1 N) and extracted with EtOAc (3×50 mL), the combined organic phase was dried over Na₂SO₄, filtered and concentrated under reduced pressure to afford 2-allyloxyacetic acid (11 g, crude) as a brown oil.

¹H NMR (400 MHz, DMSO-d₆) δ=12.58 (br s, 1H), 5.95-5.80 (m, 1H), 5.30-5.20 (m, 1H), 5.19-5.11 (m, 1H), 4.02-3.97 (m, 4H).

### Step 2: 2-Allyloxy-N-methoxy-N-methyl-acetamide

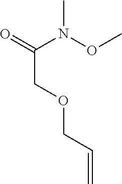

To a suspension of CDI (15.4 g, 94.7 mmol, 1.0 eq) in DCM (100 mL) was added a solution of 2-allyloxyacetic acid (11 g, 94.7 mmol, 1.0 eq) in DCM (30 mL) at 20° C., the resulting mixture was stirred for 15 min at 20° C. and then N-methoxymethanamine (9.24 g, 94.7 mmol, 1.0 eq, HCl salt) was added in portions at 20° C., then TEA (26.4 mL, 189 mmol, 2.0 eq) was added to the stirring mixture at 20° C. The resulting mixture was stirred at 20° C. for 16 h. TLC (Dichloromethane:Methanol=20:1) showed starting material was consumed. The reaction mixture was washed with saturated citric acid (3×100 mL) and saturated NaHCO₃ (3×100 mL), dried over Na₂SO₄, filtered and concentrated under reduced pressure to afford 2-allyloxy-N-methoxy-N-methyl-acetamide (9.6 g, 60.3 mmol, 63.6% yield,) as brown oil.

¹H NMR (400 MHz, CDCl₃) δ=6.01-5.88 (m, 1H), 5.35-5.28 (m, 1H), 5.25-5.20 (m, 1H), 4.27 (s, 2H), 4.14-4.12 (m, 2H), 3.68 (s, 3H), 3.19 (s, 3H).

### Step 3: N-[(1S)-1-[4-(2-allyloxyacetyl)phenyl]ethyl]-2,2,2-trifluoro-acetamide

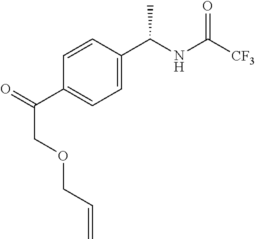 [SMILES: C=CCOCC(=O)c1ccc([C@H](C)NC(=O)C(F)(F)F)cc1]

n-BuLi (2.5 M, 34.5 mL, 86.3 mmol, 2.3 eq) was added dropwise to a solution of N-[(1S)-1-(4-bromophenyl)ethyl]-2,2,2-trifluoro-acetamide (11.16 g, 37.7 mmol, 1.0 eq) in THF (200 mL) at −78° C. under N₂, the mixture was stirred for 45 min at −78° C., then a solution of 2-allyloxy-N-methoxy-N-methyl-acetamide (6 g, 37.7 mmol, 1.0 eq) in THF (20 mL) was added at −78° C. under N₂, the mixture was stirred at −78° C. for 45 min under N₂. LCMS showed starting material was consumed completely. The reaction mixture was quenched by saturated NH₄Cl. (50 mL) and extracted with EtOAc (3×50 mL). The combined organic phase was dried over Na₂SO₄, filtered and concentrated under reduced pressure to give a residue which was purified by flash silica gel chromatography (80 g SepaFlash® Silica Flash Column, Eluent of 0-33% Ethyl acetate/Petroleum ether gradient @150 mL/min) to afford N-[(1S)-1-[4-(2-allyloxyacetyl)phenyl]ethyl]-2,2,2-trifluoro-acetamide (3.7 g, 11.3 mmol, 29.8% yield, 96% purity) as a white solid.

MS (ES⁻) C₁₅H₁₆O₃NF₃ requires: 315, found 314 [M−H]⁻.

### Step 4: Tert-butyl 4-[2-allyloxy-1-[4-[(1S)-1-[(2,2,2-trifluoroacetyl)amino]ethyl]phenyl]ethyl]piperazine-1-carboxylate

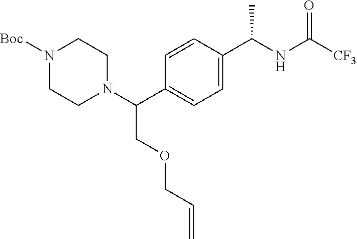 [SMILES: C=CCOCC(c1ccc([C@H](C)NC(=O)C(F)(F)F)cc1)N1CCN(C(=O)OC(C)(C)C)CC1]

To a solution of N-[(1S)-1-[4-(2-allyloxyacetyl)phenyl]ethyl]-2,2,2-trifluoro-acetamide (1 g, 3.17 mmol, 1.0 eq) and tert-butyl piperazine-1-carboxylate (591 mg, 3.17 mmol, 1 eq) in MeOH (20 mL) was added HOAc (182 μL, 3.17 mmol, 1.0 eq) and NaBH₃CN (199 mg, 3.17 mmol, 1.0 eq), the mixture was stirred at 65° C. for 16 h. LCMS showed starting material remained. The reaction mixture was diluted with H₂O (30 mL) and extracted with EtOAc (3×20 mL). The combined organic phase was dried over Na₂SO₄, filtered and concentrated under reduced pressure to give a residue which was purified by flash silica gel chromatography (20 g SepaFlash® Silica Flash Column, Eluent of 10-15% Ethyl acetate/Petroleum ether gradient @150 mL/min) to afford tert-butyl 4-[2-allyloxy-1-[4-[(1S)-1-[(2,2,2-trifluoroacetyl)amino]ethyl]phenyl]ethyl]piperazine-1-carboxylate (1.9 g, 1.84 mmol, 28.9% yield, 47% purity) as yellow oil.

MS (ES⁻) C₂₄H₃₄N₃O₄F₃ requires: 485, found 484 [M−H]⁻.

### Step 5: Tert-butyl 4-[2-allyloxy-1-[4-[(1S)-1-aminoethyl]phenyl]ethyl]piperazine-1-carboxylate

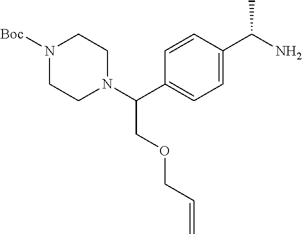 [SMILES: C=CCOCC(c1ccc([C@H](C)N)cc1)N1CCN(C(=O)OC(C)(C)C)CC1]

To a solution of tert-butyl 4-[2-allyloxy-1-[4-[(1S)-1-[(2,2,2-trifluoroacetyl)amino]ethyl]phenyl]ethyl]piperazine-1-carboxylate (2.67 g, 5.50 mmol, 1.0 eq) in dioxane (10 mL) was added LiOH·H₂O (1 M, 16.5 mL, 16.5 mmol, 3.0 eq) at 0° C., the mixture was stirred at 25° C. for 16 h. LCMS showed starting material was consumed completely. The reaction mixture was diluted with H₂O (20 mL) and extracted with EtOAc (3×20 mL). The combined organic phase was dried over Na₂SO₄, filtered and concentrated under reduced pressure to give a residue which was purified by prep-HPLC (column: Phenomenex Luna C18 150*40 mm*15 μm; mobile phase: [water (TFA)-ACN]; gradient: 10%-40% B over 10 min), the pH of eluent was adjusted to 8 by saturated NaHCO₃ and extracted with EtOAc (5×50 mL). The combined organic phase was dried over Na₂SO₄, filtered and concentrated under reduced pressure to afford tert-butyl 4-[2-allyloxy-1-[4-[(1S)-1-aminoethyl]phenyl]ethyl]piperazine-1-carboxylate (1 g, 2.28 mmol, 41.5% yield, 89% purity) as yellow oil.

MS (ES⁺) C₂₂H₃₅N₃O₃ requires: 389, found 390 [M+H]⁺.

### Step 6: tert-butyl 4-[2-allyloxy-1-[4-[(1S)-1-[[1-[(1S)-1-methylbut-3-enyl]-2-oxo-4H-pyrimido[4,5-d][1,3]oxazin-7-yl]amino]ethyl]phenyl]ethyl]piperazine-1-carboxylate

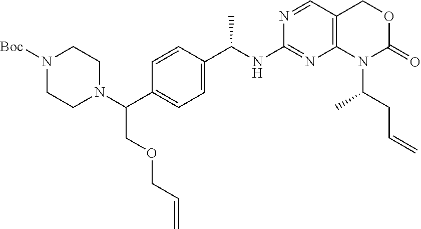 [SMILES: C=CCOCC(c1ccc([C@H](C)Nc2ncc3c(n2)N([C@@H](C)CC=C)C(=O)OC3)cc1)N1CCN(C(=O)OC(C)(C)C)CC1]

To a solution of tert-butyl 4-[2-allyloxy-1-[4-[(1S)-1-aminoethyl]phenyl]ethyl]piperazine-1-carboxylate (811 mg, 2.08 mmol, 1.1 eq) and 7-chloro-1-[(1S)-1-methylbut-3-enyl]-4H-pyrimido[4,5-d][1,3]oxazin-2-one (480 mg, 1.89 mmol, 1.0 eq) in DMSO (10 mL) was added DIPEA (494 μL, 2.84 mmol, 1.5 eq), the resulting mixture was stirred at 110° C. for 1.5 h. LCMS showed starting material was consumed completely. The reaction mixture was diluted with H₂O (40 mL) and extracted with EtOAc (3×10 mL). The combined organic phase was washed with brine (10 mL), dried over Na₂SO₄, filtered and concentrated under reduced pressure to give a residue which was purified by flash silica gel chromatography (20 g SepaFlash® Silica Flash Column, Eluent of 90-100% Ethyl acetate/Petroleum ether gradient @150 mL/min) to afford tert-butyl 4-[2-allyloxy-1-[4-[(1S)-1-[[1-[(1S)-1-methylbut-3-enyl]-2-oxo-4H-pyrimido[4,5-d][1,3]oxazin-7-yl]amino]ethyl]phenyl]ethyl]piperazine-1-carboxylate (500 mg, 783 mol, 41.3% yield, 95% purity) as a yellow solid.

MS (ES⁺) C₃₃H₄₆N₆O₅ requires: 606, found 607 [M+H]⁺.

### Step 7: Tert-butyl 4-((3S,12S,E)-3,12-dimethyl-1²-oxo-1¹,1⁴-dihydro-1²H-7-oxa-2-aza-1(7,1)-pyrimido[4,5-d][1,3]oxazina-4(1,4)-benzenacyclododecaphan-9-en-5-yl)piperazine-1-carboxylate

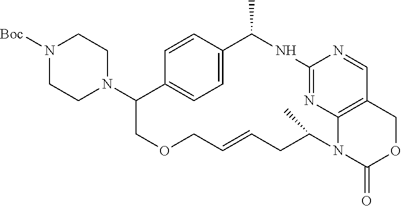 [SMILES: C[C@@H]1Nc2ncc3c(n2)N(C(=O)OC3)[C@@H](C)C/C=C/COCC(N2CCN(C(=O)OC(C)(C)C)CC2)c2ccc1cc2]

To a solution of tert-butyl 4-[2-allyloxy-1-[4-[(1S)-1-[[1-[(1S)-1-methylbut-3-enyl]-2-oxo-4H-pyrimido[4,5-d][1,3]oxazin-7-yl]amino]ethyl]phenyl]ethyl]piperazine-1-carboxylate (450. mg, 742 mol, 1.0 eq) in DCE (90 mL) was added Hoveyda-Grubb's 2^{nd} generation catalyst (46.5 mg, 74.2 mol, 0.1 eq) under N₂, the mixture was stirred at 60° C. for 9 h. LCMS showed that the starting material remained. The reaction mixture was concentrated under reduced pressure to give a residue which was purified by prep-HPLC (column: Phenomenex Luna C18 150*25 mm*10 μm; mobile phase: [water (FA)-ACN]; gradient:20%-50% B over 10 min), the pH of eluent was adjusted to 8 by saturated NaHCO₃ and extracted with EtOAc (3×30 mL). The combined organic phase was dried over Na₂SO₄, filtered and concentrated under reduced pressure to afford the title compound (120 mg, 205 mol, 27.6% yield, 99% purity) as a yellow solid.

MS (ES⁺) C₃₁H₄₂N₆O₅ requires: 578, found 579 [M+H]⁺.

### Step 8: Tert-butyl 4-((3S,12S)-3,12-dimethyl-1²-oxo-1¹,1⁴-dihydro-1²H-7-oxa-2-aza-1(7,1)-pyrimido[4,5-d][1,3]oxazina-4(1,4)-benzenacyclododecaphane-5-yl)piperazine-1-carboxylate

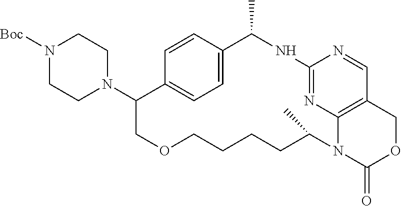 [SMILES: C[C@@H]1Nc2ncc3c(n2)N(C(=O)OC3)[C@@H](C)CCCCOCC(N2CCN(C(=O)OC(C)(C)C)CC2)c2ccc1cc2]

To a mixture of Pd/C (18.4 mg, 17.3 mol, 10% purity, 0.1 eq) in CF₃CH₂OH (5 mL) was added tert-butyl 4-((3S,12S,E)-3,12-dimethyl-1²-oxo-1¹,1⁴-dihydro-1²H-7-oxa-2-aza-1(7,1)-pyrimido[4,5-d][1,3]oxazina-4(1,4)-benzenacyclododecaphan-9-en-5-yl)piperazine-1-carboxylate (100 mg, 173 mol, 1.0 eq) under N₂, the suspension was degassed under vacuum and purged with H₂ several times, the mixture was stirred at 25° C. for 3 h under H₂ (15 psi). The reaction mixture was filtered and concentrated under reduced pressure to afford the title compound (120 mg, crude) as a yellow solid.

MS (ES⁺) C₃₁H₄₄N₆O₅ requires: 580, found 581 [M+H]⁺.

### Step 9: (3S,12S)-3,12-dimethyl-5-(piperazin-1-yl)-1¹,1⁴-dihydro-1²H-7-oxa-2-aza-1(7,1)-pyrimido[4,5-d][1,3]oxazina-4(1,4)-benzenacyclododecaphan-1²-one

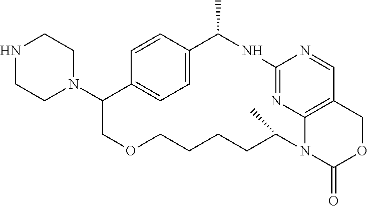 [SMILES: C[C@@H]1Nc2ncc3c(n2)N(C(=O)OC3)[C@@H](C)CCCCOCC(N2CCNCC2)c2ccc1cc2]

To a mixture of tert-butyl 4-((3S,12S)-3,12-dimethyl-1²-oxo-1¹,1⁴-dihydro-1²H-7-oxa-2-aza-1(7,1)-pyrimido[4,5-d][1,3]oxazina-4(1,4)-benzenacyclododecaphane-5-yl)piperazine-1-carboxylate (120. mg, 207 mol, 1.0 eq) in DCM (2 mL) was added TFA (0.5 mL, 6.73 mmol, 32.6 eq), the mixture was stirred at 25° C. for 1 h. LCMS showed starting material was consumed completely. The reaction mixture was added dropwise into saturated NaHCO₃ (10 mL), extracted with EtOAc (3×5 mL). The combined organic phase was dried over Na₂SO₄, filtered and concentrated under reduced pressure to afford the title compound (0.14 g, 198 mol, 95.8% yield, 68% purity) as a yellow solid.

MS (ES⁺) C₂₆H₃₆N₆O₃ requires: 480, found 481 [M+H]⁺.

### Step 10: (3S,12S)-5-(4-acryloylpiperazin-1-yl)-3,12-dimethyl-1¹,1⁴-dihydro-1²H-7-oxa-2-aza-1(7,1)-pyrimido[4,5-d][1,3]oxazina-4(1,4)-benzenacyclododecaphan-1²-one

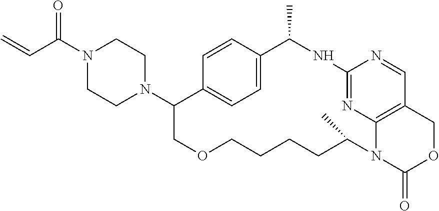 [SMILES: C=CC(=O)N1CCN(C2COCCCC[C@H](C)N3C(=O)OCc4cnc(nc43)N[C@@H](C)c3ccc2cc3)CC1]

To a mixture of (3S,12S)-3,12-dimethyl-5-(piperazin-1-yl)-1¹,1⁴-dihydro-1²H-7-oxa-2-aza-1(7,1)-pyrimido[4,5-d][1,3]oxazina-4(1,4)-benzenacyclododecaphan-1²-one (0.14 g, 291 μmol, 1.0 eq) in DCM (2 mL) was added TEA (60.8 μL, 437 mol, 1.5 eq) at 20° C., a solution of prop-2-enoyl chloride (16.6 μL, 204 mol, 0.7 eq) in DCM (1 mL) was added to the mixture at −78° C. under N₂, the mixture was stirred at −78° C. for 2 min. LCMS showed starting material was consumed completely. The reaction mixture was concentrated under reduced pressure to give a residue which was purified by prep-HPLC (column: Phenomenex Luna C18 150*25 mm*10 μm; mobile phase: [water (FA)-ACN];gradient:12%-32% B over 9 min), the eluent was freeze dried to afford the title compound (20 mg, 32.7 mol, 11.2% yield, 95% purity, FA salt) as a white solid.

MS (ES⁺) C₂₉H₃₈N₆O₄ requires: 534, found 535 [M+H]⁺.

¹H NMR (400 MHz, CD₃OD) δ=8.24 (br s, 1H), 7.98 (s, 1H), 7.56-7.08 (m, 4H), 6.79-6.64 (m, 1H), 6.24-6.10 (m, 1H), 5.76-5.67 (m, 1H), 5.01 (s, 2H), 4.42-4.30 (m, 1H), 3.96-3.76 (m, 1H), 3.75-3.54 (m, 5H), 3.51-3.34 (m, 2H), 3.28-2.97 (m, 2H), 2.59-2.40 (m, 4H), 1.64-0.72 (m, 12H).

The activity of the compounds in Examples 1-10 as mutant IDH inhibitors is illustrated in the following assays. The compounds described herein can be tested for efficacy in the treatment or prevention of symptoms or indications of mutant IDH-mediated diseases using techniques well known to those in the art.

### Biological Activity Assays

mIDH1 R132H/S280F

The enzymatic activity of mIDH1_{R132H/S280F} was assessed by measuring the fluorescence of the NADPH. The assay was performed in 384 well, white ProxiPlates (PerkinElmer, 6008280) in assay buffer containing 50 mM Hepes (Life Technologies, 15630080), 50 mM KCl (Teknova, P0327), 10 mM MgCl₂ (Sigma-Aldrich, M1028), 1 mM DTT (Sigma-Aldrich, 43815), 0.02% BSA (Sigma-Aldrich, A3059) at pH 7.5. The final reaction volume was 10 μL. Stock solutions of the test compounds were prepared in 100% DMSO (Sigma, D2650) and serially diluted 1:3 using 100% DMSO. 50 nL of compound were then transferred to the assay plate using the Beckman Coulter ECHO 650 Acoustic Liquid Handler for a final DMSO concentration of 0.5%. 5 μL of a mIDH1_{R132H/S280F} and NADPH (Santa Cruz, SC202725C) mixture were added to the assay plate for a final concentration of 2 nM and 10 PM, respectively. 5 μL of NADPH alone were added to negative control wells followed by a 1 hour preincubation at room temperature. 5 μL of alphaketoglutarate (Sigma-Aldrich, K1128) were added to the assay plate for a final concentration of 120 μM. Fluorescence of NADPH (excitation: 350 nm, emission 450 nm) was immediately measured kinetically for approximately 90 minutes using the BioTek Synergy Neo plate reader.

mIDH1 R132H

The enzymatic activity of mIDH1_{R132H} was assessed by measuring the fluorescence of the NADPH. The assay was performed in 384 well, white ProxiPlates (PerkinElmer, 6008280) in assay buffer containing 50 mM Hepes (Life Technologies, 15630080), 50 mM KCl (Teknova, P0327), 10 mM MgCl₂ (Sigma-Aldrich, M1028), 1 mM DTT (Sigma-Aldrich, 43815), 0.02% BSA (Sigma-Aldrich, A3059) at pH 7.5. The final reaction volume was 10 μL. Stock solutions of the test compounds were prepared in 100% DMSO (Sigma, D2650) and serially diluted 1:3 using 100% DMSO. 50 nL of compound were then transferred to the assay plate using the Beckman Coulter ECHO 650 Acoustic Liquid Handler for a final DMSO concentration of 0.5%. L of a mIDH1_{R132H} and NADPH (Santa Cruz, SC202725C) mixture were added to the assay plate for a final concentration of 10 nM and 20 μM, respectively. 5 μL of NADPH alone were added to negative control wells. 5 μL of alphaketoglutarate (Sigma-Aldrich, K1128) were added to the assay plate for a final concentration of 200 μM. Fluorescence of NADPH (excitation: 350 nm, emission 450 nm) was immediately measured kinetically for approximately 60 minutes using the BioTek Synergy Neo plate reader.

mIDH2 R140Q

The enzymatic activity of mIDH2_{R140Q} was assessed by measuring the fluorescence of the NADPH. The assay was performed in 384 well, white ProxiPlates (PerkinElmer, 6008280) in assay buffer containing 50 mM Hepes (Life Technologies, 15630080), 50 mM KCl (Teknova, P0327), 10 mM MgCl₂ (Sigma-Aldrich, M1028), 1 mM DTT (Sigma-Aldrich, 43815), 0.02% BSA (Sigma-Aldrich, A3059) at pH 7.5. The final reaction volume was 10 μL. Stock solutions of the test compounds were prepared in 100% DMSO (Sigma, D2650) and serially diluted 1:3 using 100% DMSO. 50 nL of compound were then transferred to the assay plate using the Beckman Coulter ECHO 650 Acoustic Liquid Handler for a final DMSO concentration of 0.5%. 5 μL of a mIDH2_{R140Q} and NADPH (Santa Cruz, SC202725C) mixture were added to the assay plate for a final concentration of 3 nM and 10 μM, respectively. 5 μL of NADPH alone were added to negative control wells followed by a 1 hour preincubation at room temperature. 5 μL of alphaketoglutarate (Sigma-Aldrich, K1128) were added to the assay plate for a final concentration of 300 μM. Fluorescence of NADPH (excitation: 350 nm, emission 450 nm) was immediately measured kinetically for approximately 150 minutes using the BioTek Synergy Neo plate reader.

### WT IDH1

The enzymatic activity of WT IDH1 was assessed by measuring the fluorescence of the NADPH generated in the enzymatic reaction. The assay was performed in 384 well, white ProxiPlates (PerkinElmer, 6008280) in assay buffer containing 50 mM Hepes (Life Technologies, 15630080), 50 mM KCl (Teknova, P0327), 10 mM MgCl₂ (Sigma-Aldrich, M1028), 1 mM DTT (Sigma-Aldrich, 43815), 0.02% BSA (Sigma-Aldrich, A3059) at pH 7.5. The final reaction volume was 10 μL. Stock solutions of the test compounds were prepared in 100% DMSO (Sigma, D2650) and serially diluted 1:3 using 100% DMSO. 50 nL of compound were then transferred to the assay plate using the Beckman Coulter ECHO 650 Acoustic Liquid Handler for a final DMSO concentration of 0.5%. 5 μL of WT IDH1 were added to the assay plate for a final concentration of 0.5 nM. 5 μL of assay buffer alone were added to negative control wells. 5 μL of an isocitrate (Sigma-Aldrich, 58790) and NADP (Sigma-Aldrich, N₅₇₅₅) mixture were added to the assay plate for a final concentration of 5 μM and 15 μM, respectively. Fluorescence of NADPH (excitation: 350 nm, emission 450 nm) was immediately measured kinetically for approximately 15 minutes using the BioTek Synergy Neo plate reader.

### WT IDH2

The enzymatic activity of WT IDH2 was assessed by measuring the fluorescence of the NADPH generated in the enzymatic reaction. The assay was performed in 384 well, white ProxiPlates (PerkinElmer, 6008280) in assay buffer containing 50 mM Hepes (Life Technologies, 15630080), 50 mM KCl (Teknova, P0327), 10 mM MgCl₂ (Sigma-Aldrich, M1028), 1 mM DTT (Sigma-Aldrich, 43815), 0.02% BSA (Sigma-Aldrich, A3059) at pH 7.5. The final reaction volume was 10 μL. Stock solutions of the test compounds were prepared in 100% DMSO (Sigma, D2650) and serially diluted 1:3 using 100% DMSO. 50 nL of compound were then transferred to the assay plate using the Beckman Coulter ECHO 650 Acoustic Liquid Handler for a final DMSO concentration of 0.5%. 5 μL of WT IDH2 were added to the assay plate for a final concentration of 0.5 nM. 5 μL of assay buffer alone were added to negative control wells. 5 μL of an isocitrate (Sigma-Aldrich, 58790) and NADP (Sigma-Aldrich, N₅₇₅₅) mixture were added to the assay plate for a final concentration of 5 μM and 15 μM, respectively. Fluorescence of NADPH (excitation: 350 nm, emission 450 nm) was immediately measured kinetically for approximately 15 minutes using the BioTek Synergy Neo plate reader.

TABLE 1
IDH Enzyme Assays

| | IDH1_{R132H/S280F} | IDH2_{R140Q} | IDH1_{R132H} | IDH1 WT | IDH2 WT |
|---|---|---|---|---|---|
| Example | IC₅₀ (nM) | IC₅₀ (nM) | IC₅₀ (nM) | IC₅₀ (nM) | IC₅₀ (nM) |
| 1 | 103 | 1323 | n.d. | n.d. | n.d. |
| 2 | 284 | 1835 | n.d. | n.d. | n.d. |
| 3 | 260 | 1438 | 3.3 | 3166 | 16214 |
| 4 | 295 | 1680 | 4 | >16667 | 41215 |
| 5 | n.d. | n.d. | n.d. | n.d. | n.d. |
| 6 | n.d. | n.d. | n.d. | n.d. | n.d. |
| 7 | 295 | 1728 | n.d. | n.d. | n.d. |
| 8 | 922 | 4727 | n.d. | n.d. | n.d. |
| 9 | 94 | 1492 | n.d. | n.d. | n.d. |
| 10 | 83 | 4994 | n.d. | n.d. | n.d. |

### Cell-Based Assays

### U87 IDH1 R132H

The IDH1 mutant-U-87 isogenic cell line (ATCC HTB-14IG) used to test cellular inhibition is a glioma IDH1_{R132H} mutant isogenic line derived from the parental U-87MG cell line. These cells are maintained in EMEM (ATCC 30-2003) culture media with 10% FBS and 10 U/mL Penicillin-Streptomycin. Twelve thousand cells are plated in polystyrene 384 well cell culture plates (Greiner Bio-One 781091) (12,000 cells/well) approximately 18 hours before compound treatment. Immediately before treatment, the cells are washed twice with PBS, followed by culture media replenishing. Cells are then treated with different concentrations of test compounds (5 μM to 0.3 pM) for 24 h. Extracellular 2HG levels are measured from cell culture media. For analyte extraction, 30 μL of culture media is transferred to 120 uL 100% acetonitrile in a deep 384 well plate and mixed well, followed by 5 min centrifugation at 3700 rpm at room temperature. 100 μL of supernatant is transferred to a flat bottom 384 well plate. Samples are run on the Agilent RapidFire-Mass Spectrometry instrument to detect 2HG levels. The samples from each well were aspirated for 600 ms and loaded onto a HILIC solid-phase extraction cartridge (Agilent) followed by a wash step for 3000 ms with 90% acetonitrile containing 5 mM ammonium acetate. Analytes were then eluted off the cartridge into the Agilent 6460 Triple Quad mass spectrometer with a 4500 ms elution step using 30% acetonitrile containing 5 mM ammonium acetate. The HILIC cartridge was then re-equilibrated by 90% acetonitrile containing 5 mM ammonium acetate for 500 ms. The 2HG was detected by an Agilent 6460 Triple Quad mass spectrometer in negative ion mode. Multiple reaction monitoring was used to quantify 2HG using the precursor ion and product ion at m/z 147.03 and 128.9, respectively. The percent activity of each sample is calculated by using DMSO and control compound treated references, and IC₅₀ values are obtained using the GeneData Screener data analysis program.

### TF1 IDH2 R140Q

The IDH2 mutant-TF1 isogenic cell line (ATCC CRL-20031G) used to test cellular inhibition is an erythroblast cell line that was isolated from the marrow of a patient with erythroleukemia with the IDH2_{R140Q} mutation. These cells are maintained in RPMI-1640 (ATCC 30-2001) culture media with 10% FBS, 10 U/mL Penicillin-Streptomycin, and 2 ng/mL GM-CSF. Thirty thousand cells are plated in polystyrene tissue culture treated 384 well cell culture plates (PerkinElmer 6007680) (30,000 cells/well) approximately 2 hours before compound treatment. Cells are then treated with different concentrations of test compounds (5 μM to 0.3 pM) for 24 hr. Extracellular 2HG levels are measured from cell culture media. For analyte extraction, 30 μL of culture media is transferred to 120 μL 100% acetonitrile in a deep 384 well plate and mixed well, followed by 5 min centrifugation at 3700 rpm at room temperature. 100 μL of supernatant is transferred to a flat bottom 384 well plate. Samples are run on the Agilent RapidFire-Mass Spectrometry instrument to detect 2HG levels. The samples from each well were aspirated for 600 ms and loaded onto a HILIC solid-phase extraction cartridge (Agilent) followed by a wash step for 3000 ms with 90% acetonitrile containing 5 mM ammonium acetate. Analytes were then eluted off the cartridge into the Agilent 6460 Triple Quad mass spectrometer with a 4500 ms elution step using 30% acetonitrile containing 5 mM ammonium acetate. The HILIC cartridge was then re-equilibrated by 90% acetonitrile containing 5 mM ammonium acetate for 500 ms. The 2HG was detected by an Agilent 6460 Triple Quad mass spectrometer in negative ion mode. Multiple reaction monitoring was used to quantify 2HG using the precursor ion and product ion at m/z 147.03 and 128.9, respectively. The percent activity of each sample is calculated by using DMSO and control compound treated references, and IC₅₀ values are obtained using the GeneData Screener data analysis program.

TABLE 2
Mutant IDH 2-HG Cellular Assay

| | U87 IDH1_{R132H} | TF1 IDH2_{R140Q} |
|---|---|---|
| Example | 2-HG IC₅₀ (nM) | 2-HG IC₅₀ (nM) |
| 1 | 0.32 | 269 |
| 2 | 0.31 | 342 |
| 3 | 0.14 | 277 |
| 4 | 0.20 | 282 |
| 5 | 0.10 | 215 |
| 6 | 0.26 | 1722 |
| 7 | 0.24 | 123 |
| 8 | 0.19 | 351 |
| 9 | 0.14 | 504 |
| 10 | 0.31 | 1218 |

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this disclosure, and without departing from the spirit and scope thereof, can make various changes and modifications of the disclosure to adapt it to various usages and conditions.

1. A compound of Formula I or Formula II 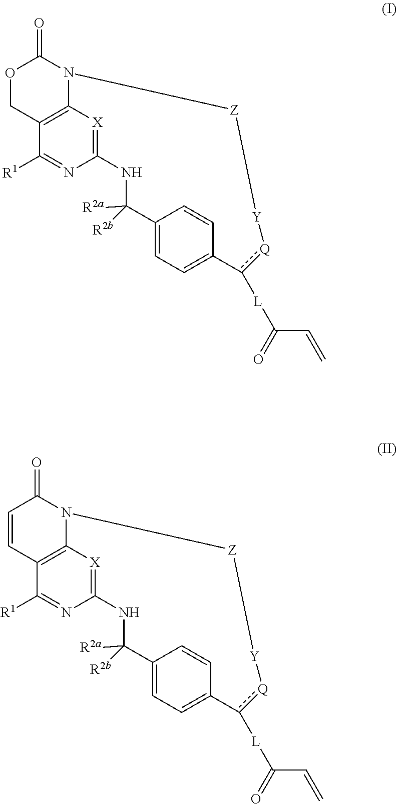
or a pharmaceutically acceptable salt thereof, wherein
L is chosen from C₁₋₆ alkylene, C₃₋₇ cycloalkylene, 3- to 7-membered heterocycloalkylene, arylene, and heteroarylene, any of which may be optionally substituted with one or more R³;
Q is chosen from —O—, —CR^{c}R^{d}—, and ═CR^{c}—;
X is chosen from CH, CF, and N;
Y is absent or is chosen from arylene and heteroarylene, either of which may be optionally substituted with one or more R³;
Z is a saturated or partially unsaturated bivalent C₄₋₁₀ hydrocarbon chain optionally substituted with one or more R^{a}, wherein 1 or 2 nonconsecutive methylene units of the chain may be independently replaced by —O—, —OC(O)—, —C(O)O—, —C(O)—, —NR^{b}C(O)—, —C(O)NR^{b}—, —OC(O)NR^{b}—, or —NR^{b}C(O)O—;
each R^{a} is independently chosen from C₁₋₆ alkyl, C₃₋₈ cycloalkyl, halo, C₁₋₆ haloalkyl, C₁₋₆ alkoxy, aryl, and heteroaryl;
each R^{b} is independently chosen from hydrogen and C₁₋₆ alkyl;
R^{c} is chosen from hydrogen, C₁₋₆ alkyl, C₃₋₈ cycloalkyl, halo, C₁₋₆ haloalkyl, C₁₋₆ alkoxy, aryl, and heteroaryl;
R^{d} is chosen from hydrogen and C₁₋₆ alkyl;
R¹ is chosen from hydrogen, halo, hydroxyl, and amino;
R^{2a} and R^{2b} are independently chosen from hydrogen, methyl, and halomethyl; and
each R³ is independently chosen from C₁₋₆ alkyl, C₃₋₇ cycloalkyl, C₁₋₆haloalkyl, 3- to 7-membered heterocycloalkyl, C₁₋₆ alkoxy, C₁₋₆ haloalkoxy, cyano, halo, and hydroxy.

2. The compound of claim 1, or a pharmaceutically acceptable salt thereof, wherein L is 3- to 7-membered heterocycloalkylene.

3. The compound of claim 2, or a pharmaceutically acceptable salt thereof, wherein L is chosen from piperazinyl and piperidinyl.

4. The compound of claim 3, or a pharmaceutically acceptable salt thereof, wherein L is

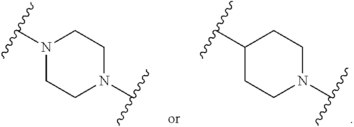 [SMILES: *N1CCN(*)CC1 ; *C1CCN(*)CC1]

5. The compound of claim 1, or a pharmaceutically acceptable salt thereof, wherein X is N.

6. (canceled)

7. (canceled)

8. (canceled)

9. (canceled)

10. The compound of claim 1, having the structure of Formula IA or IIA 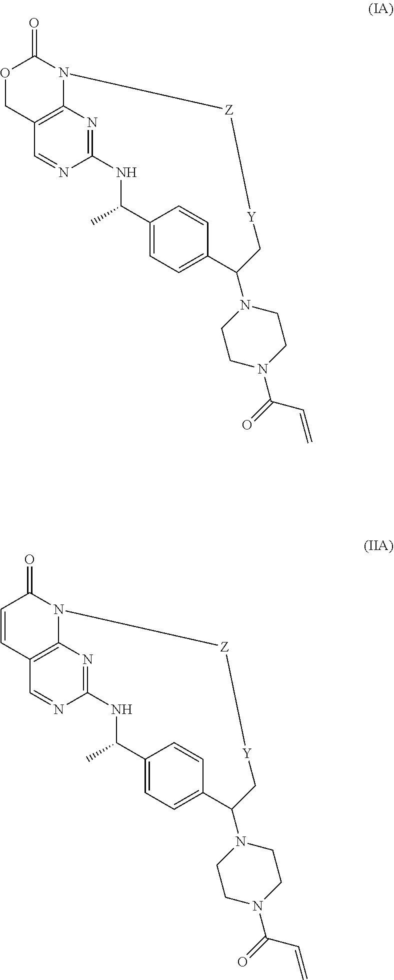
or a pharmaceutically acceptable salt thereof.

11. (canceled)

12. (canceled)

13. (canceled)

14. (canceled)

15. (canceled)

16. The compound of claim 1, having the structure of Formula IB or IIB 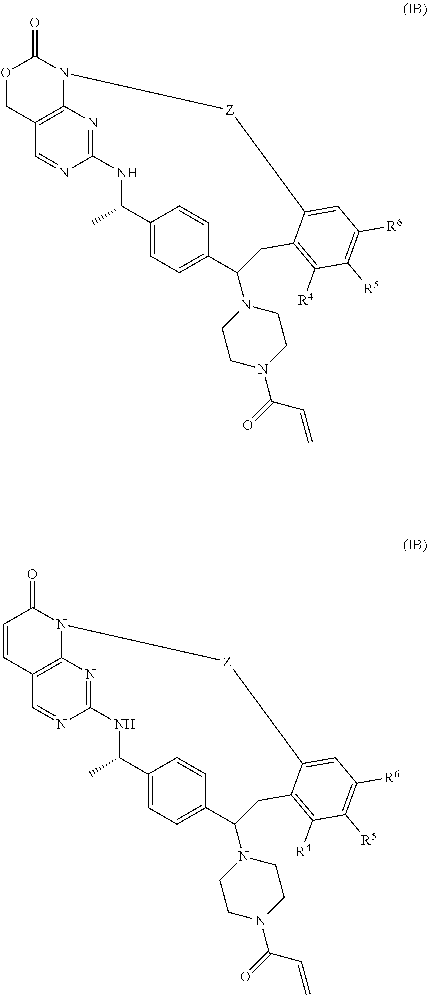
or a pharmaceutically acceptable salt thereof, wherein:
R⁴ is chosen from hydrogen, C₁₋₆ alkyl, C₁₋₆ haloalkyl, C₁₋₆ haloalkoxy, C₃₋₇ cycloalkyl, and halo;
R⁵ is chosen from hydrogen, C₁₋₆ alkyl, C₁₋₆ haloalkyl, C₁₋₆ haloalkoxy, and C₃₋₇ cycloalkyl; and
R⁶ is chosen from hydrogen, C₁₋₆ alkyl, and C₁₋₆ haloalkyl.

17. The compound of claim 16, or a pharmaceutically acceptable salt thereof, wherein R⁴ is chosen from hydrogen, methyl, trifluoromethyl, trifluoromethoxy, cyclopropyl, and fluoro.

18. The compound of claim 16, or a pharmaceutically acceptable salt thereof, wherein R⁵ is chosen from hydrogen, methyl, trifluoromethyl, trifluoromethoxy, and cyclopropyl.

19. The compound of claim 16, or a pharmaceutically acceptable salt thereof, wherein R⁶ is chosen from hydrogen, methyl, and trifluoromethyl.

20. The compound of claim 16, or a pharmaceutically acceptable salt thereof, wherein Z is chosen from —O(CH₂)₄— and —(CH₂)₅—, either of which are optionally substituted with one R^{a}.

21. (canceled)

22. (canceled)

23. The compound of claim 1, or a pharmaceutically acceptable salt thereof, wherein Q is chosen from —O—, and —CR^{c}R^{d}—.

24. (canceled)

25. (canceled)

26. (canceled)

27. (canceled)

28. (canceled)

29. (canceled)

30. The compound of claim 1, having the structure of Formula IC or IIC 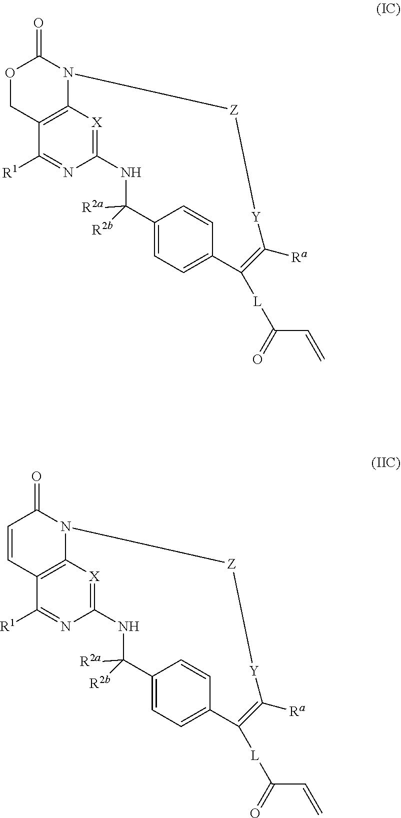
or a pharmaceutically acceptable salt thereof.

31. The compound of claim 1, having a structure chosen from 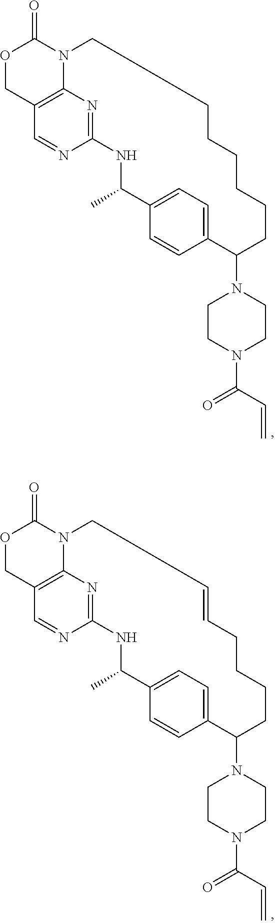 [SMILES: C=CC(=O)N1CCN(C2CCCCCCCN3C(=O)OCc4cnc(nc43)N[C@@H](C)c3ccc2cc3)CC1 ; C=CC(=O)N1CCN(C2CCCC/C=C/CN3C(=O)OCc4cnc(nc43)N[C@@H](C)c3ccc2cc3)CC1] 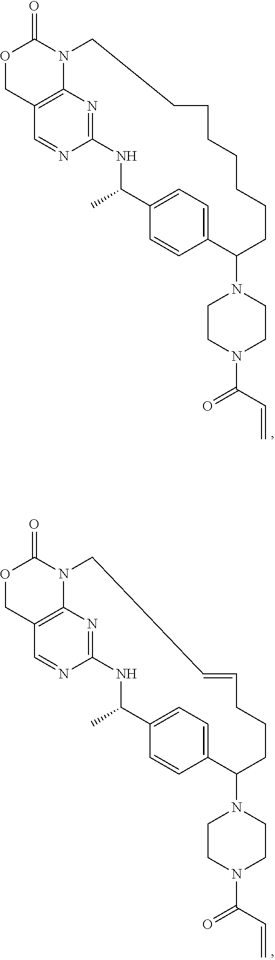 [SMILES: C=CC(=O)N1CCN(C2CCCCCCCCN3C(=O)OCc4cnc(nc43)N[C@@H](C)c3ccc2cc3)CC1 ; C=CC(=O)N1CCN(C2CCC/C=C/CN3C(=O)OCc4cnc(nc43)N[C@@H](C)c3ccc2cc3)CC1] 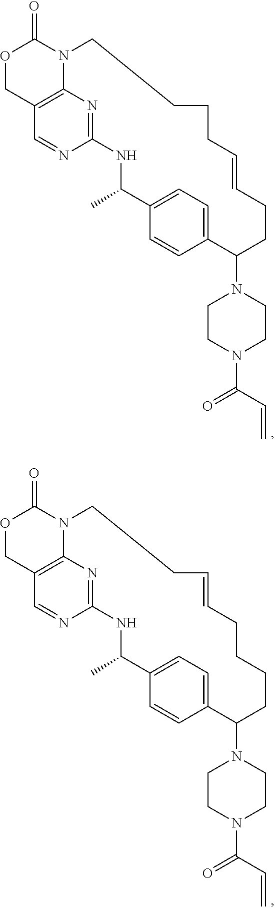 [SMILES: C=CC(=O)N1CCN(C2CC/C=C/CCCCN3C(=O)OCc4cnc(nc43)N[C@@H](C)c3ccc2cc3)CC1 ; C=CC(=O)N1CCN(C2CCCC/C=C/CCN3C(=O)OCc4cnc(nc43)N[C@@H](C)c3ccc2cc3)CC1] 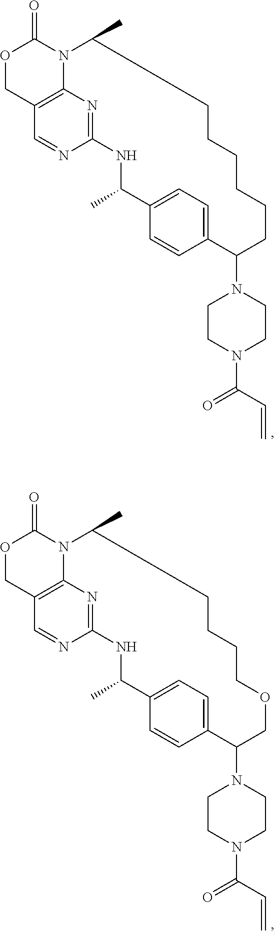 [SMILES: C=CC(=O)N1CCN(C2CCCCCC[C@H](C)N3C(=O)OCc4cnc(nc43)N[C@@H](C)c3ccc2cc3)CC1 ; C=CC(=O)N1CCN(C2COCCCC[C@H](C)N3C(=O)OCc4cnc(nc43)N[C@@H](C)c3ccc2cc3)CC1] 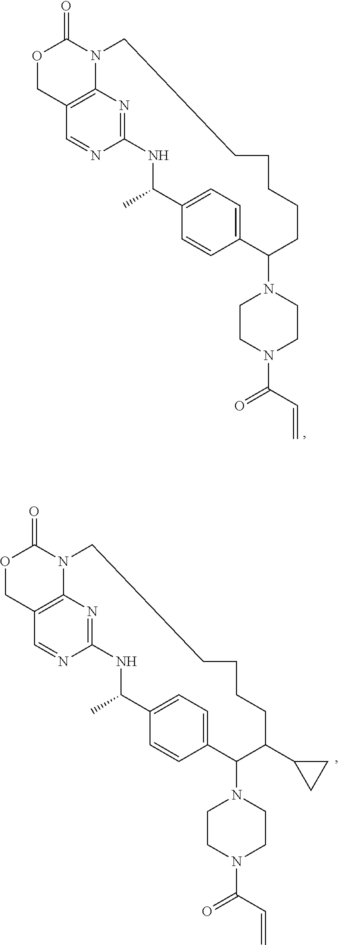 [SMILES: C=CC(=O)N1CCN(C2CCCCCCN3C(=O)OCc4cnc(nc43)N[C@@H](C)c3ccc2cc3)CC1 ; C=CC(=O)N1CCN(C2c3ccc(cc3)[C@H](C)Nc3ncc4c(n3)N(CCCCCC2C2CC2)C(=O)OC4)CC1] 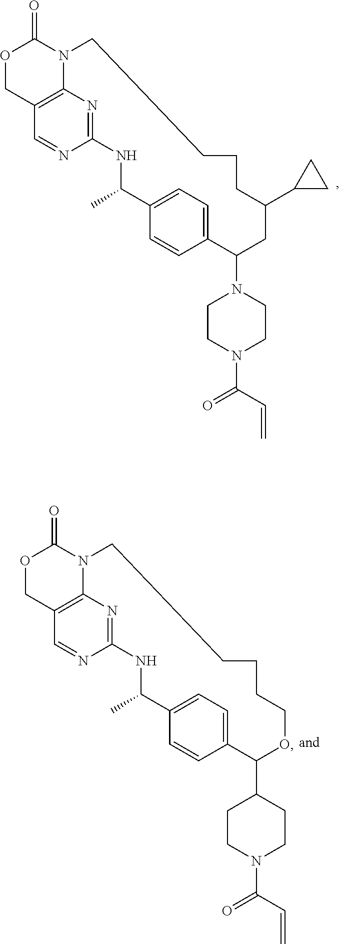 [SMILES: C=CC(=O)N1CCN(C2CC(C3CC3)CCCCN3C(=O)OCc4cnc(nc43)N[C@@H](C)c3ccc2cc3)CC1 ; C=CC(=O)N1CCC(C2OCCCCCN3C(=O)OCc4cnc(nc43)N[C@@H](C)c3ccc2cc3)CC1] 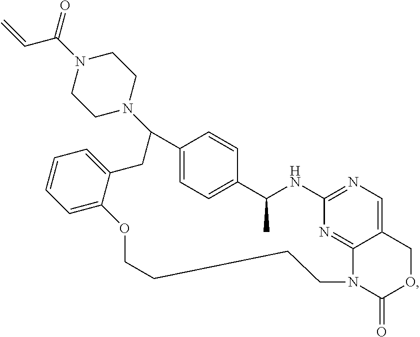 [SMILES: C=CC(=O)N1CCN(C2Cc3ccccc3OCCCCN3C(=O)OCc4cnc(nc43)N[C@@H](C)c3ccc2cc3)CC1]
or a pharmaceutically acceptable salt thereof.

32. A pharmaceutical formulation comprising a compound as recited in claim 1, or a pharmaceutically acceptable salt thereof, together with a pharmaceutically acceptable carrier.

33. (canceled)

34. (canceled)

35. A method of treatment of a disease associated with an IDH protein having one or more neomorphic mutations, comprising the administration of a therapeutically effective amount of a compound as recited in claim 1, or a pharmaceutically acceptable salt thereof.

36. (canceled)

37. (canceled)

38. (canceled)

39. (canceled)

40. (canceled)

41. (canceled)

42. The method as recited in claim 35, wherein the disease is cancer.

43. The method as recited in claim 42, wherein the cancer is chosen from Melanoma, Malignant Solid Tumors, Colorectal Carcinoma, Non-Small Cell Lung Carcinoma, Acute Myeloid Leukemia, Myelodysplastic Syndromes, Chronic Myelomonocytic Leukemia, Colorectal Adenocarcinoma, Multiple Myeloma, Non-Hodgkin Lymphoma, Pancreatic Carcinoma, Cutaneous Melanoma, Ovarian Carcinoma, Pancreatic Ductal Adenocarcinoma, Acute Lymphoblastic Leukemia, Thyroid Gland Carcinoma, Glioma, Neurofibromatosis, Poorly Differentiated Thyroid Gland Carcinoma, Myelodysplastic Syndrome With Excess Blasts, Juvenile Myelomonocytic Leukemia, Histiocytic And Dendritic Cell Neoplasm, Head And Neck Squamous Cell Carcinoma, Small Cell Lung Carcinoma, Low Grade Glioma, Squamous Cell Lung Carcinoma, Breast Carcinoma, Chronic Myelomonocytic Leukemia, Thyroid Gland Undifferentiated (Anaplastic) Carcinoma, Embryonal Rhabdomyosarcoma, Thyroid Gland Follicular Carcinoma, T-Cell Acute Lymphoblastic Leukemia, Mucosal Melanoma, Low Grade Ovarian Serous Adenocarcinoma, Thyroid Gland Papillary Carcinoma, Refractory Anemia With Excess Blasts, Myeloid Neoplasm, Myelodysplastic/Myeloproliferative Neoplasm, Rectal Carcinoma, Colon Carcinoma, Malignant Peripheral Nerve Sheath Tumor, Cholangiocarcinoma, Endometrial Carcinoma, Mantle Cell Lymphoma, Secondary Myelodysplastic Syndrome, Therapy-Related Myelodysplastic Syndrome, Lymphoma, Neuronal And Mixed Neuronal-Glial Tumors, Ganglioglioma, Soft Tissue Sarcoma, Bladder Carcinoma, Esophageal Carcinoma, Sarcoma, Thymic Carcinoma, Lung Adenocarcinoma, Lung Carcinoma, Uveal Melanoma, Head And Neck Carcinoma, Diffuse Glioma, Squamous Cell Carcinoma, Chronic Myeloid Leukemia, Adenocarcinoma of the Gastroesophageal Junction, Glioblastoma, Neuroblastoma, Astrocytic Tumor, Hepatocellular Carcinoma, Pancreatic Adenocarcinoma, Diffuse Large B-Cell Lymphoma, Anaplastic Astrocytoma, Gastric Adenocarcinoma, Gastric Carcinoma, Prostate Carcinoma, Renal Cell Carcinoma, B-Cell Acute Lymphoblastic Leukemia, Double-Hit Lymphoma, Dysembryoplastic Neuroepithelial Tumor, Gangliocytoma, Low-Grade Neuroepithelial Tumor, Peripheral T-Cell Lymphoma, Pilocytic Astrocytoma, Pilomyxoid Astrocytoma, Rhabdoid Tumor, and Schwannoma.

44. The method as recited in claim 43, wherein the cancer is Acute Myeloid Leukemia.

45. The method as recited in claim 43, wherein the cancer is chosen from Glioma, Diffuse Glioma, Ganglioglioma, and Low Grade Glioma.
